(19) United States
(12) Patent Application Publication
(10) Pub. No.: US 2012/0238488 A1
(43) Pub. Date: Sep. 20, 2012

(54) COMPOUNDS AND METHODS FOR TREATING NEOPLASIA

(76) Inventors: Konstantinos Alevizopoulos, Giez (CH); Theodora Calogeropoulou, Athens (GR); Christos Stournaras, Heraklion (GR)

(21) Appl. No.: 13/394,987

(22) PCT Filed: Jun. 22, 2010
(86) PCT No.: PCT/EP10/58842

§ 371 Date: May 29, 2012

Related U.S. Application Data
(60) Provisional application No. 61/240,540, filed on Sep. 8, 2009.

(51) Int. Cl. A61K 31/58; C12N 5/09; A61K 31/568; A61K 31/5685; A61P 31/04; A61K 31/704; A61K 38/02; A61K 31/7048; A61P 35/00; C07J 41/00; C07J 43/00

(52) U.S. Cl. 514/1.1; 552/520; 435/375; 514/182; 514/177; 540/106; 514/176; 514/171; 514/34; 514/27

(57) ABSTRACT

The invention features compounds, pharmaceutical compositions and methods useful for the treatment of neoplasia. In particular embodiments, the compounds of the invention are useful for the treatment of multidrug resistant neoplasia.

### RELATED APPLICATION

This application claims priority to U.S. provisional application Ser. No. 61/240,540, filed Sep. 8, 2009, and the subject matter described herein is related to the subject matter described in U.S. provisional application Ser. No. 61/219,327, filed Jun. 22, 2009. The entire disclosures of these applications are incorporated herein by this reference.

### BACKGROUND OF THE INVENTION

Approximately one million Americans are diagnosed with neoplasia every year, and about half a million people in the United States die of the disease annually. Although improvements in neoplasia detection, diagnosis, and treatment have increased the survival rate for many types of neoplasia, only about 60 percent of people diagnosed with neoplasia are alive five years after treatment, making neoplasia the second leading cause of death in the United States. One of the reasons for this poor long term survival rate is that many patients develop multidrug resistant neoplasias. After several cycles of chemotherapy, some tumor cells become resistant not only to the agent used in the chemotherapy, but also to compounds with different structures and mechanisms of action. It is believed that the ATP binding cassette superfamily of transporter proteins acts as an energy-dependent drug efflux pump and alterations in these transporter proteins are associated with the development of multi-drug resistant neoplasias. The activity of this family of proteins prevents the intracellular accumulation of a broad range of cytotoxic drugs.

### SUMMARY OF THE INVENTION

One aspect of the invention provides a compound of Formula (I)

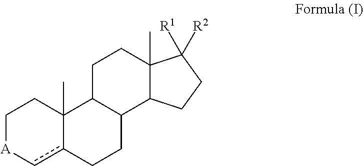

wherein:

[symbol] represents a single bond or double bond in the ring structure of Formula (I);

One of R¹ and R² is H, and the other is —OR³; alkyl optionally substituted with hydroxyl or alkoxy; —C(O)—NH₂; —C(O)—O-alkyl; —NHR⁸; alkynyl; or R¹ and R² together with the carbon to which they are attached form C═O, C═C(R⁹)₂, or C═N—OR¹⁰;

R³ is H; —NO₂; carbonylalkyl; carbonylaryl; or aminoalkyl optionally substituted with one or more alkyl;

A is CH(OR⁴), C═N—OR⁵, CH—CH═N—OR⁶, C═CH—CH═N—OR⁶, CH—CH═CH—R⁶, CH—SR⁷, or CH—S(O)R⁷;

R⁴ is alkyl optionally substituted by a 4- to 6-membered heterocyclic ring, a 5 to 14-membered heteroaryl, amino(C₁₋C₄)alkoxyl, (C₁₋C₄)alkoxyl substituted by a 4- or 6-membered heterocyclic ring, or guanidinyl; aminoalkyl optionally substituted by one or more alkyl; or aminoacyl optionally substituted by alkyl; wherein said heterocyclic or heteroaryl ring is further optionally substituted by one or more alkyl

R⁵ is aminoalkyl optionally substituted by one or more alkyl or acetyl; alkyl optionally substituted by guanidinyl, heteroaryl that is further optionally substituted by one or more alkyl or aminoalkyl optionally substituted by one or more alkyl, or a 4- to 6-membered heterocyclic ring that is further optionally substituted by one or more alkyl; or a 4- to 6-membered heterocyclic ring optionally substituted by one or more alkyl;

R⁶ and R⁷, each independently, are aminoalkyl optionally substituted by one or more alkyl;

R⁸ is H or formyl;

R⁹, for each occurrence, is the same or different and is H or halogen;

R¹⁰ is H or alkyl; and

tautomers, stereoisomers, Z and E isomers, optical isomers, N-oxides, hydrates, polymorphs, pharmaceutically acceptable esters, salts, prodrugs and/or isotopic derivatives thereof.

One embodiment provides that the [symbol] in Formula (I) represents a single bond. Another embodiment provides that the [symbol] represents a double bond.

A separate embodiment provides a compound of Formula (I) wherein R¹ and R² together with the carbon atom to which they are attached form C═O. One embodiment provides that A is C═N—OR⁵. In an embodiment, R⁵ is amino(C₁-C₄)alkyl that is optionally substituted by one or more (C₁-C₄)alkyl or acetyl groups.

Examples of R⁵ include, but are not limited to, aminoethyl, 2-aminopropyl, 2-methyl-2-aminopropyl, 2-aminomethyl-2-propyl, N-methylamino-ethyl, aminopropyl, N-methylamino-propyl, aminobutyl, N-ethylamino-butyl, N,N-dimethylamino-ethyl, and N-acetylamino-ethyl.

Certain compounds of Formula (I) include compounds as follows:

(10S,13S)-3-(2-aminoethoxyimino)-10,13-dimethyltetradecahydro-1H-cyclopenta[a]phenanthren-17(2H)-one (Compound 1):

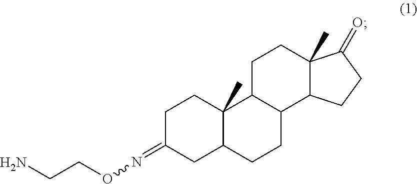

(10S,13 S)-3-(2-aminopropoxyimino)-10,13-dimethyltetradecahydro-1H-cyclopenta[a]phenanthren-17(2H)-one (Compound 2):

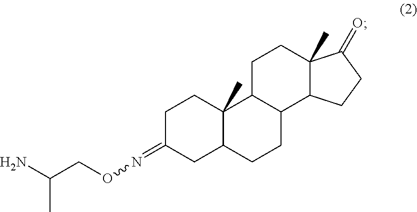

(10S,13 S)-3-(2-amino-2-methylpropoxyimino)-10,13-dimethyltetradecahydro-1H-cyclopenta[a]phenanthren-17(2H)-one (Compound 3):

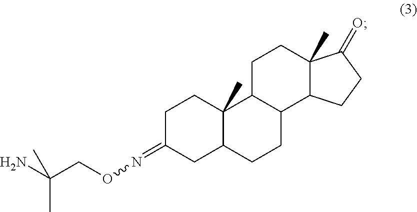

(10S,13S)-3-(1-amino-2-methylpropan-2-yloxyimino)-10,13-dimethyltetradecahydro-1H-cyclopenta[a]phenanthren-17(2H)-one (Compound 4):

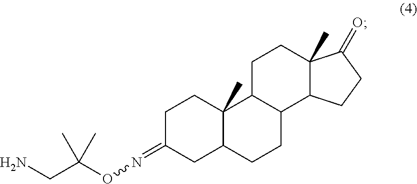

(10S,13S)-10,13-dimethyl-3-(2-(methylamino)ethoxyimino)tetradecahydro-1H-cyclopenta[a]phenanthren-17(2H)-one (Compound 5):

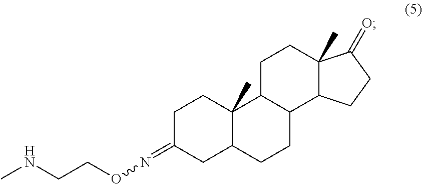

(10S,13S)-3-(3-aminopropoxyimino)-10,13-dimethyltetradecahydro-1H-cyclopenta[a]phenanthren-17(2H)-one (Compound 6):

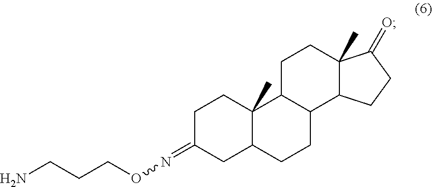

(10S,13S)-10,13-dimethyl-3-(3-(methylamino)propoxyimino)tetradecahydro-1H-cyclopenta[a]phenanthren-17(2H)-one (Compound 7):

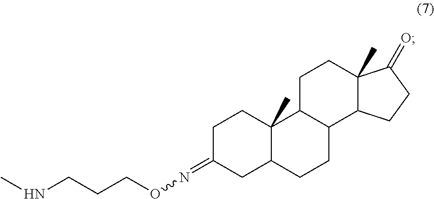

(10S,13S)-3-(4-aminobutoxyimino)-10,13-dimethyltetradecahydro-1H-cyclopenta[a]phenanthren-17(2H)-one (Compound 8):

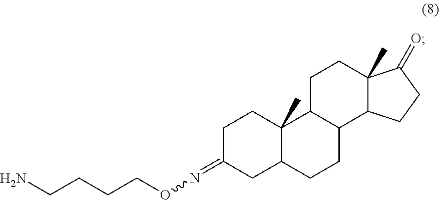

(10S,13S)-3-(4-(ethylamino)butoxyimino)-10,13-dimethyltetradecahydro-1H-cyclopenta[a]phenanthren-17(2H)-one (Compound 9):

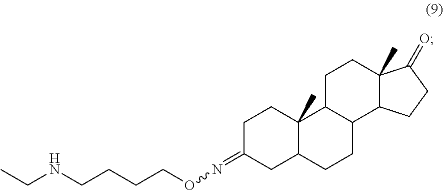

(10S,13S)-3-(2-(dimethylamino)ethoxyimino)-10,13-dimethyltetradecahydro-1H-cyclopenta[a]phenanthren-17(2H)-one (Compound 10):

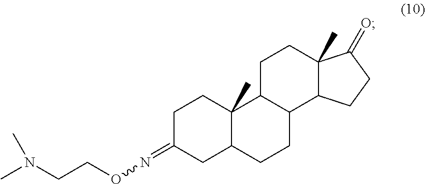

N-(2-((10S,13S)-10,13-dimethyl-17-oxohexahydro-1H-cyclopenta[a]phenanthren-3 (2H,4H,10H,12H,13H,14H,15H,16H,17H)-ylideneaminooxy)ethyl)acetamide (Compound 11):

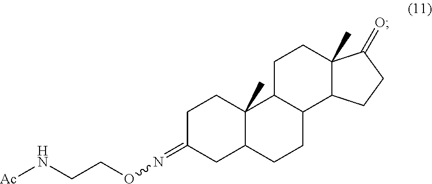

(10R,13S)-3-(2-amino ethoxyimino)-10,13-dimethyl-2,3,7,8,9,10,11,12,13,14,15,16-dodecahydro-1H-cyclopenta[a]phenanthren-17(6H)-one (Compound 12):

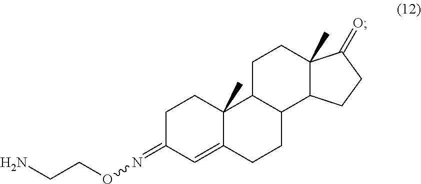

(10R,135)-3-(2-aminopropoxyimino)-10,13-dimethyl-2,3,7,8,9,10,11,12,13,14,15,16-dodecahydro-1H-cyclopenta[a]phenanthren-17(6H)-one (Compound 13):

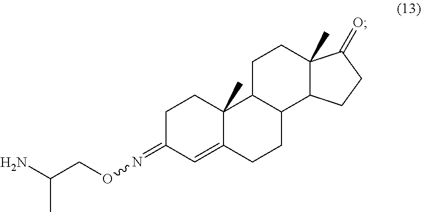

(10R,13S)-3-(2-amino-2-methylpropoxyimino)-10,13-dimethyl-2,3,7,8,9,10, 11,12,13,14,15,16-dodecahydro-1H-cyclopenta[a]phenanthren-17(6H)-one (Compound 14):

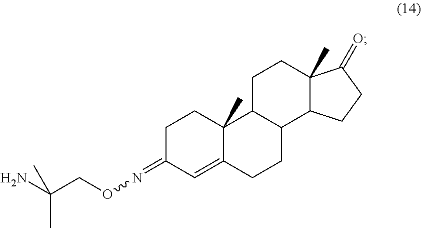

(10R,13S)-3-(1-amino-2-methylpropan-2-yloxyimino)-10,13-dimethyl-2,3,7,8,9,10,11,12,13,14,15,16-dodecahydro-1H-cyclopenta[a]phenanthren-17(6H)-one (Compound 15):

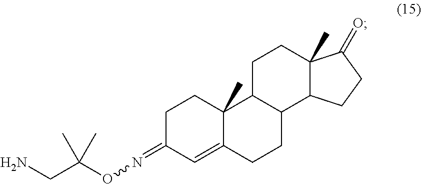

(10R,13S)-10,13-dimethyl-3-(2-(methylamino)ethoxyimino)-2,3,7,8,9,10,11,12,13,14,15,16-dodecahydro-1H-cyclopenta[a]phenanthren-17(6H)-one (Compound 16):

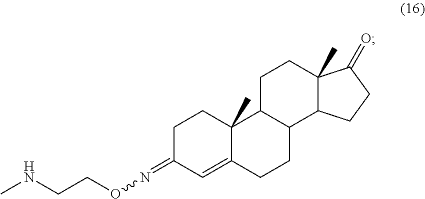

(10R,13S)-3-(3-aminopropoxyimino)-10,13-dimethyl-2,3,7,8,9,10,11,12,13,14,15,16-dodecahydro-1H-cyclopenta[a]phenanthren-17(6H)-one (Compound 17):

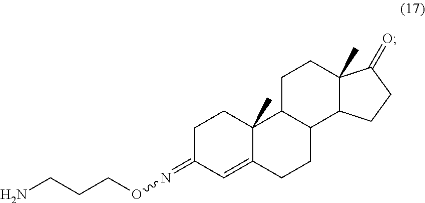

(10R,13S)-10,13-dimethyl-3-(3-(methylamino)propoxyimino)-2,3,7,8,9,10,11,12,13,14,15,16-dodecahydro-1H-cyclopenta[a]phenanthren-17(6H)-one (Compound 18):

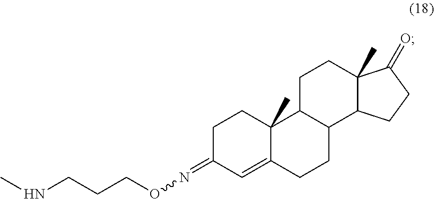

(10R,13S)-3-(4-aminobutoxyimino)-10,13-dimethyl-2,3,7,8,9,10,11,12,13,14,15,16-dodecahydro-1H-cyclopenta[a]phenanthren-17(6H)-one (Compound 19):

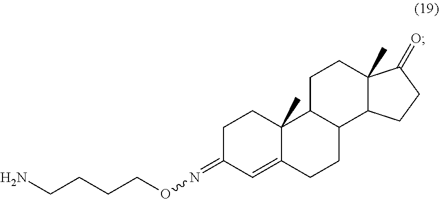

(10R,13S)-3-(4-(ethylamino)butoxyimino)-10,13-dimethyl-2,3,7,8,9,10,11,12,13,14,15,16-dodecahydro-1H-cyclopenta[a]phenanthren-17(6H)-one (Compound 20):

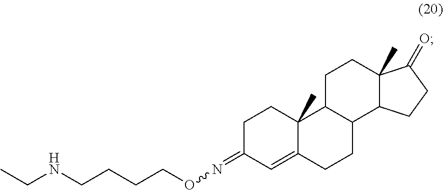

(10R,13S)-3-(2-(dimethylamino)ethoxyimino)-10,13-dimethyl-2,3,7,8,9,10,11,12,13,14,15,16-dodecahydro-1H-cyclopenta[a]phenanthren-17(6H)-one (Compound 21):

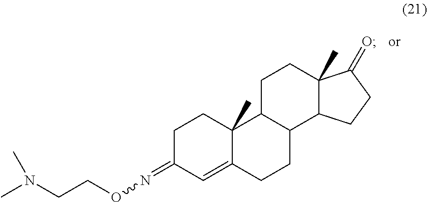

N-(2-((10R,13S)-10,13-dimethyl-17-oxo-7,8,9,11-tetrahydro-1H-cyclopenta [a]phenanthren-3 (2H,6H,10H,12H,13H,14H,15H,16H,17H)-ylidene amino oxy)ethyl)acetamide (Compound 22):

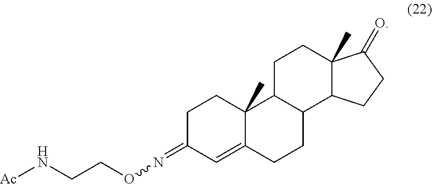

Another embodiment provides a compound of Formula (I) wherein A is C═N—OR⁵, and R⁵ is (C₁-C₄)alkyl that is optionally substituted by guanidinyl, a 5- to 14-membered heteroaryl or a 4- to 6-membered heterocyclic ring, wherein 5- to 14-membered heteroaryl is optionally substituted by one or more (C₁-C₄)alkyl or amino(C₁-C₄)alkyl that is further optionally substituted by one or more (C₁-C₄)alkyl, and said 4- to 6-membered heterocyclic ring is optionally substituted by one or more (C₁-C₄)alkyl.

One instance is that R⁵ is (C₁-C₄)alkyl substituted by guanidinyl. Exemplified compounds of Formula (I) include:

1-(2-((10S,13S)-10,13-dimethyl-17-oxohexahydro-1H-cyclopenta[a]phenanthren-3(2H,4H,10H,12H,13H, 14H,15H,16H,17H)-ylideneaminooxy)ethyl)guanidine (Compound 23):

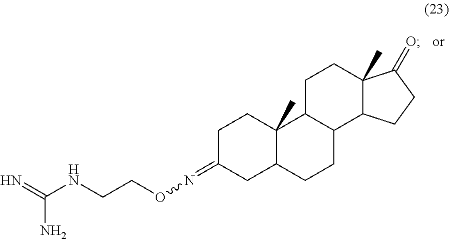

1-(2-((10R,13S)-10,13-dimethyl-17-oxo-7,8,9,11-tetrahydro-1H-cyclopenta[a]phenanthren-3 (2H,6H,10H,12H,13H,14H,15H,16H,17H)-ylideneaminooxy)ethyl)guanidine (Compound 24):

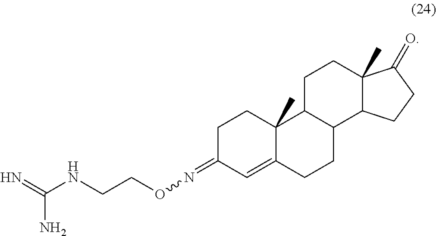

Another instance is that R⁵ is (C₁-C₄)alkyl substituted by a 5- to 14-membered heteroaryl that is optionally substituted by one or more (C₁-C₄)alkyl or amino(C₁-C₄)alkyl, wherein said amino(C₁-C₄)alkyl is further optionally substituted by one or more (C₁-C₄)alkyl.

In one embodiment, R⁵ is (C₁-C₄)alkyl substituted by a 5- or 6-membered heteroaryl that is optionally substituted by one or more (C₁-C₄)alkyl. Said 5- or 6-membered heteroaryl group is, for example, an oxazolyl, isoxazolyl or oxadiazolyl ring, which is further optionally substituted by one or more (C₁-C₄)alkyl.

Examples of compounds of Formula (I) include:

(10S,13S)-10,13-dimethyl-3-(2-(5-methylisoxazol-3-yl)ethoxyimino)tetradecahydro-1H-cyclopenta[a]phenanthren-17(2H)-one (Compound 25):

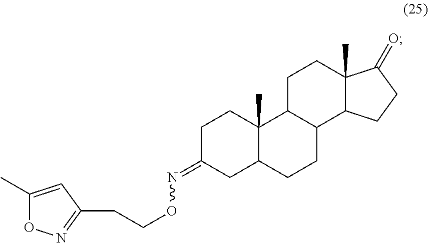

(10S,13S)-10,13-dimethyl-3-(2-(3-methyl-1,2,4-oxadiazol-5-yl)ethoxy-imino)tetradecahydro-1H-cyclopenta[a]phenanthren-17(2H)-one (Compound 26):

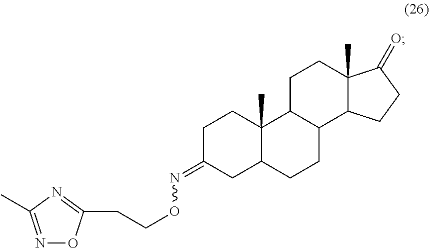

(10S,13S)-10,13-dimethyl-3-(2-(2-methyloxazol-5-yl)ethoxyimino)tetradecahydro-1H-cyclopenta[a]phenanthren-17(2H)-one (Compound 27):

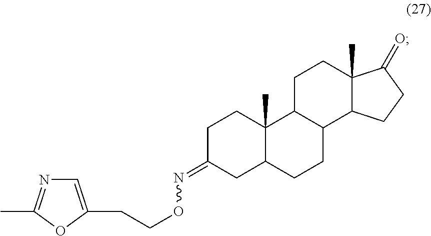

(10R,13S)-10,13-dimethyl-3-(2-(5-methylisoxazol-3-yl)ethoxyimino)-2,3,7,8,9,10,11,12,13,14,15,16-dodecahydro-1H-cyclopenta[a]phenanthren-17(6H)-one (Compound 28):

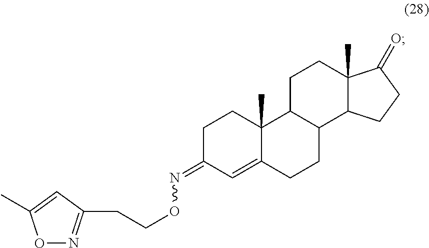

(10R,13S)-10,13-dimethyl-3-(2-(3-methyl-1,2,4-oxadiazol-5-yl)ethoxyimino)-2,3,7,8,9,10,11,12,13,14,15,16-dodecahydro-1H-cyclopenta[a]phenanthren-17(6H)-one (Compound 29):

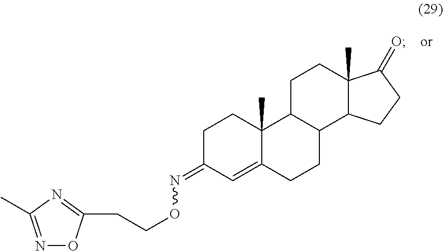

(10R,13S)-10,13-dimethyl-3-(2-(2-methyloxazol-5-yl)ethoxyimino)-2,3,7,8,9,10,11,12,13,14,15,16-dodecahydro-1H-cyclopenta[a]phenanthren-17(6H)-one (Compound 30):

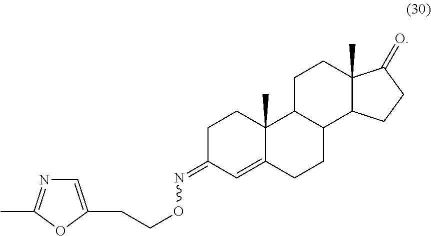

In another embodiment, R⁵ is (C₁-C₄)alkyl substituted by a 5- or 6-membered heteroaryl that is substituted by amino(C₁-C₄)alkyl, and said amino(C₁-C₄)alkyl is optionally substituted by one or more (C₁-C₄)alkyl. One instance provides that said 5- or 6-membered heteroaryl is a triazolyl ring substituted by amino(C₁-C₄)alkyl that is further optionally substituted by one or more (C₁-C₄)alkyl. Another instance provides that said 5- or 6-membered heteroaryl is an isoxazolyl or oxazolyl ring, wherein said isoxazolyl or oxazolyl ring is substituted by amino(C₁-C₄)alkyl that is further optionally substituted by one or more (C₁-C₄)alkyl. Another instance of the said 5- or 6-membered heteroaryl is an oxadiazolyl ring substituted by amino(C₁-C₄)alkyl, which is further optionally substituted by one or more (C₁-C₄)alkyl.

Exemplified compounds of Formula (I) include, but are not limited to,

(10S,13S)-3-(2-(4-(aminomethyl)-1H-1,2,3-triazol-1-yl)ethoxyimino)-10,13-dimethyltetradecahydro-1H-cyclopenta[a]phenanthren-17(2H)-one (Compound 31):

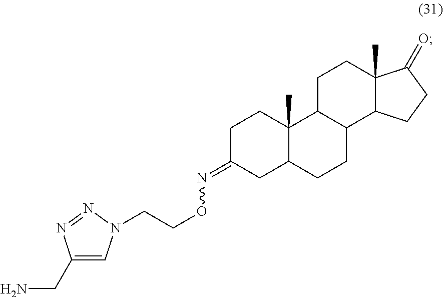

(10S,13S)-3-(2-(4-(2-amino ethyl)-1H-1,2,3-triazol-1-yl)ethoxyimino)-10,13-dimethyltetradecahydro-1H-cyclopenta[a]phenanthren-17(2H)-one (Compound 32):

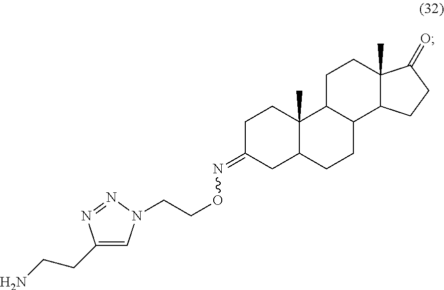

(10S,13S)-3-(2-(4-((dimethylamino)methyl)-1H-1,2,3-triazol-1-yl)ethoxyimino)-10,13-dimethyltetradecahydro-1H-cyclopenta[a]phenanthren-17(2H)-one (Compound 33):

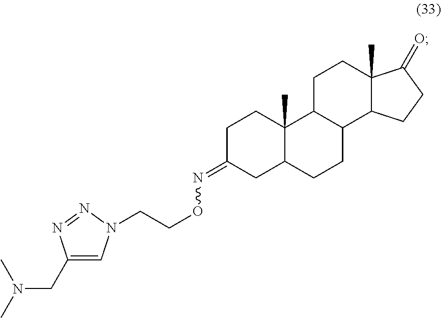

(10S,13S)-3-(2-(4-(2-(dimethylamino)ethyl)-1H-1,2,3-triazol-1-yl)ethoxyimino)-10,13-dimethyltetradecahydro-1H-cyclopenta[a]phenanthren-17(2H)-one (Compound 34):

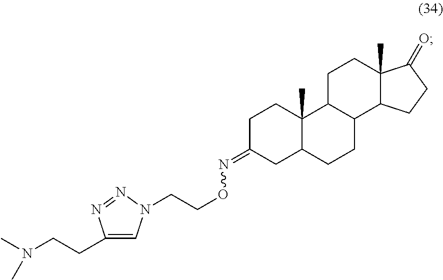

(10S,13S)-3-(2-(4-(aminomethyl)-1H-1,2,3-triazol-1-yl)butoxyimino)-10,13-dimethyltetradecahydro-1H-cyclopenta[a]phenanthren-17(2H)-one (Compound 196):

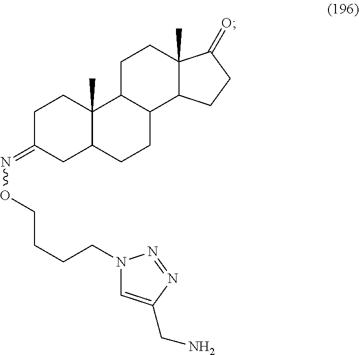

(10R,13S)-3-(2-(4-(aminomethyl)-1H-1,2,3-triazol-1-yl)ethoxyimino)-10,13-dimethyl-2,3,7,8,9,10,11,12,13,14,15,16-dodecahydro-1H-cyclopenta[a]phenanthren-17(6H)-one (Compound 35)

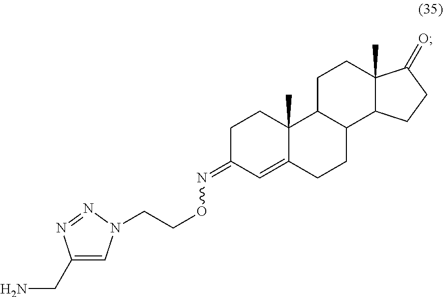

(10R,13S)-3-(2-(4-((dimethylamino)methyl)-1H-1,2,3-triazol-1-yl)ethoxyimino)-10,13-dimethyl-2,3,7,8,9,10,11,12,13,14,15,16-dodecahydro-1H-cyclopenta[a]phenanthren-17(6H)-one (Compound 36):

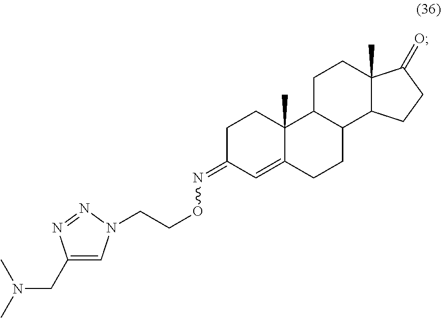

(10R,13S)-3-(2-(4-(2-aminoethyl)-1H-1,2,3-triazol-1-yl)ethoxyimino)-10,13-dimethyl-2,3,7,8,9,10,11,12,13,14,15,16-dodecahydro-1H-cyclopenta[a]phenanthren-17(6H)-one (Compound 37):

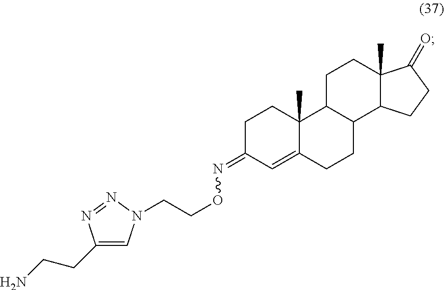

(10R,13S)-3-(2-(4-(2-(dimethylamino)ethyl)-1H-1,2,3-triazol-1-yl)ethoxyimino)-10,13-dimethyl-2,3,7,8,9,10,11,12,13,14,15,16-dodecahydro-1H-cyclopenta[a]phenanthren-17(6H)-one (Compound 38):

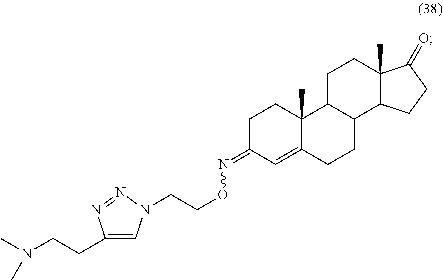

(10S,13S)-3-(2-(4-(aminomethyl)-1H-1,2,3-triazol-1-yl)butoxyimino)-10,13-dimethyl 2,3,7,8,9,10,11,12,13,14,15,16-dodecahydro-1H-cyclopenta[a]phenanthren-17(6H)-one (Compound 197):

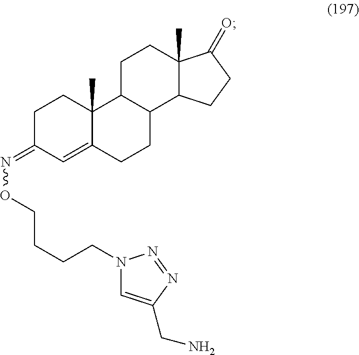

(10S,13S)-3-(2-(5-(aminomethyl) is oxazol-3-yl)ethoxyimino)-10,13-dimethyltetradeca-hydro-1H-cyclopenta[a]phenanthren-17(2H)-one (Compound 39):

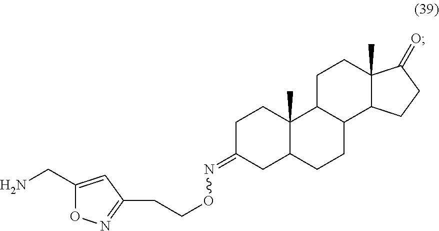

(10S,13S)-3-(2-(2-(aminomethyl)oxazol-5-yl)ethoxyimino)-10,13-dimethyltetradeca-hydro-1H-cyclopenta[a]phenanthren-17(2H)-one (Compound 40):

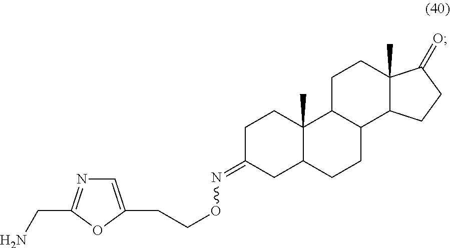

(10S,13S)-3-(2-(5-(aminomethyl)oxazol-2-yl)ethoxyimino)-10,13-dimethyltetradeca-hydro-1H-cyclopenta[a]phenanthren-17(2H)-one (Compound 41):

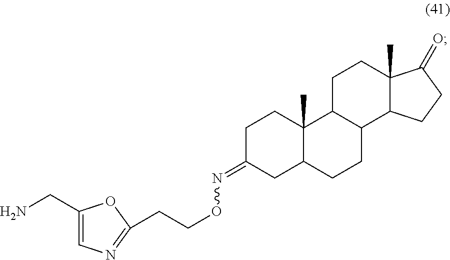

(10S,13S)-3-(2-(2-((dimethylamino)methyl)oxazol-5-yl)ethoxyimino)-10,13-dimethyltetradecahydro-1H-cyclopenta[a]phenanthren-17(2H)-one (Compound 42):

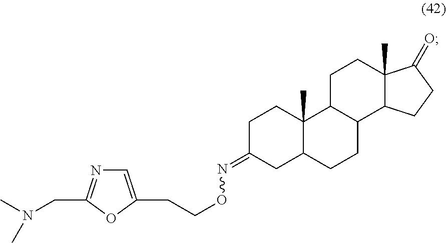

(10S,13S)-3-(2-(5-((dimethylamino)methyl)oxazol-2-yl)ethoxyimino)-10,13-dimethyltetradecahydro-1H-cyclopenta[a]phenanthren-17(2H)-one (Compound 43):

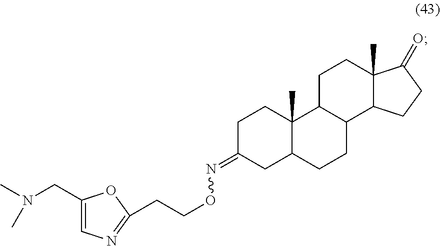

(10R,13S)-3-(2-(5-(aminomethyl) is oxazol-3-yl)ethoxyimino)-10,13-dimethyl-2,3,7,8,9,10,11,12,13,14,15,16-dodecahydro-1H-cyclopenta[a]phenanthren-17(6H)-one (Compound 44):

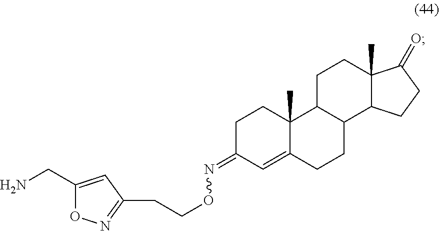

(10R,13S)-3-(2-(2-(aminomethyl)oxazol-5-yl)ethoxyimino)-10,13-dimethyl-2,3,7,8,9,10,11,12,13,14,15,16-dodecahydro-1H-cyclopenta[a]phenanthren-17(6H)-one (Compound 45):

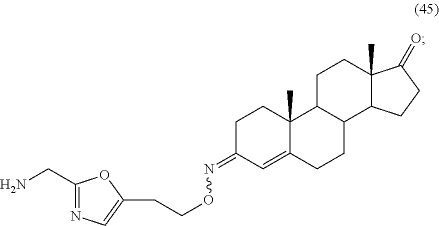

(10R,13S)-3-(2-(2-((dimethylamino)methyl)oxazol-5-yl)ethoxyimino)-10,13-dimethyl-2,3,7,8,9,10,11,12,13,14,15,16-dodecahydro-1H-cyclopenta[a]phenanthren-17(6H)-one (Compound 46):

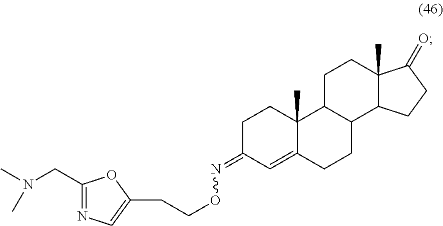

(10R,13S)-3-(2-(5-(aminomethyl)oxazol-2-yl)ethoxyimino)-10,13-dimethyl-2,3,7,8,9,10,11,12,13,14,15,16-dodecahydro-1H-cyclopenta[a]phenanthren-17(6H)-one (Compound 47):

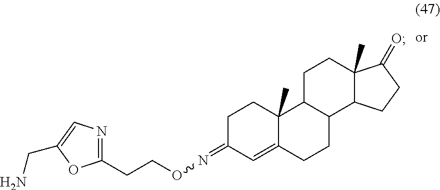

(10R,13S)-3-(2-(5-((dimethylamino)methyl)oxazol-2-yl)ethoxyimino)-10,13-dimethyl-2,3,7,8,9,10,11,12,13,14,15,16-dodecahydro-1H-cyclopenta[a]phenanthren-17(6H)-one (Compound 48):

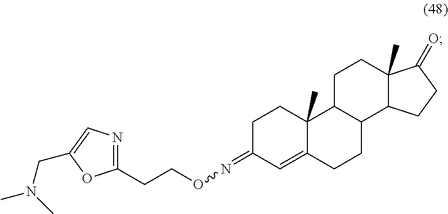

(10S,13S)-3-(2-(3-(aminomethyl)-1,2,4-oxadiazol-5-yl)ethoxyimino)-10,13-dimethyltetradecahydro-1H-cyclopenta[a]phenanthren-17(2H)-one (Compound 49):

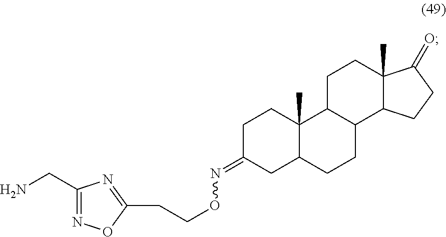

(10S,13S)-3-(2-(3-((dimethylamino)methyl)-1,2,4-oxadiazol-5-yl)ethoxyimino)-10,13-dimethyltetradecahydro-1H-cyclopenta[a]phenanthren-17(2H)-one (Compound 50):

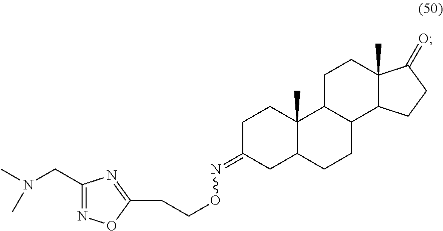

(10R,135)-3-(2-(3-(aminomethyl)-1,2,4-oxadiazol-5-yl)ethoxyimino)-10,13-dimethyl-2,3,7,8,9,10,11,12,13,14,15,16-dodecahydro-1H-cyclopenta[a]phenanthren-17(6H)-one (Compound 51):

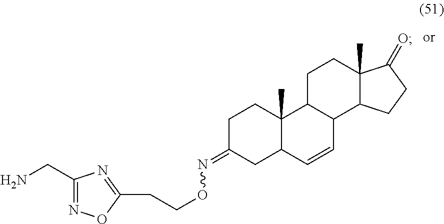

(10R,13S)-3-(2-(3-((dimethylamino)methyl)-1,2,4-oxadiazol-5-yl)ethoxyimino)-10,13-dimethyl-2,3,7,8,9,10,11,12,13,14,15,16-dodecahydro-1H-cyclopenta[a]phenanthren-17(6H)-one (Compound 52):

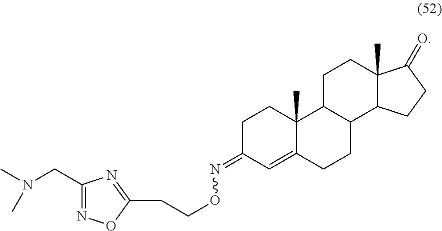

Another embodiment provides that a compound of Formula (I), wherein A is C═N—OR⁵, R⁵ is (C₁-C₄)alkyl substituted by a 4- to 6-membered heterocyclic ring, which is further optionally substituted by one or more (C₁-C₄)alkyl. One instance provides that R⁵ is (C₁-C₄)alkyl substituted by a pyrrolidinyl ring.

Compounds of Formula (I) include, for example, (10S,13S)-10,13-dimethyl-3-(pyrrolidin-2-ylmethoxyimino)tetradecahydro-1H-cyclopenta[a]phenanthren-17(2H)-one (Compound 53):

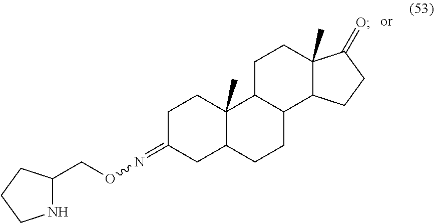

(10R,13S)-10,13-dimethyl-3-(pyrrolidin-2-ylmethoxyimino)-2,3,7,8,9,10,11,12,13,14,15,16-dodecahydro-1H-cyclopenta[a]phenanthren-17(6H)-one (Compound 54):

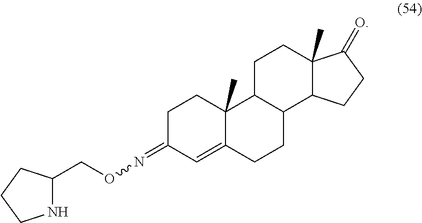

In one embodiment, a compound of Formula (I) has A being C═N—OR⁵, wherein R⁵ is a 4- to 6-membered heterocyclic ring that is optionally substituted by one or more (C₁-C₄)alkyl. Said R⁵ group can be, but is not limited to, a pyrrolidinyl, azetidinyl, or piperidinyl ring, which is optionally substituted by one or more (C₁-C₄)alkyl. Compounds of Formula (I) include, but are not limited to,

(10S,13S)-3-(azetidin-3-yloxyimino)-10,13-dimethyltetradec ahydro-1H-cyclopenta[a]phenanthren-17(2H)-one (Compound 55):

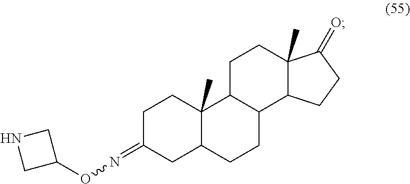

(10S,13S)-10,13-dimethyl-3-(pyrrolidin-3-yloxyimino)tetradecahydro-1H-cyclopenta[a]phenanthren-17(2H)-one (Compound 56):

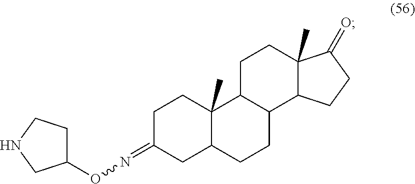

(10S,13S)-10,13-dimethyl-3-((S)-1-methylpyrrolidin-3-yloxyimino)tetradecahydro-1H-cyclopenta[a]phenanthren-17(2H)-one (Compound 57):

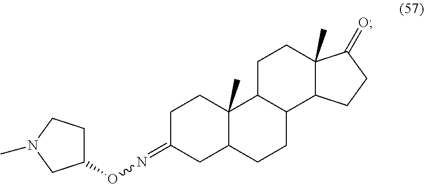

(10S,13S)-10,13-dimethyl-3-(piperidin-3-yloxyimino)tetradecahydro-1H-cyclopenta[a]phenanthren-17(2H)-one (Compound 58):

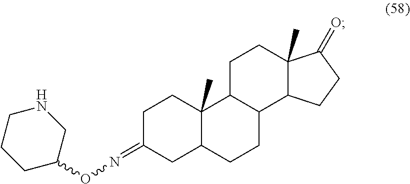

(10S,13S)-10,13-dimethyl-3-(piperidin-4-yloxyimino)tetradecahydro-1H-cyclopenta[a]phenanthren-17(2H)-one (Compound 59):

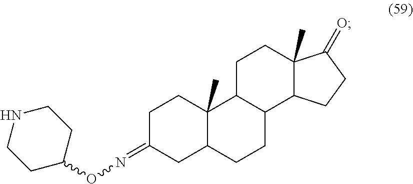

(10R,13S)-3-(azetidin-3-yloxyimino)-10,13-dimethyl-2,3,7,8,9,10,11,12,13,14,15,16-dodecahydro-1H-cyclopenta[a]phenanthren-17(6H)-one (Compound 60):

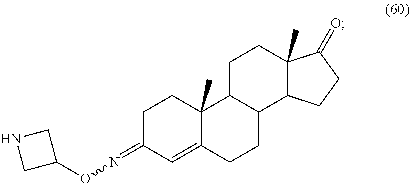

(10R,13S)-10,13-dimethyl-3-(pyrrolidin-3-yloxyimino)-2,3,7,8,9,10,11,12,13,14,15,16-dodecahydro-1H-cyclopenta[a]phenanthren-17(6H)-one (Compound 61):

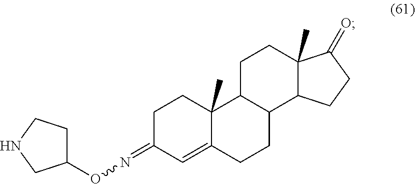

(10R,13S)-10,13-dimethyl-3-((S)-1-methylpyrrolidin-3-yloxyimino)-2,3,7,8,9,10,11,12,13,14,15,16-dodecahydro-1H-cyclopenta[a]phenanthren-17(6H)-one (Compound 62):

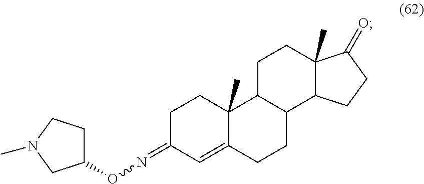

(10R,13S)-10,13-dimethyl-3-(piperidin-3-yloxyimino)-2,3,7,8,9,10,11,12,13,14,15,16-dodecahydro-1H-cyclopenta[a]phenanthren-17(6H)-one (Compound 63):

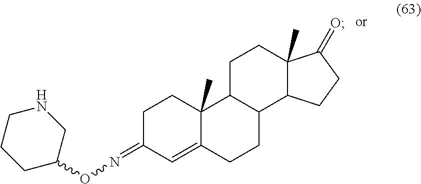

(10R,13S)-10,13-dimethyl-3-(piperidin-4-yloxyimino)-2,3,7,8,9,10,11,12,13,14,15,16-dodecahydro-1H-cyclopenta[a]phenanthren-17(6H)-one (Compound 64):

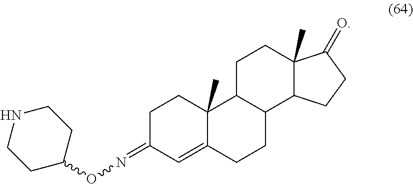

In one embodiment, A in Formula (I) represents CH(OR⁴). One embodiment provides that R⁴ is (C₁-C₄)alkyl that is optionally substituted by a 4- to 6-membered heterocyclic ring, a 5 to 14-membered heteroaryl, amino(C₁₋C₄)alkoxyl, (C₁₋C₄)alkoxyl substituted by a 4- to 6-membered heterocyclic ring, or guanidinyl.

In one instance, R⁴ is (C₁-C₄)alkyl substituted by a 4- to 6-membered heterocyclic ring, or a 5- or 6-membered heteroaryl, wherein said heterocyclic or heteroaryl ring is optionally substituted by one or more (C₁-C₄)alkyl. Examples of R⁴ include (C₁-C₄)alkyl that is substituted by a pyrrolidinyl, azetidinyl, 4-methylpiperazinyl, piperazinyl, imidazolyl, or piperidinyl ring.

Certain compounds of Formula (I) include:

(3S,10S,13S)-10,13-dimethyl-3-(2-(pyrrolidin-1-yl)ethoxy)tetradecahydro-1H-cyclopenta[a]phenanthren-17(2H)-one (Compound 65):

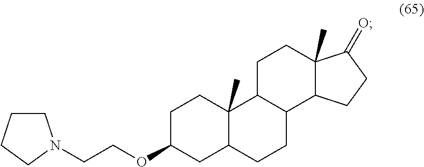

(3S,10S,13S)-10,13-dimethyl-3-(2-(piperidin-1-yl)ethoxy)tetradecahydro-1H-cyclopenta[a]phenanthren-17(2H)-one (Compound 66):

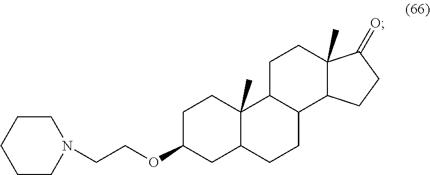

(3S,10S,13S)-10,13-dimethyl-3-(2-(4-methylpiperazin-1-yl)ethoxy)tetradecahydro-1H-cyclopenta[a]phenanthren-17(2H)-one (Compound 67):

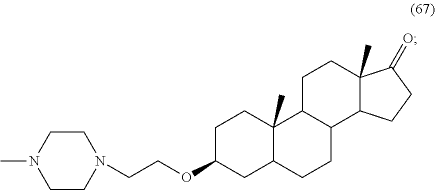

(3S,10S,13S)-3-(2-(1H-imidazol-1-yl)ethoxy)-10,13-dimethyltetradecahydro-1H-cyclopenta[a]phenanthren-17(2H)-one (Compound 68):

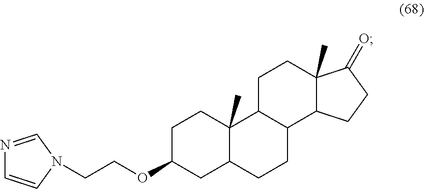

(3S,10S,13S)-3-(3-(1H-imidazol-1-yl)propoxy)-10,13-dimethyltetradecahydro-1H-cyclopenta[a]phenanthren-17(2H)-one (Compound 69):

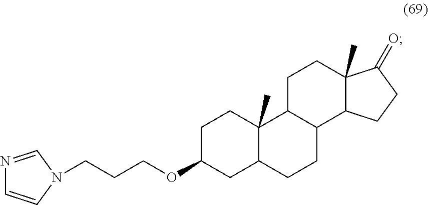

(3S,10R,13S)-10,13-dimethyl-3-(2-(piperidin-1-yl)ethoxy)-2,3,7,8,9,10,11,12,13,14,15,16-dodecahydro-1H-cyclopenta[a]phenanthren-17(6H)-one (Compound 70):

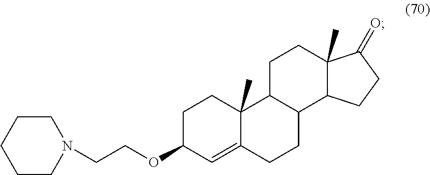

(3S,10R,13S)-10,13-dimethyl-3-(2-(pyrrolidin-1-yl)ethoxy)-2,3,7,8,9,10,11,12,13,14,15,16-dodecahydro-1H-cyclopenta[a]phenanthren-17(6H)-one (Compound 71):

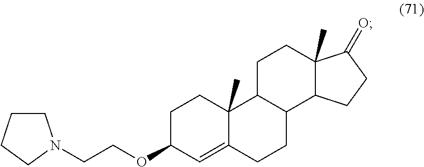

(3S,10R,13S)-10,13-dimethyl-3-(2-(4-methylpiperazin-1-yl)ethoxy)-2,3,7,8,9,10,11,12,13,14,15,16-dodecahydro-1H-cyclopenta[a]phenanthren-17(6H)-one (Compound 72):

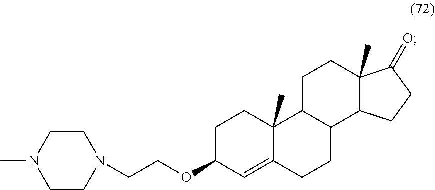

(3S,10R,13S)-3-(3-(1H-imidazol-1-yl)propoxy)-10,13-dimethyl-2,3,7,8,9,10,11,12,13,14,15,16-dodecahydro-1H-cyclopenta[a]phenanthren-17(6H)-one (Compound 73):

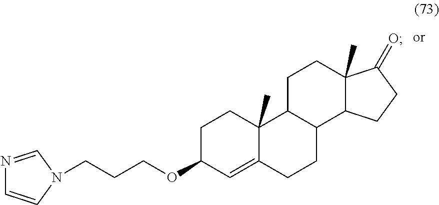

(3S,10R,13S)-3-(2-(1H-imidazol-1-yl)ethoxy)-10,13-dimethyl-2,3,7,8,9,10,11,12,13,14,15,16-dodecahydro-1H-cyclopenta[a]phenanthren-17(6H)-one (Compound 74):

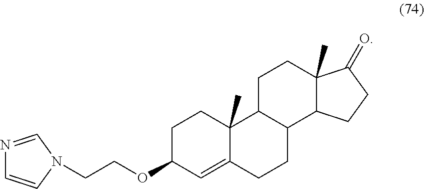

In another instance, R⁴ is (C₁-C₄)alkyl that is optionally substituted by amino(C₁-C₄)alkoxyl, or (C₁-C₄)alkoxyl substituted by a 4- to 6-membered heterocyclic ring. The R⁴ group can be, but is not limited to, (C₁-C₄)alkyl that is substituted by N,N-dimethylamino-1-ethoxyl or pyrrolidinyl-1-ethoxyl. Another instance provides that R⁴ is (C₁-C₄)alkyl substituted by guanidinyl. Certain examples of the compounds of Formula (I) are:

(3S,10S,13S)-3-(2-(2-(dimethylamino)ethoxy)ethoxy)-10,13-dimethyltetradecahydro-1H-cyclopenta[a]phenanthren-17(2H)-one (Compound 75):

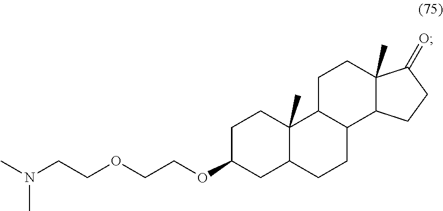

(3S,10S,13S)-10,13-dimethyl-3-(2-(2-(pyrrolidin-1-yl)ethoxy)ethoxy)tetradecahydro-1H-cyclopenta[a]phenanthren-17(2H)-one (Compound 76):

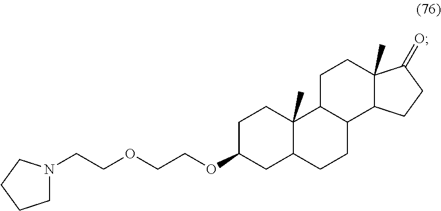

(3S,10R,13S)-10,13-dimethyl-3-(2-(2-(pyrrolidin-1-yl)ethoxy)ethoxy)-2,3,7,8,9,10,11,12,13,14,15,16-dodecahydro-1H-cyclopenta[a]phenanthren-17(6H)-one (Compound 77):

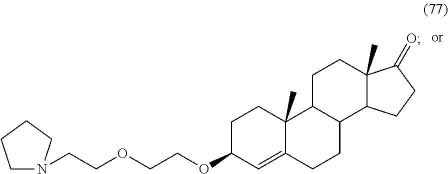

(3S,10R,13S)-3-(2-(2-(dimethylamino)ethoxy)ethoxy)-10,13-dimethyl-2,3,7,8,9,10,11,12,13,14,15,16-dodecahydro-1H-cyclopenta[a]phenanthren-17(6H)-one (Compound 78):

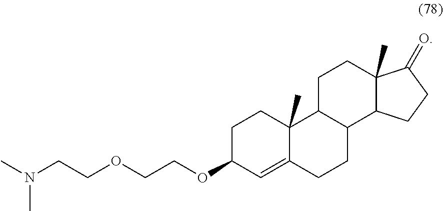

One embodiment provides that A is CH(OR⁴), wherein R⁴ is amino(C₁-C₄)alkyl that is optionally substituted by one or more (C₁-C₄)alkyl. Certain examples of the R⁴ group include aminoethyl, N-methylaminoethyl, N,N-dimethylaminoethyl, 3-aminopropyl, N-methylamino-1-propyl, N,N-dimethylamino-1-propyl, and N,N-dimethylamino-1-butyl.

Compounds of Formula (I) include, but are not limited to,

(3S,10S,13S)-3-(2-aminoethoxy)-10,13-dimethyltetradecahydro-1H-cyclopenta-[a]phenanthren-17(2H)-one (Compound 79):

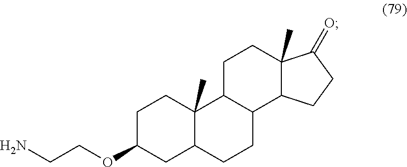

(3S,10S,13S)-10,13-dimethyl-3-(2-(methylamino)ethoxy)tetradecahydro-1H-cyclopenta[a]phenanthren-17(2H)-one (Compound 80):

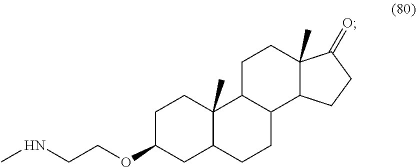

(3S,10S,13S)-3-(3-aminopropoxy)-10,13-dimethyltetradecahydro-1H-cyclopenta-[a]phenanthren-17(2H)-one (Compound 81):

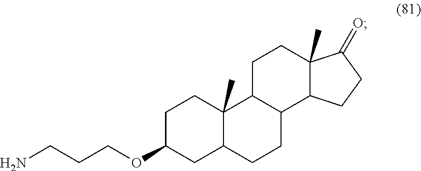

(3S,10S,13S)-10,13-dimethyl-3-(3-(methylamino)propoxy)tetradecahydro-1H-cyclopenta[a]phenanthren-17(2H)-one (Compound 82):

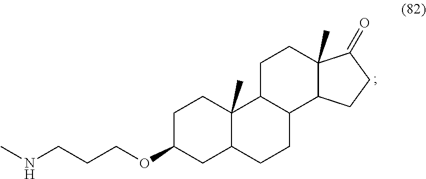

(3S,10S,13S)-3-(3-(dimethylamino)propoxy)-10,13-dimethyltetradecahydro-1H-cyclopenta[a]phenanthren-17(2H)-one (Compound 83):

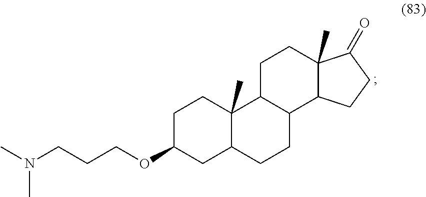

(3R,10S,13S)-3-(3-(dimethylamino)propoxy)-10,13-dimethyltetradecahydro-1H-cyclopenta[a]phenanthren-17(2H)-one (Compound 84):

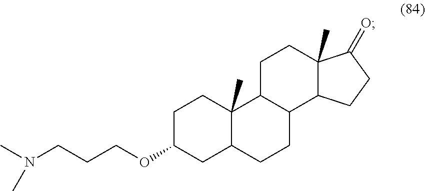

(3S,10S,13S)-3-(4-(dimethylamino)butoxy)-10,13-dimethyltetradecahydro-1H-cyclopenta[a]phenanthren-17(2H)-one (Compound 85):

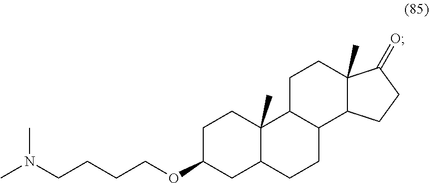

(3S,10R,13S)-3-(2-aminoethoxy)-10,13-dimethyl-2,3,7,8,9,10,11,12,13,14,15,16-dodecahydro-1H-cyclopenta[a]phenanthren-17(6H)-one (Compound 86):

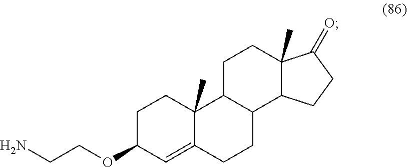

(3S,10R,13S)-10,13-dimethyl-3-(2-(methylamino)ethoxy)-2,3,7,8,9,10,11,12,13,14,15,16-dodecahydro-1H-cyclopenta[a]phenanthren-17(6H)-one (Compound 87):

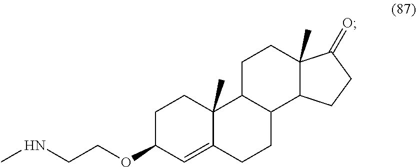

(3S,10R,13S)-3-(2-(dimethylamino)ethoxy)-10,13-dimethyl-2,3,7,8,9,10,11,12,13,14,15,16-dodecahydro-1H-cyclopenta[a]phenanthren-17(6H)-one (Compound 88):

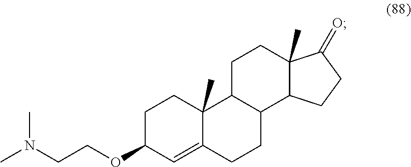

(3S,10R,13S)-3-(3-aminopropoxy)-10,13-dimethyl-2,3,7,8,9,10,11,12,13,14,15,16-dodecahydro-1H-cyclopenta[a]phenanthren-17(6H)-one (Compound 89):

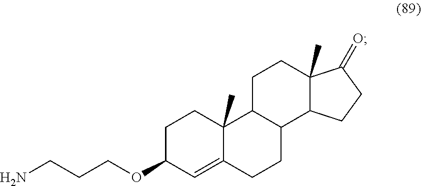

(3S,10R,13S)-10,13-dimethyl-3-(3-(methylamino)propoxy)-2,3,7,8,9,10,11,12,13,14,15,16-dodecahydro-1H-cyclopenta[a]phenanthren-17(6H)-one (Compound 90):

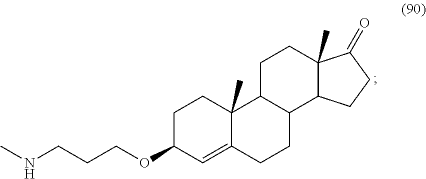

(3S,10R,13S)-3-(3-(dimethylamino)propoxy)-10,13-dimethyl-2,3,7,8,9,10,11,12,13,14,15,16-dodecahydro-1H-cyclopenta[a]phenanthren-17(6H)-one (Compound 91):

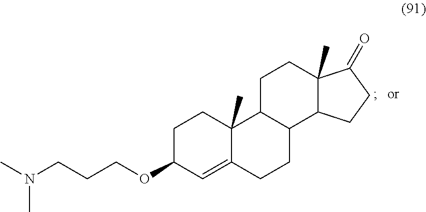

(3S,10R,13S)-3-(4-(dimethylamino)butoxy)-10,13-dimethyl-2,3,7,8,9,10,11,12,13,14,15,16-dodecahydro-1H-cyclopenta[a]phenanthren-17(6H)-one (Compound 92):

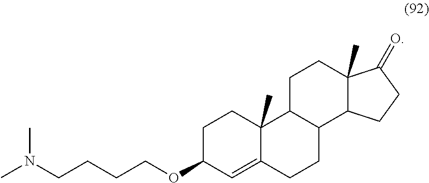

Another embodiment provides that A is CH(OR⁴), wherein R⁴ is amino(C₁-C₄)acyl that is optionally substituted by one or more (C₁-C₄)alkyl. And R⁴ can be, but is not limited to, 3-aminopropanoyl, 3-aminobutanoyl, or aminomethylpropanoyl. Certain compounds of Formula (I) include:

(3S,10S,13S)-10,13-dimethyl-17-oxohexadecahydro-1H-cyclopenta[a]phenanthren-3-yl 3-aminopropanoate (Compound 93):

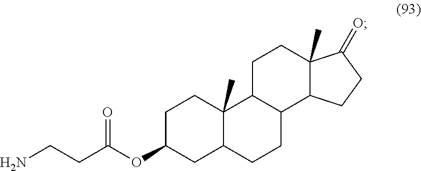

(3S,10S,13S)-10,13-dimethyl-17-oxohexadecahydro-1H-cyclopenta[a]phenanthren-3-yl 3-aminobutanoate (Compound 94):

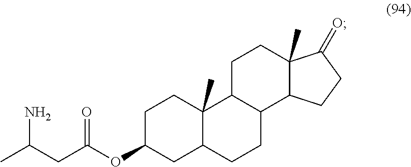

(3S,10S,13S)-10,13-dimethyl-17-oxohexadecahydro-1H-cyclopenta[a]phenanthren-3-yl 3-amino-2-methylpropanoate (Compound 95):

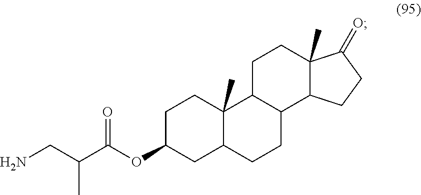

(3S,10R,13S)-10,13-dimethyl-17-oxo-2,3,6,7,8,9,10,11,12,13,14,15,16,17-tetradecahydro-1H-cyclopenta[a]phenanthren-3-yl 3-aminopropanoate (Compound 96):

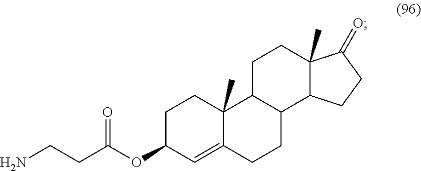

(3S,10R,13S)-10,13-dimethyl-17-oxo-2,3,6,7,8,9,10,11,12,13,14,15,16,17-tetradecahydro-1H-cyclopenta[a]phenanthren-3-yl 3-aminobutanoate (Compound 97):

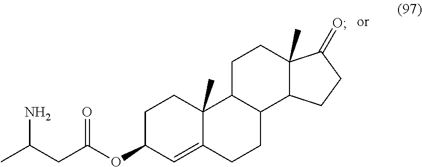

(3S,10R,13S)-10,13-dimethyl-17-oxo-2,3,6,7,8,9,10,11,12,13,14,15,16,17-tetradecahydro-1H-cyclopenta[a]phenanthren-3-yl 3-amino-2-methylpropanoate (Compound 98):

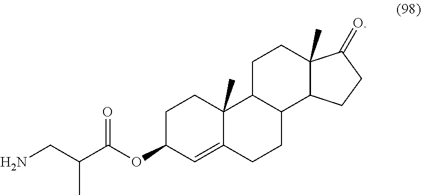

In another embodiment, a compound of Formula (I) has A being CH—CH═N—OR⁶. In one embodiment, R⁶ is amino(C₁-C₄)alkyl that is optionally substituted by one or more (C₁-C₄)alkyl.

A compound of Formula (I) can be, but is not limited to,

(3R,10S,13S)-3-10,13-dimethyl-17-oxohexadecahydro-1H-cyclopenta[a]phenanthrene-3-carbaldehyde O-2-aminoethyl oxime (Compound 99):

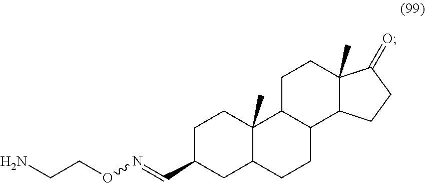

10,13-Dimethyl-17-oxohexadecahydro-1H-cyclopenta[a]phenanthrene-3-carbaldehyde O-2-(dimethylamino)ethyl oxime (Compound 100):

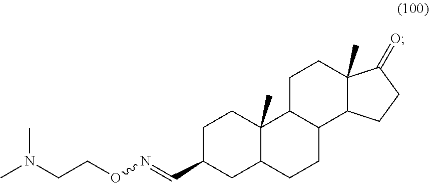

10,13-dimethyl-17-oxo-2,3,6,7,8,9,10,11,12,13,14,15,16,17-tetradecahydro-1H-cyclopenta[a]phenanthrene-3-carbaldehyde O-2-aminoethyl oxime (Compound 101):

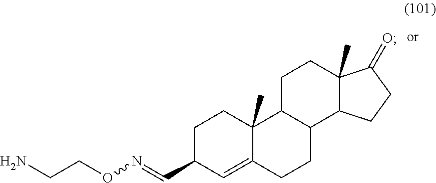

10,13-dimethyl-17-oxo-2,3,6,7,8,9,10,11,12,13,14,15,16,17-tetradecahydro-1H-cyclopenta[a]phenanthrene-3-carbaldehyde O-2-(dimethylamino)ethyl oxime (Compound 102):

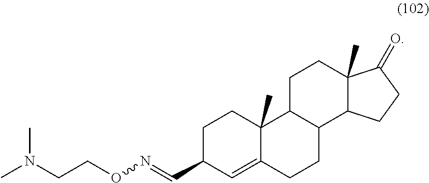

In another embodiment, A in Formula (I) is C═CH—CH═N—OR⁶. Certain compounds of Formula (I) include:

(2E)-2-((10S,13S)-10,13-dimethyl-17-oxohexahydro-1H-cyclopenta[a]phenanthren-3 (2H,4H,10H,12H,13H,14H,15H,16H,17H)-ylidene)acetaldehyde O-2-(dimethylamino) ethyl oxime (Compound 103):

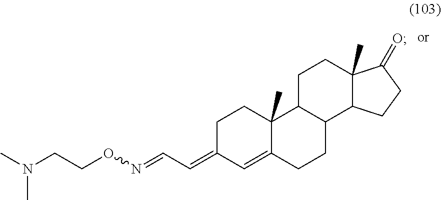

(2E)-2-((10R,13S)-10,13-dimethyl-17-oxo-7,8,9,11-tetrahydro-1H-cyclopenta[a]phenanthren-3(2H,6H,10H,12H,13H,14H,15H,16H,17H)-ylidene)acetaldehyde O-2-(dimethylamino)ethyl oxime (Compound 104):

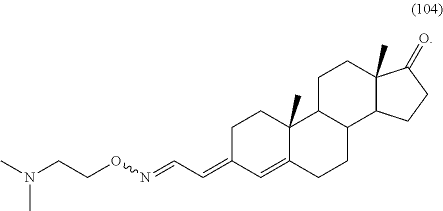

Another embodiment of the invention provides that A is CH—CH═CH—R⁶ in Formula (I), while R⁶ is amino(C₁-C₄)alkyl optionally substituted by one or more (C₁-C₄)alkyl. The R⁶ group can be, but is not limited to, 3-aminopropyl or aminoethyl. Compounds of Formula (I) include, but are not limited to:

(3R,10S,13S)-3-((E)-5-aminopent-1-enyl)-10,13-dimethyltetradecahydro-1H-cyclopenta[a]phenanthren-17(2H)-one (Compound 105):

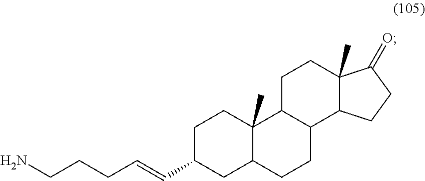

(3S,10S,13S)-3-((E)-4-aminobut-1-enyl)-10,13-dimethyltetradecahydro-1H-cyclopenta[a]phenanthren-17(2H)-one (Compound 106):

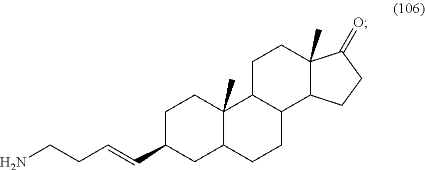

(3S,10S,13S)-3-((E)-5-aminopent-1-enyl)-10,13-dimethyltetradecahydro-1H-cyclopenta[a]phenanthren-17(2H)-one (Compound 107):

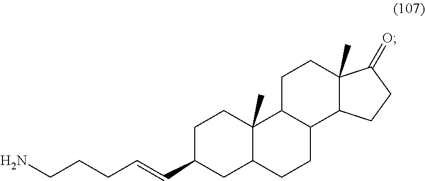

(3S,10R,13S)-3-((E)-5-aminopent-1-enyl)-10,13-dimethyl-2,3,7,8,9,10,11,12,13,14,15,16-dodecahydro-1H-cyclopenta[a]phenanthren-17(6H)-one (Compound 108):

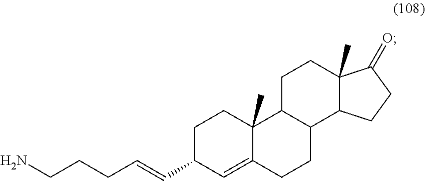

(3R,10R,13S)-3-((E)-4-aminobut-1-enyl)-10,13-dimethyl-2,3,7,8,9,10,11,12,13,14,15,16-dodecahydro-1H-cyclopenta[a]phenanthren-17(6H)-one (Compound 109):

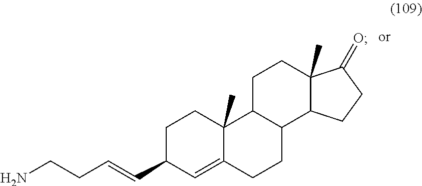

(3R,10R,13S)-3-((E)-5-aminopent-1-enyl)-10,13-dimethyl-2,3,7,8,9,10,11,12,13,14,15,16-dodecahydro-1H-cyclopenta[a]phenanthren-17(6H)-one (Compound 110):

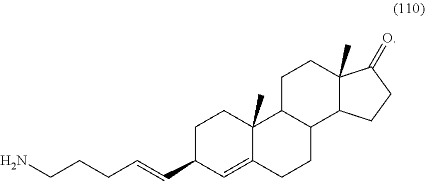

Certain embodiments also provide that A is CH—SR⁷ or CH—S(O)R⁷ in Formula (I). In one instance, R⁷ is 3-aminopropyl. Examples of compounds of Formula (I) are:

(3R,10S,13S)-3-(3-aminopropylthio)-10,13-dimethyltetradecahydro-1H-cyclopenta[a]phenanthren-17(2H)-one (Compound III):

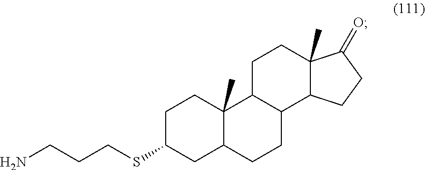

(3R,10S,13S)-3-((S)-3-aminopropylsulfinyl)-10,13-dimethyltetradecahydro-1H-cyclopenta[a]phenanthren-17(2H)-one (Compound 112):

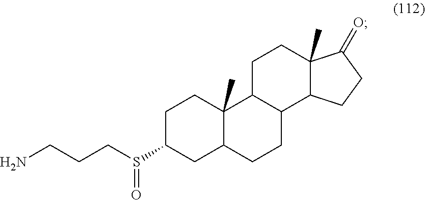

(3R,10R,13S)-3-(3-aminopropylthio)-10,13-dimethyl-2,3,7,8,9,10,11,12,13,14,15,16-dodecahydro-1H-cyclopenta[a]phenanthren-17(6H)-one (Compound 113):

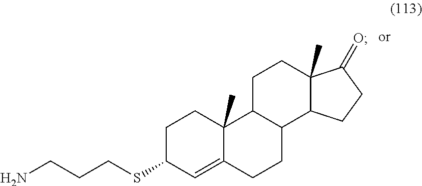

(3R,10R,13S)-3-((R)-3-aminopropylsulfinyl)-10,13-dimethyl-2,3,7,8,9,10,11,12,13,14,15,16-dodecahydro-1H-cyclopenta[a]phenanthren-17(6H)-one (Compound 114):

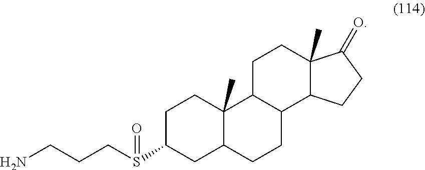

One embodiment provides a compound of Formula (I), wherein one of R¹ and R² is H, and the other is —OR³. In one instance, one of R¹ and R² is H, and the other is —OH.

In one embodiment, A in Formula (I) is C═N—OR⁵.

In one instance, R⁵ is amino(C₁-C₄)alkyl that is optionally substituted by one or more (C₁-C₄)alkyl. And R⁵ can be, but is not limited to, aminoethyl, 2-aminopropyl, 2-methyl-2-aminopropyl, 2-aminomethyl-2-propyl, N-methylamino-ethyl, 3-aminopropyl, N-methylamino-propyl, aminobutyl, N-ethylamino-butyl, N,N-dimethylamino-ethyl, or N-acetylamino-ethyl. Specific examples of R⁵ include N,N-dimethylamino-ethyl, aminoethyl, and 3-aminopropyl.

Certain compounds of Formula (I) include:

(1S,9aS,11aS)-17-hydroxy-10,13-dimethyltetradecahydro-1H-cyclopenta[a]phenanthren-3(2H)-one O-2-(dimethylamino)ethyl oxime (Compound 115):

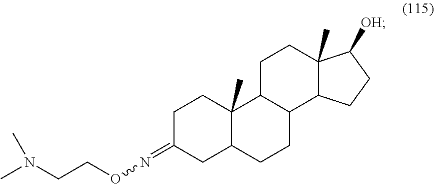

(1S,9aS,11aS)-17-hydroxy-10,13-dimethyltetradecahydro-1H-cyclopenta[a]phenanthren-3(2H)-one O-2-aminoethyl oxime (Compound 116):

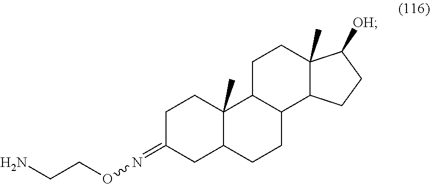

(1R,9aS,11aS)-17-hydroxy-10,13-dimethyltetradecahydro-1H-cyclopenta[a]phenanthren-3(2H)-one O-2-aminoethyl oxime (Compound 117):

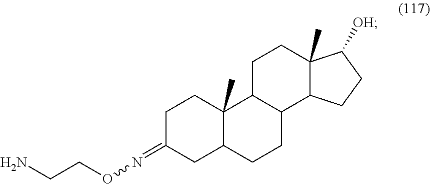

(1S,9aS,11aS)-17-hydroxy-10,13-dimethyltetradecahydro-1H-cyclopenta[a]phenanthren-3(2H)-one O-3-aminopropyl oxime (Compound 118):

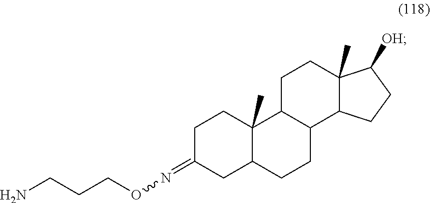

(1R,9aS,11aS)-17-hydroxy-10,13-dimethyltetradecahydro-1H-cyclopenta [a]phenanthren-3(2H)-one O-3-aminopropyl oxime (Compound 119):

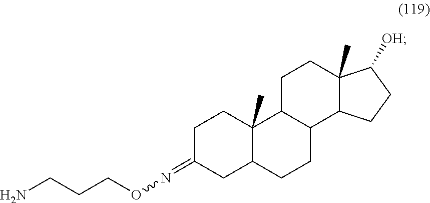

(1S,9aR,11aS)-17-hydroxy-10,13-dimethyl-6,7,8,9,10,11,12,13,14,15,16,17-dodecahydro-1H-cyclopenta[a]phenanthren-3(2H)-one O-2-aminoethyl oxime (Compound 120):

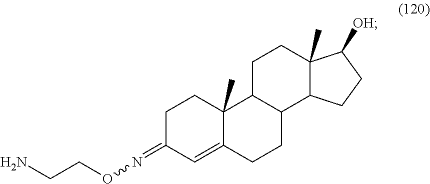

(1S,9aR,11aS)-17-hydroxy-10,13-dimethyl-6,7,8,9,10,11,12,13,14,15,16,17-dodecahydro-1H-cyclopenta[a]phenanthren-3(2H)-one O-3-aminopropyl oxime (Compound 121):

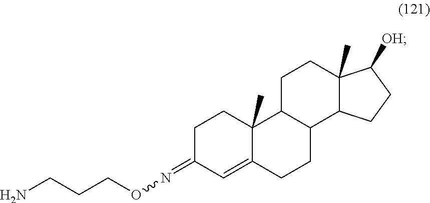

(1R,9aR,11aS)-17-hydroxy-10,13-dimethyl-6,7,8,9,10,11,12,13,14,15,16,17-dodecahydro-1H-cyclopenta[a]phenanthren-3(2H)-one O-2-aminoethyl oxime (Compound 122):

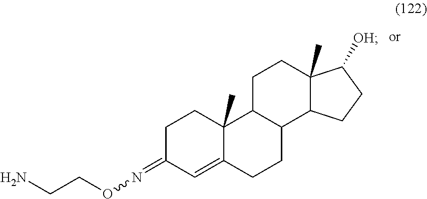

(1R,9aR,11aS)-17-hydroxy-10,13-dimethyl-6,7,8,9,10,11,12,13,14,15,16,17-dodecahydro-1H-cyclopenta[a]phenanthren-3(2H)-one O-3-aminopropyl oxime (Compound 123):

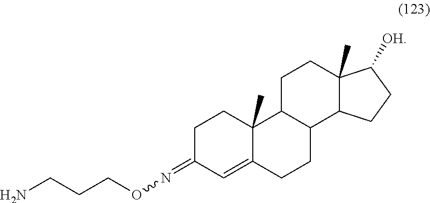

Another embodiment provides that A is C═N—OR⁵, and R⁵ is (C₁-C₄)alkyl that is optionally substituted by guanidinyl, a 5- to 14-membered heteroaryl, or a 4- to 6-membered heterocyclic ring, wherein said heteroaryl is optionally substituted by one or more (C₁-C₄)alkyl or amino(C₁-C₄)alkyl, and said heterocyclic ring is optionally substituted by one or more (C₁-C₄)alkyl.

In one embodiment, R⁵ is (C₁-C₄)alkyl that is substituted by a 5- or 6-membered heteroaryl, wherein said heteroaryl is substituted by amino(C₁-C₄)alkyl that is optionally substituted by one or more (C₁-C₄)alkyl. In one embodiment, said 5- or 6-membered heteroaryl is a triazolyl ring, which is optionally substituted by aminomethyl, aminoethyl, N,N-dimethylaminomethyl, or N,N-dimethylaminoethyl. Certain compounds of Formula (I) include:

(1S,9aS,11aS)-17-hydroxy-10,13-dimethyltetradecahydro-1H-cyclopenta[a]phenanthren-3(2H)-one O-2-(4-(aminomethyl)-1H-1,2,3-triazol-1-yl)ethyl oxime (Compound 124):

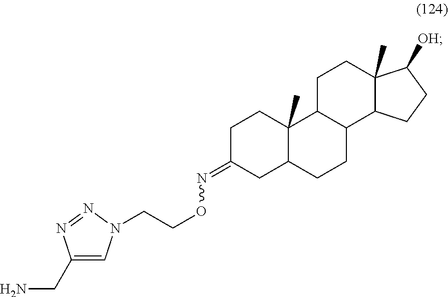

(1S,9aS,11aS)-17-hydroxy-10,13-dimethyltetradecahydro-1H-cyclopenta[a]phenanthren-3(2H)-one O-2-(4-((dimethylamino)methyl)-1H-1,2,3-triazol-1-yl)ethyl oxime (Compound 125):

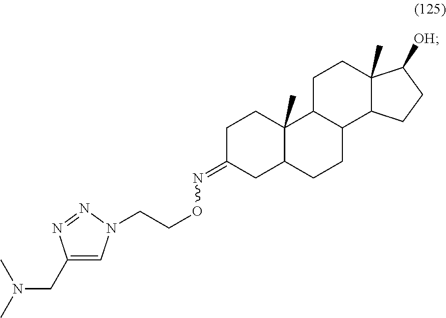

(1S,9aS,11aS)-17-hydroxy-10,13-dimethyltetradecahydro-1H-cyclopenta[a]phenanthren-3(2H)-one O-2-(4-(2-amino ethyl)-1H-1,2,3-triazol-1-yl)ethyl oxime (Compound 126):

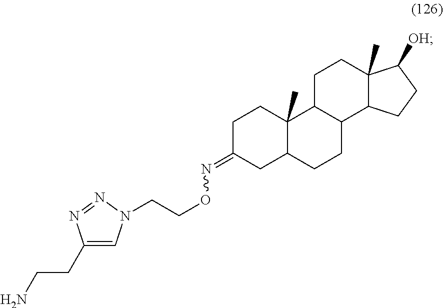

(1S,9aS,11aS)-17-hydroxy-10,13-dimethyltetradecahydro-1H-cyclopenta[a]phenanthren-3(2H)-one O-2-(4-(2-(dimethylamino)ethyl)-1H-1,2,3-triazol-1-yl)ethyl oxime (Compound 127):

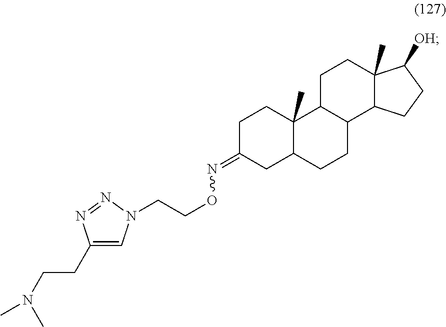

(1S,9aR,11aS)-17-hydroxy-10,13-dimethyl-6,7,8,9,10,11,12,13,14,15,16,17-dodecahydro-1H-cyclopenta[a]phenanthren-3(2H)-one O-2-(4-(aminomethyl)-1H-1,2,3-triazol-1-yl)ethyl oxime (Compound 128):

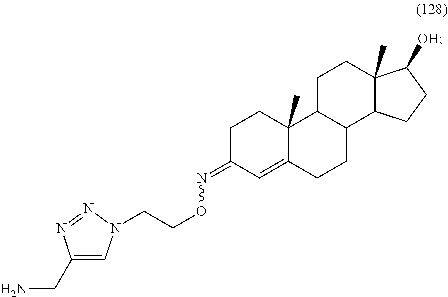

(1S,9aR,11aS)-17-hydroxy-10,13-dimethyl-6,7,8,9,10,11,12,13,14,15,16,17-dodecahydro-1H-cyclopenta[a]phenanthren-3(2H)-one O-2-(4-((dimethylamino)methyl)-1H-1,2,3-triazol-1-yl)ethyl oxime (Compound 129):

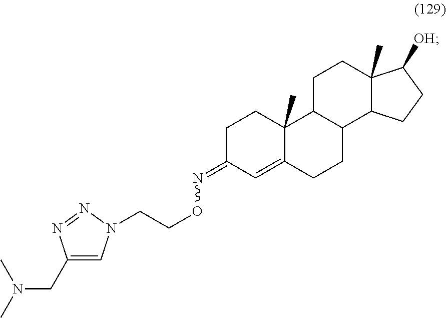

(1S,9aR,11aS)-17-hydroxy-10,13-dimethyl-6,7,8,9,10,11,12,13,14,15,16,17-dodecahydro-1H-cyclopenta[a]phenanthren-3(2H)-one O-2-(4-(2-aminoethyl)-1H-1,2,3-triazol-1-yl)ethyl oxime (Compound 130):

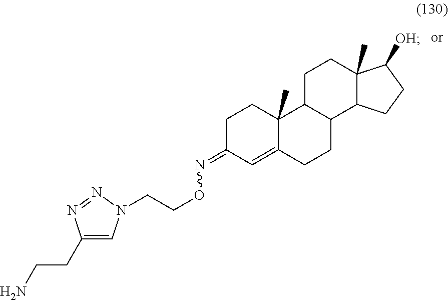

(1S,9aR,11aS)-17-hydroxy-10,13-dimethyl-6,7,8,9,10,11,12,13,14,15,16,17-dodecahydro-1H-cyclopenta[a]phenanthren-3(2H)-one O-2-(4-(2-(dimethylamino)ethyl)-1H-1,2,3-triazol-1-yl)ethyl oxime (Compound 131):

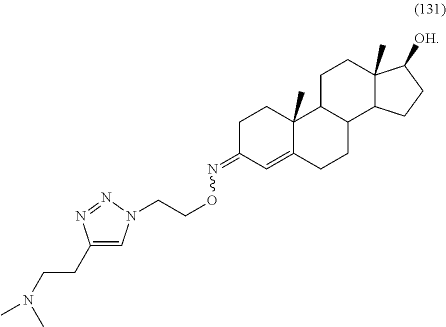

In another embodiment, R⁵ is (C₁-C₄)alkyl that is substituted by an isoxazolyl or oxazolyl ring, which is substituted by (C₁-C₄)alkyl or amino(C₁-C₄)alkyl. Examples of compounds of Formula (I) are:

(1S,9aS,11aS)-17-hydroxy-10,13-dimethyltetradecahydro-1H-cyclopenta [a]phenanthren-3(2H)-one O-2-(5-methylisoxazol-3-yl)ethyl oxime (Compound 132):

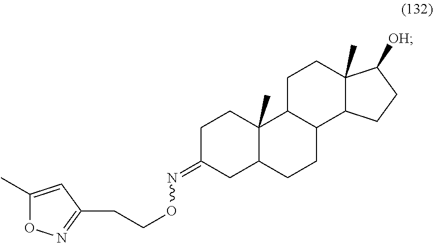

(1S,9aS,11aS)-17-hydroxy-10,13-dimethyltetradecahydro-1H-cyclopenta[a]phenanthren-3(2H)-one O-2-(5-(aminomethyl)isoxazol-3-yl)ethyl oxime (Compound 133):

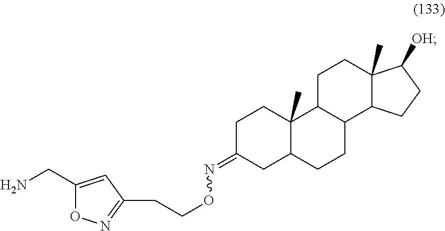

(1S,9aS,11aS)-17-hydroxy-10,13-dimethyltetradecahydro-1H-cyclopenta[a]phenanthren-3(2H)-one O-2-(2-methyloxazol-5-yl)ethyl oxime (Compound 134):

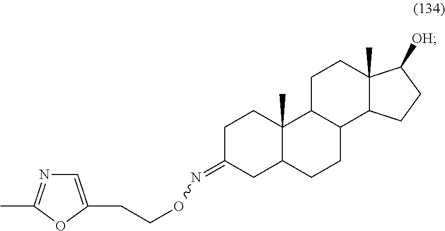

(1S,9aS,11aS)-17-hydroxy-10,13-dimethyltetradecahydro-1H-cyclopenta[a]phenanthren-3(2H)-one O-2-(2-((dimethylamino)methyl)oxazol-5-yl)ethyl oxime (Compound 135):

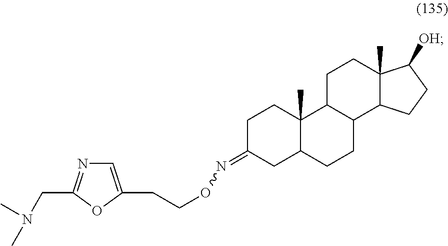

(1S,9aS,11aS)-17-hydroxy-10,13-dimethyltetradecahydro-1H-cyclopenta[a]phenanthren-3(2H)-one O-2-(2-(aminomethyl)oxazol-5-yl)ethyl oxime (Compound 136):

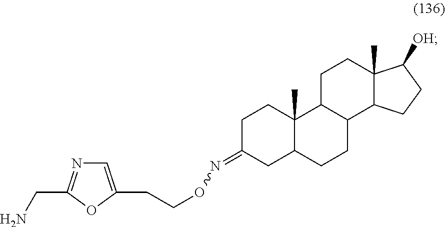

(1S,9aS,11aS)-17-hydroxy-10,13-dimethyltetradecahydro-1H-cyclopenta[a]phenanthren-3(2H)-one O-2-(5-methyloxazol-2-yl)ethyl oxime (Compound 137):

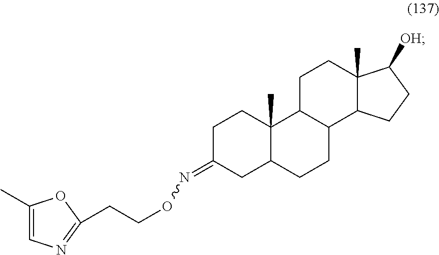

(1S,9aS,11aS)-17-hydroxy-10,13-dimethyltetradecahydro-1H-cyclopenta[a]phenanthren-3(2H)-one O-2-(5-(aminomethyl)oxazol-2-yl)ethyl oxime (Compound 138):

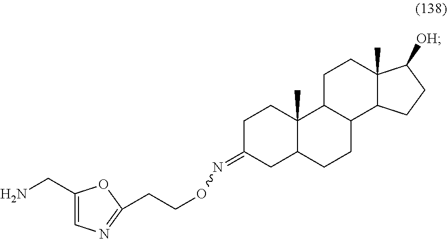

(1S,9aS,11aS)-17-hydroxy-10,13-dimethyltetradecahydro-1H-cyclopenta[a]phenanthren-3(2H)-one O-2-(5-((dimethylamino)methyl)oxazol-2-yl)ethyl oxime (Compound 139):

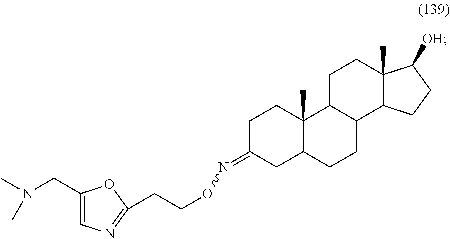

(1S,9aR,11aS)-17-hydroxy-10,13-dimethyl-6,7,8,9,10,11,12,13,14,15,16,17-dodecahydro-1H-cyclopenta[a]phenanthren-3(2H)-one O-2-(5-methylisoxazol-3-yl)ethyl oxime (Compound 140):

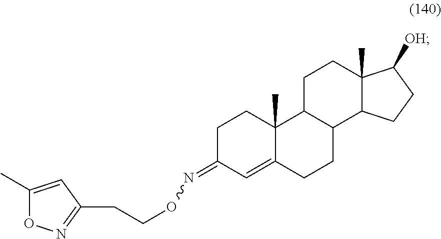

(1S,9aR,11aS)-17-hydroxy-10,13-dimethyl-6,7,8,9,10,11,12,13,14,15,16,17-dodecahydro-1H-cyclopenta[a]phenanthren-3(2H)-one O-2-(5-(aminomethyl)isoxazol-3-yl)ethyl oxime (Compound 141):

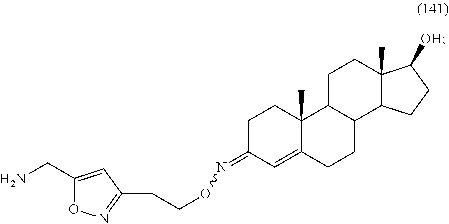

(1S,9aR,11aS)-17-hydroxy-10,13-dimethyl-6,7,8,9,10,11,12,13,14,15,16,17-dodecahydro-1H-cyclopenta[a]phenanthren-3(2H)-one O-2-(2-methyloxazol-5-yl)ethyl oxime (Compound 142):

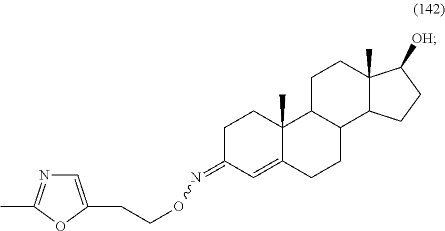

(1S,9aR,11aS)-17-hydroxy-10,13-dimethyl-6,7,8,9,10,11,12,13,14,15,16,17-dodecahydro-1H-cyclopenta[a]phenanthren-3(2H)-one O-2-(2-(aminomethyl)oxazol-5-yl)ethyl oxime (Compound 143):

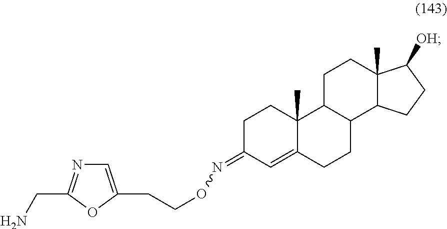

(1S,9aR,11aS)-17-hydroxy-10,13-dimethyl-6,7,8,9,10,11,12,13,14,15,16,17-dodecahydro-1H-cyclopenta[a]phenanthren-3(2H)-one O-2-(2-((dimethylamino)methyl)oxazol-5-yl)ethyl oxime (Compound 144):

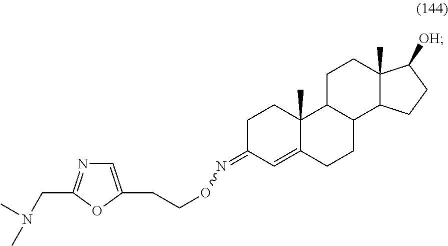

(1S,9aR,11aS)-17-hydroxy-10,13-dimethyl-6,7,8,9,10,11,12,13,14,15,16,17-dodecahydro-1H-cyclopenta[a]phenanthren-3(2H)-one O-2-(5-methyloxazol-2-yl)ethyl oxime (Compound 145):

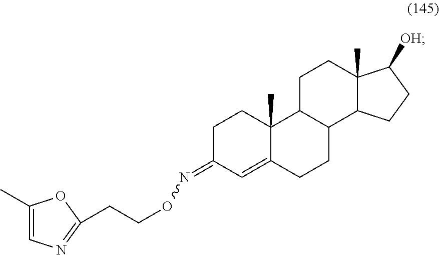

(1S,9aR,11aS)-17-hydroxy-10,13-dimethyl-6,7,8,9,10,11,12,13,14,15,16,17-dodecahydro-1H-cyclopenta[a]phenanthren-3(2H)-one O-2-(5-(aminomethyl)oxazol-2-yl)ethyl oxime (Compound 146):

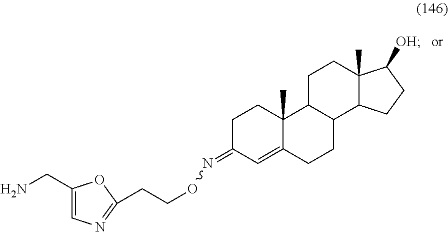

(1S,9aR,11aS)-17-hydroxy-10,13-dimethyl-6,7,8,9,10,11,12,13,14,15,16,17-dodecahydro-1H-cyclopenta[a]phenanthren-3(2H)-one O-2-(5-((dimethylamino)methyl)oxazol-2-yl)ethyl oxime (Compound 147):

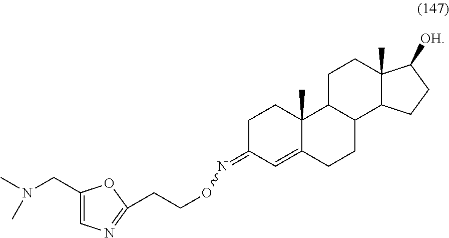

Another embodiment provide that R⁵ is (C₁-C₄)alkyl substituted by an oxadiazolyl ring, which is substituted by (C₁-C₄)alkyl or amino(C₁-C₄)alkyl. A compound of Formula (I) can be, but is not limited to:

(1S,9aS,11aS)-17-hydroxy-10,13-dimethyltetradecahydro-1H-cyclopenta[a]phenanthren-3(2H)-one O-2-(3-methyl-1,2,4-oxadiazol-5-yl)ethyl oxime (Compound 148):

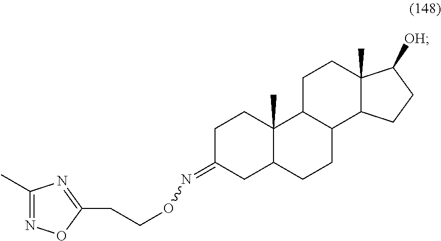

(1S,9aS,11aS)-17-hydroxy-10,13-dimethyltetradecahydro-1H-cyclopenta[a]phenanthren-3(2H)-one O-2-(3-(aminomethyl)-1,2,4-oxadiazol-5-yl)ethyl oxime (Compound 149):

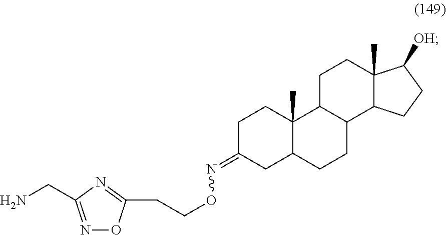

(1S,9aS,11aS)-17-hydroxy-10,13-dimethyltetradecahydro-1H-cyclopenta[a]phenanthren-3(2H)-one O-2-(3-((dimethylamino)methyl)-1,2,4-oxadiazol-5-yl)ethyl oxime (Compound 150):

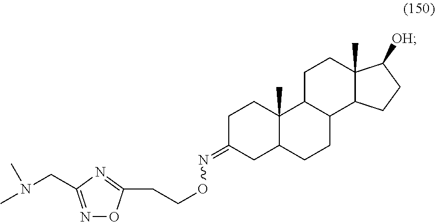

(1S,9aR,11aS)-17-hydroxy-10,13-dimethyl-6,7,8,9,10,11,12,13,14,15,16,17-dodecahydro-1H-cyclopenta[a]phenanthren-3(2H)-one O-2-(3-methyl-1,2,4-oxadiazol-5-yl)ethyl oxime (Compound 151):

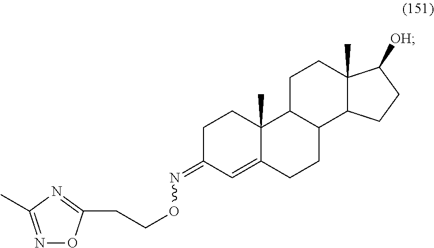

(1S,9aR,11aS)-17-hydroxy-10,13-dimethyl-6,7,8,9,10,11,12,13,14,15,16,17-dodecahydro-1H-cyclopenta[a]phenanthren-3(2H)-one O-2-(3-(aminomethyl)-1,2,4-oxadiazol-5-yl)ethyl oxime (Compound 152):

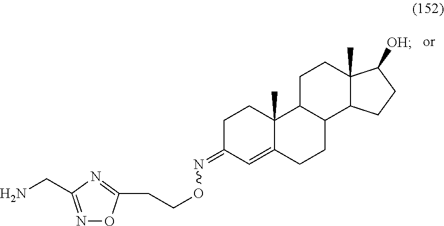

(1S,9aR,11aS)-17-hydroxy-10,13-dimethyl-6,7,8,9,10,11,12,13,14,15,16,17-dodecahydro-1H-cyclopenta[a]phenanthren-3(2H)-one O-2-(3-((dimethylamino)methyl)-1,2,4-oxadiazol-5-yl)ethyl oxime (Compound 153):

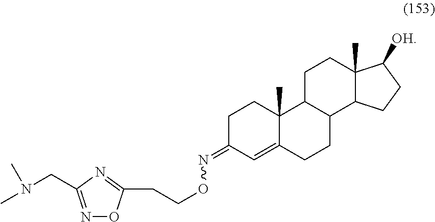

In another embodiment, while one of R¹ and R² is H and the other is —OH, A is CH(OR⁴). In one embodiment, R⁴ is (C₁-C₄)alkyl optionally substituted by a 4- to 6-membered heterocyclic ring, a 5 to 14-membered heteroaryl, amino(C₁₋C₄)alkoxyl, (C₁₋C₄)alkoxyl substituted by a 4- or 6-membered heterocyclic ring, or guanidinyl; wherein said heterocyclic or heteroaryl ring is further optionally substituted by one or more (C₁-C₄)alkyl. One example provides that R⁴ is (C₁-C₄)alkyl substituted by guanidinyl. Certain compounds of Formula (I) include:

1-(2-((3S,10S,13S,17S)-17-hydroxy-10,13-dimethylhexadecahydro-1H-cyclopenta[a]phenanthren-3-yloxy)ethyl)guanidine (Compound 154):

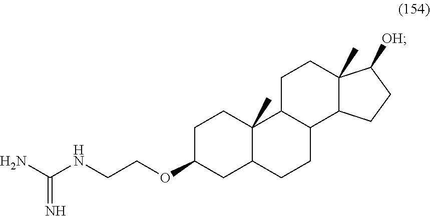

1-(3-((3S,10S,13S,17S)-17-hydroxy-10,13-dimethylhexadecahydro-1H-cyclopenta[a]phenanthren-3-yloxy)propyl)guanidine (Compound 155):

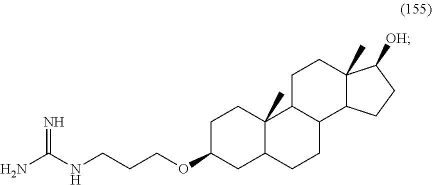

1-(2-((3S,10R,13S,17S)-17-hydroxy-10,13-dimethyl-2,3,6,7,8,9,10,11,12,13,14,15,16,17-tetradecahydro-1H-cyclopenta[a]phenanthren-3-yloxy)ethyl)guanidine (Compound 156):

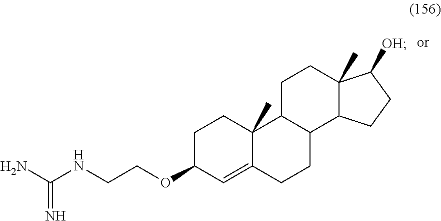

1-(3-((3S,10R,13S,17S)-17-hydroxy-10,13-dimethyl-2,3,6,7,8,9,10,11,12,13,14,15,16,17-tetradecahydro-1H-cyclopenta[a]phenanthren-3-yloxy)propyl)guanidine (Compound 157):

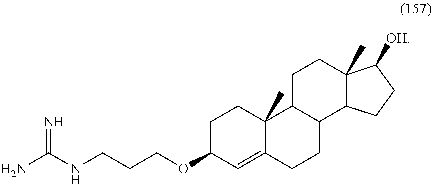

In another embodiment, R⁴ is (C₁-C₄)alkyl that is substituted by a 4- to 6-membered heterocyclic ring. One example of said 4- to 6-membered heterocyclic ring is a pyrrolidinyl ring. Certain compounds of Formula (I) include:

(3S,10S,13S,17S)-10,13-dimethyl-3-(2-(pyrrolidin-1-yl)ethoxy)hexadecahydro-1H-cyclopenta[a]phenanthren-17-ol (Compound 158):

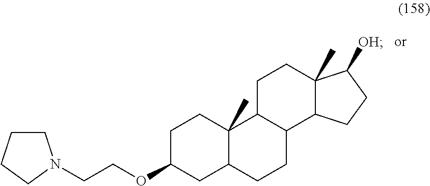

(3S,10R,13S,17S)-10,13-dimethyl-3-(2-(pyrrolidin-1-yl)ethoxy)-2,3,6,7,8,9,10,11,12,13,14,15,16,17-tetradecahydro-1H-cyclopenta[a]phenanthren-17-ol (Compound 159):

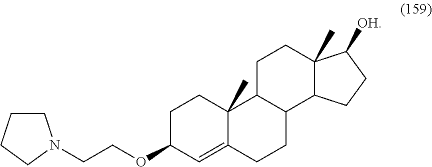

Another embodiment provides that R⁴ is (C₁-C₄)alkyl substituted by amino(C₁-C₄)alkoxyl, which is optionally substituted by one or more (C₁-C₄)alkyl. Examples of the compounds of Formula (I) include:

(3S,10S,13S,17S)-3-(2-(2-(dimethylamino)ethoxy)ethoxy)-10,13-dimethylhexadeca-hydro-1H-cyclopenta[a]phenanthren-17-ol (Compound 160):

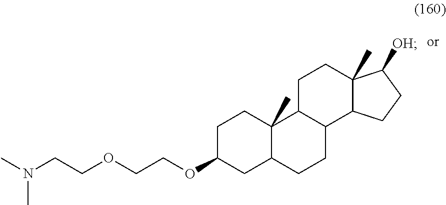

(3S,10R,13S,17S)-3-(2-(2-(dimethylamino)ethoxy)ethoxy)-10,13-dimethyl-2,3,6,7,8,9,10,11,12,13,14,15,16,17-tetradecahydro-1H-cyclopenta[a]phenanthren-17-ol (Compound 161):

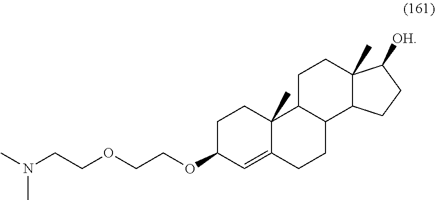

Certain embodiment also provides that R⁴ is (C₁-C₄)alkyl substituted by (C₁-C₄)alkoxyl, which is further substituted by a 4- to 6-membered ring. A compound of Formula (I) can be, but is not limited to,

(3S,10S,13S,17S)-10,13-dimethyl-3-(2-(2-(pyrrolidin-1-yl)ethoxy)ethoxy)hexadeca-hydro-1H-cyclopenta[a]phenanthren-17-ol (Compound 162):

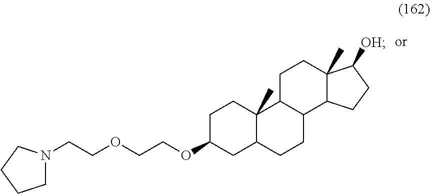

(3S,10R,13S,17S)-10,13-dimethyl-3-(2-(2-(pyrrolidin-1-yl)ethoxy)ethoxy)-2,3,6,7,8,9,10,11,12,13,14,15,16,17-tetradecahydro-1H-cyclopenta[a]phenanthren-17-ol (Compound 163):

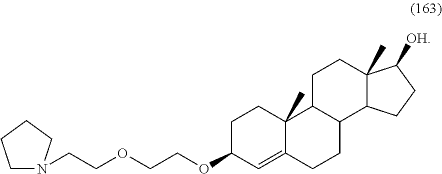

Another embodiment provides that R⁴ is amino(C₁-C₄)alkyl optionally substituted by one or more (C₁-C₄)alkyl. Examples of the R⁴ group include N,N-dimethylamino-ethyl, N,N-dimethylamino-propyl, and N,N-dimethylamino-butyl. Certain compounds of Formula (I) include:

(3S,10S,13S,17S)-3-(2-(dimethylamino)ethoxy)-10,13-dimethylhexadecahydro-1H-cyclopenta[a]phenanthren-17-ol (Compound 164):

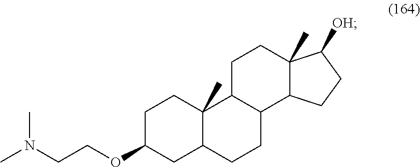

(3S,10S,13S,17S)-3-(3-(dimethylamino)propoxy)-10,13-dimethylhexadecahydro-1H-cyclopenta[a]phenanthren-17-ol (Compound 165):

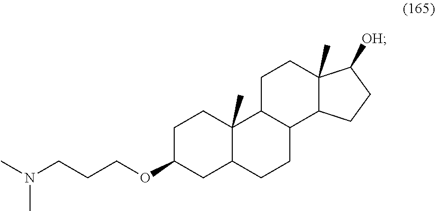

(3S,10S,13S,17S)-3-(4-(dimethylamino)butoxy)-10,13-dimethylhexadecahydro-1H-cyclopenta[a]phenanthren-17-ol (Compound 166):

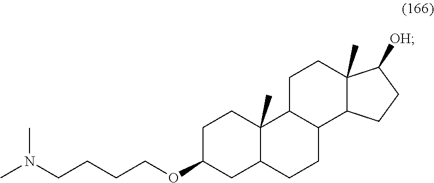

(3R,10S,13S,17S)-3-(3-(dimethylamino)propoxy)-10,13-dimethylhexadecahydro-1H-cyclopenta[a]phenanthren-17-ol (Compound 167):

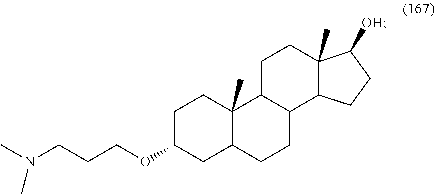

(3S,10R,13S,17S)-3-(2-(dimethylamino)ethoxy)-10,13-dimethyl-2,3,6,7,8,9,10,11,12,13,14,15,16,17-tetradecahydro-1H-cyclopenta[a]phenanthren-17-ol (Compound 168):

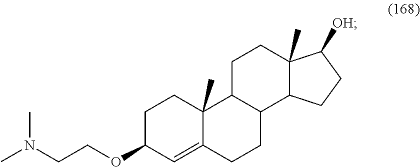

(3S,10R,13S,17S)-3-(3-(dimethylamino)propoxy)-10,13-dimethyl-2,3,6,7,8,9,10,11,12,13,14,15,16,17-tetradecahydro-1H-cyclopenta[a]phenanthren-17-ol (Compound 169):

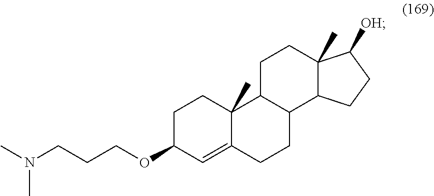

(3S,10R,13S,17S)-3-(4-(dimethylamino)butoxy)-10,13-dimethyl-2,3,6,7,8,9,10,11,12, 13,14,15,16,17-tetradecahydro-1H-cyclopenta[a]phenanthren-17-ol (Compound 170):

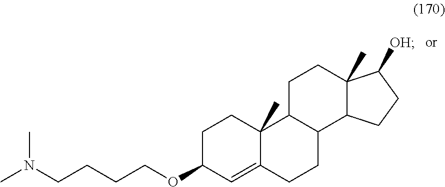

(3R,10R,13S,17S)-3-(3-(dimethylamino)propoxy)-10,13-dimethyl-2,3,6,7,8,9,10,11,12,13,14,15,16,17-tetradecahydro-1H-cyclopenta[a]phenanthren-17-ol (Compound 171):

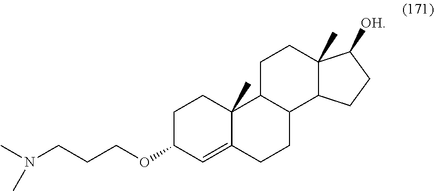

One embodiment provides a compound of Formula (I), wherein one of R¹ and R² is H, and the other is —OH, and A is CH—CH═CH—R⁶. In one embodiment, R⁶ is aminopropyl. In another embodiment, R⁶ is aminoethyl. Certain compounds of Formula (I) include:

(3S,10S,13S,17S)-3-((E)-4-aminobut-1-enyl)-10,13-dimethylhexadecahydro-1H-cyclopenta[a]phenanthren-17-ol (Compound 172):

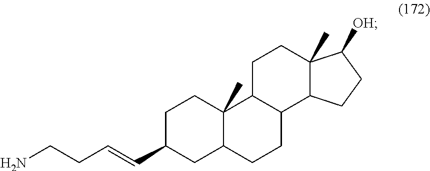

(3R,10S,13S,17S)-3-((E)-5-aminopent-1-enyl)-10,13-dimethylhexadecahydro-1H-cyclopenta[a]phenanthren-17-ol (Compound 173):

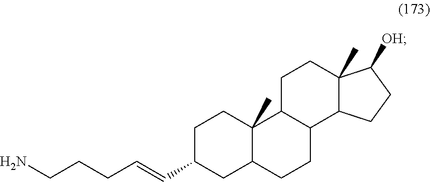

(3R,10R,13S,17S)-3-((E)-4-aminobut-1-enyl)-10,13-dimethyl-2,3,6,7,8,9,10,11,12,13,14,15,16,17-tetradecahydro-1H-cyclopenta[a]phenanthren-17-ol (Compound 174):

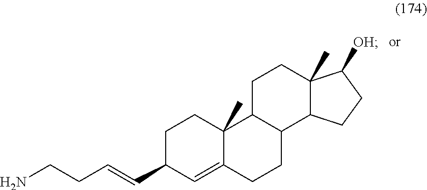

(3S,10R,13S,17S)-3-((E)-5-aminopent-1-enyl)-10,13-dimethyl-2,3,6,7,8,9,10,11,12,13,14,15,16,17-tetradecahydro-1H-cyclopenta[a]phenanthren-17-ol (Compound 175):

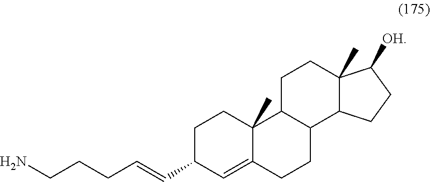

Another embodiment provides a compound of Formula (I), wherein one of R¹ and R² is H, and the other is —OH, and A is CH—CH═N—OR⁶ or C═CH—CH═N—OR⁶. In one embodiment, R⁶ is amino(C₁-C₄)alkyl optionally substituted by one or more (C₁-C₄)alkyl. A compound of Formula (I) can be, but is not limited to:

(2E)-2-((10S,13S,17S)-17-hydroxy-10,13-dimethylhexahydro-1H-cyclopenta-[a]phenanthren-3(2H,4H,10H,12H,13H,14H,15H,16H,17H)-ylidene)acetaldehyde O-2-(dimethylamino)ethyl oxime (Compound 176):

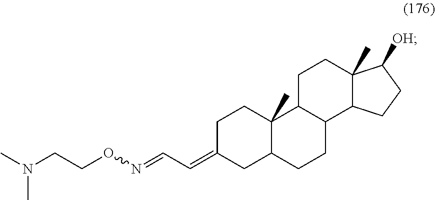

17-hydroxy-10,13-dimethylhexadecahydro-1H-cyclopenta[a]phenanthrene-3-carbaldehyde O-2-(dimethylamino)ethyl oxime (Compound 177):

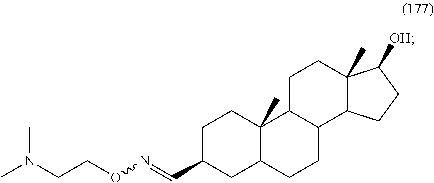

17-hydroxy-10,13-dimethylhexadecahydro-1H-cyclopenta[a]phenanthrene-3-carbaldehyde O-2-aminoethyl oxime (Compound 178):

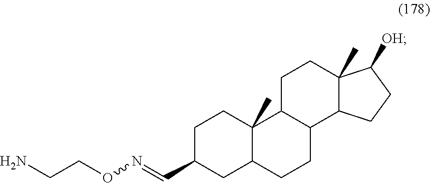

17-hydroxy-10,13-dimethyl-2,3,6,7,8,9,10,11,12,13,14,15,16,17-tetradecahydro-1H-cyclopenta[a]phenanthrene-3-carbaldehyde O-2-aminoethyl oxime (Compound 179):

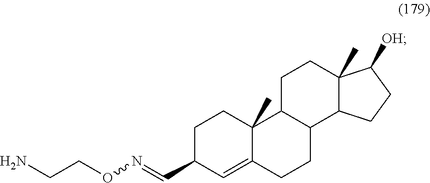

17-hydroxy-10,13-dimethyl-2,3,6,7,8,9,10,11,12,13,14,15,16,17-tetradecahydro-1H-cyclopenta[a]phenanthrene-3-carbaldehyde O-2-(dimethylamino)ethyl oxime (Compound 180):

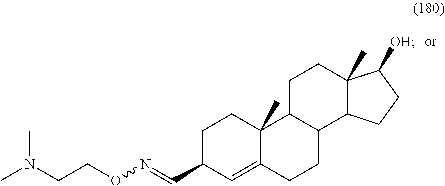

(2E)-2-((10R,13S,17S)-17-hydroxy-10,13-dimethyl-7,8,9,11-tetrahydro-1H-cyclopenta[a]phenanthren-3(2H,6H,10H,12H,13H,14H,15H,16H,17H)-ylidene)acetaldehyde O-2-(dimethylamino)ethyl oxime (Compound 181):

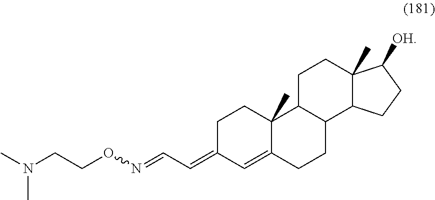

Certain embodiments of the invention also provide a compound of Formula (I), wherein one of R¹ and R² is H, and the other is —OH, and A is CH—SR⁷ or CH—S(O)R⁷. In one embodiment, R⁷ is amino(C₁-C₄)alkyl. Compounds of Formula (I) include:

(3R,10S,13S,17S)-3-(3-aminopropylthio)-10,13-dimethylhexadecahydro-1H-cyclopenta[a]phenanthren-17-ol (Compound 182):

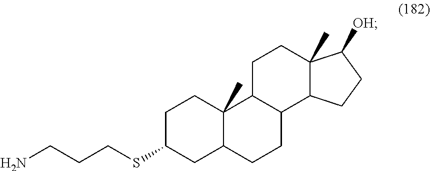

(3R,10S,13S,17S)-3-((S)-3-aminopropylsulfinyl)-10,13-dimethylhexadecahydro-1H-cyclopenta[a]phenanthren-17-ol (Compound 183):

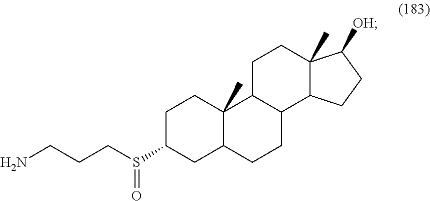

(3R,10R,13S,17S)-3-(3-aminopropylthio)-10,13-dimethyl-2,3,6,7,8,9,10,11,12, 13,14,15,16,17-tetradecahydro-1H-cyclopenta[a]phenanthren-17-ol (Compound 184):

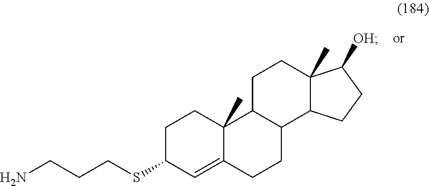

(3R,10R,13S,17S)-3-((R)-3-aminopropylsulfinyl)-10,13-dimethyl-2,3,6,7,8,9,10,11,12,13,14,15,16,17-tetradecahydro-1H-cyclopenta[a]phenanthren-17-ol (Compound 185):

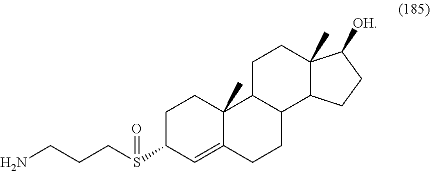

One embodiment of the invention provides a compound of Formula (I), wherein one of R¹ and R² is H, the other is —OR³, and R³ is —NO₂. In one embodiment, A in Formula (I) is C═N—OR⁵. One instance provides that R⁵ is amino(C₁-C₄)alkyl.

A compound of Formula (I) can be, but is not limited to:

(10S,13S,17S)-3-(2-amino ethoxyimino)-10,13-dimethylhexadecahydro-1H-cyclopenta [a]phenanthren-17-yl nitrate (Compound 186):

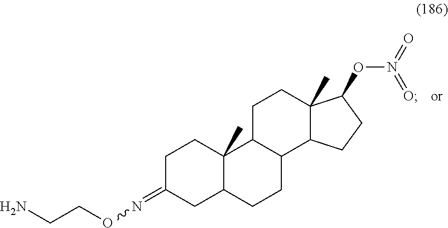

(10R,13S,17S)-3-(2-aminoethoxyimino)-10,13-dimethyl-2,3,6,7,8,9,10,11, 12,13,14, 15,16,17-tetradecahydro-1H-cyclopenta[a]phenanthren-17-yl nitrate (Compound 187):

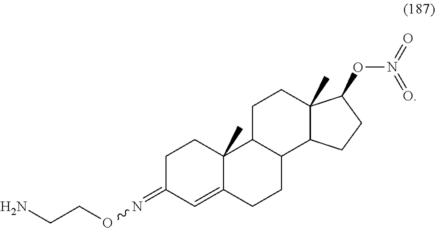

Another embodiment of the invention provides a compound of Formula (I), wherein one of R¹ and R² is H, the other is —OR³, and R³ is carbonyl(C₁-C₄)alkyl. In one embodiment, A is CH(OR⁴). In one instance, R⁴ is amino(C₁-C₄)alkyl optionally substituted by one or more (C₁-C₄)alkyl. Certain compounds of Formula (I) include:

(3S,10S,13S,17S)-3-(2-(dimethylamino)ethoxy)-10,13-dimethylhexadecahydro-1H-cyclopenta[a]phenanthren-17-yl acetate (Compound 188):

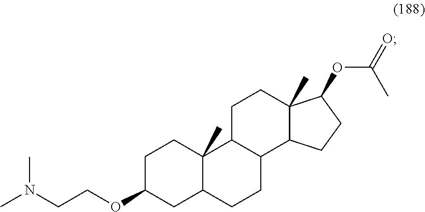

(3S,10S,13S,17S)-3-(3-(dimethylamino)propoxy)-10,13-dimethylhexadecahydro-1H-cyclopenta[a]phenanthren-17-yl acetate (Compound 189):

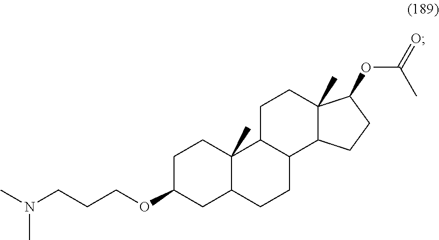

(3S,10R,13S,17S)-3-(2-(dimethylamino)ethoxy)-10,13-dimethyl-2,3,6,7,8,9,10,11,12,13,14,15,16,17-tetradecahydro-1H-cyclopenta[a]phenanthren-17-yl acetate (Compound 190):

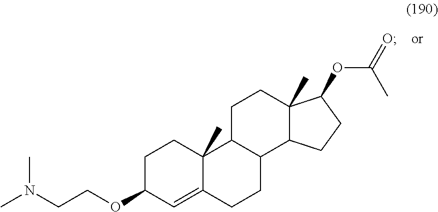

(3S,10R,13S,17S)-3-(3-(dimethylamino)propoxy)-10,13-dimethyl-2,3,6,7,8,9,10,11, 12,13,14,15,16,17-tetradecahydro-1H-cyclopenta[a]phenanthren-17-yl acetate (Compound 191):

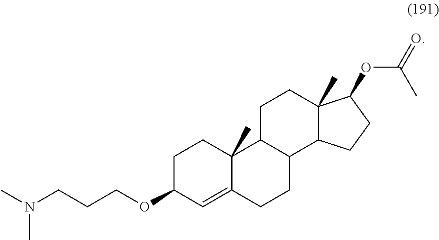

Embodiments of the invention also include a compound of Formula (I), wherein one of R¹ and R² is H, the other is —OR³, and R³ is —C(O)-aryl. Said aryl in the R³ definition can be a 5- to 14-membered aryl group. Examples of the aryl group include benzoyl. In one embodiment, A in Formula (I) is CH(OR⁴). One instance provides a compound of Formula (I), wherein R³ is benzoyl, and R⁴ is amino(C₁-C₄)alkyl that is optionally substituted by one or more (C₁-C₄)alkyl.

A compound in accordance with the invention can be, but is not limited to:

(3S,10S,13S,17S)-3-(3-(dimethylamino)propoxy)-10,13-dimethylhexadecahydro-1H-cyclopenta[a]phenanthren-17-yl benzoate (Compound 192):

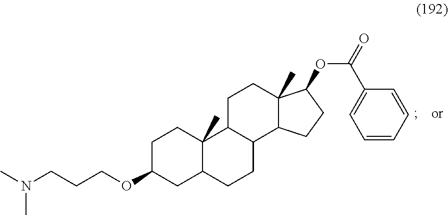

(3S,10R,13S,17S)-3-(3-(dimethylamino)propoxy)-10,13-dimethyl-2,3,6,7,8,9,10,11,12,13,14,15,16,17-tetradecahydro-1H-cyclopenta[a]phenanthren-17-yl benzoate (Compound 193):

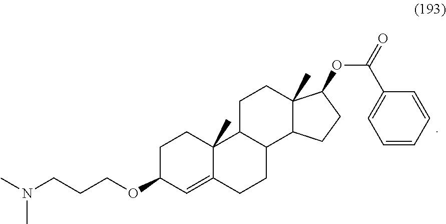

Other embodiments of the invention provide a compound of Formula (I), wherein one of R¹ and R² is H, the other is —OR³, wherein R³ is amino(C₁-C₄)alkyl optionally substituted with one or more (C₁-C₄)alkyl. In one embodiment, A is CH(OR⁴), and R⁴ is amino(C₁-C₄)alkyl optionally substituted by one or more (C₁-C₄)alkyl. Certain compounds of Formula (I) include:

3-((3S,10S,13S,17S)-17-(2-(dimethylamino)ethoxy)-10,13-dimethylhexadecahydro-1H-cyclopenta[a]phenanthren-3-yloxy)-N,N-dimethylpropan-1-amine (Compound 194):

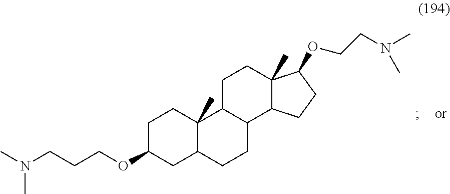

3-((3S,10R,13S,17S)-17-(2-(dimethylamino)ethoxy)-10,13-dimethyl-2,3,6,7,8,9,10,11,12,13,14,15,16,17-tetradecahydro-1H-cyclopenta[a]phenanthren-3-yloxy)-N,N-dimethylpropan-1-amine (Compound 195):

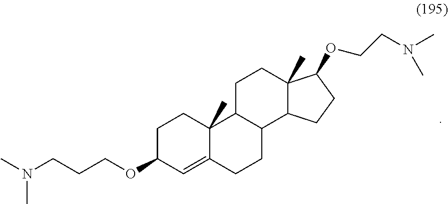

The invention also provides a compound of Formula (Ia):

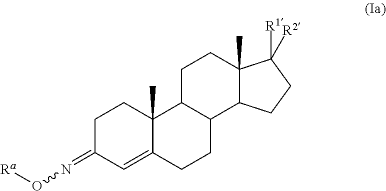

wherein

one of R^{1′} and R^{2′} is H, and the other is —OH; or R^{1′} and R^{2′} together with the carbon to which they are attached form C═O;

R^{a} is aminoalkyl or a 4- to 6-membered heterocyclic ring; wherein said aminoalkyl or said heterocyclic ring is optionally substituted by one or more alkyl.

In one embodiment, a compound of Formula (Ia) has one of R^{1′} and R^{2′} being H, and the other being —OH. In another embodiment, R^{1′} and R^{2′} together with the carbon to which they are attached form C═O.

In a certain embodiment, R^{a} is amino(C₁-C₄)alkyl. In a separate embodiment, R^{a} is a 4- to 6-membered heterocyclic ring. Certain compounds of Formula (Ia) include:

(10R,13S)-3-(2-aminoethoxyimino)-10,13-dimethyl-2,3,7,8,9,10,11,12,13,14,15,16-dodecahydro-1H-cyclopenta[a]phenanthren-17(6H)-one (Compound 12):

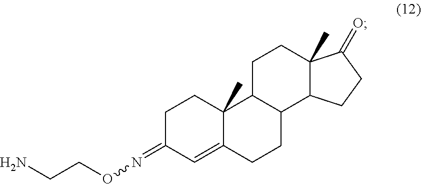

(10R,13S)-10,13-dimethyl-3-(pyrrolidin-3-yloxyimino)-2,3,7,8,9,10,11,12,13,14,15,16-dodecahydro-1H-cyclopenta[a]phenanthren-17(6H)-one (Compound 61):

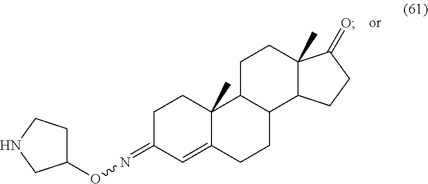

(1S,9aR,11aS)-17-hydroxy-10,13-dimethyl-6,7,8,9,10,11,12,13,14,15,16,17-dodecahydro-1H-cyclopenta[a]phenanthren-3(2H)-one O-2-aminoethyl oxime (Compound 120):

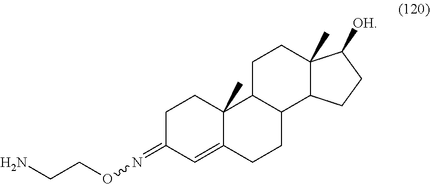

The invention further provides a compound of Formula (Ib):

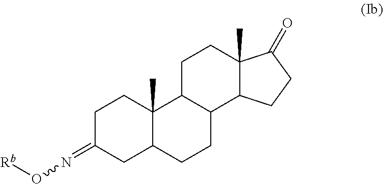

wherein

R^{b} is alkyl, aminoalkyl, or a 4- to 6-membered heterocyclic ring; wherein said alkyl is substituted by a heteroaryl that is further optionally substituted by one or more aminoalkyl; said aminoalkyl, each independently, is optionally substituted by one or more alkyl; and said heterocyclic ring is optionally substituted by one or more alkyl.

In a certain embodiment, R^{b} is (C₁-C₄)alkyl substituted by a heteroaryl that is further substituted by an amino(C₁-C₄)alkyl moiety. One embodiment provides a compound of Formula (Ib) wherein R^{b} is amino(C₁-C₄)alkyl. In another embodiment, R^{b} is a 4- to 6-membered heterocyclic ring.

Examples of compounds of Formula (Ib) include

(10S,13S)-3-(2-aminoethoxyimino)-10,13-dimethyltetradecahydro-1H-cyclopenta[a]phenanthren-17(2H)-one (Compound 1):

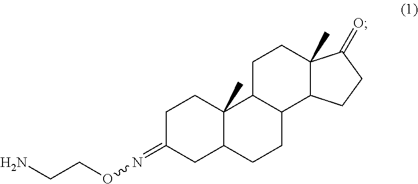

(10S,13S)-10,13-dimethyl-3-(pyrrolidin-3-yloxyimino)tetradecahydro-1H-cyclopenta[a]phenanthren-17(2H)-one (Compound 56):

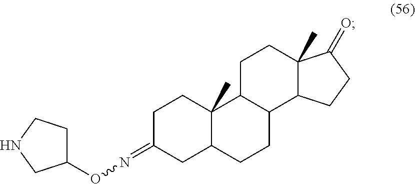

(10S,13S)-3-(2-(4-(aminomethyl)-1H-1,2,3-triazol-1-yl)ethoxyimino)-10,13-dimethyltetradecahydro-1H-cyclopenta[a]phenanthren-17(2H)-one (Compound 31):

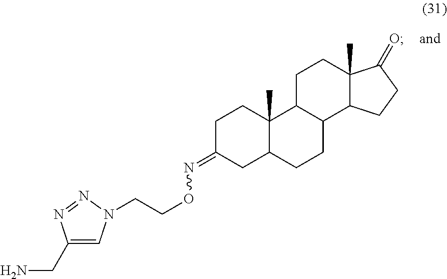

(10S,13S)-3-(2-(4-(aminomethyl)-1H-1,2,3-triazol-1-yl)butoxyimino)-10,13-dimethyltetradecahydro-1H-cyclopenta[a]phenanthren-17(2H)-one (Compound 196):

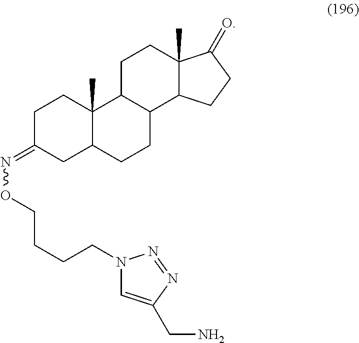

In another aspect, the invention provides a method for reducing the growth, proliferation or survival of a neoplastic cell. The method includes contacting the cell with an effective amount of a compound of the invention. The compound reduces the growth, proliferation or survival of a neoplastic cell.

In one embodiment, the method further comprises selecting the compound for binding to a membrane androgen receptor or for competing with an endogenous ligand for binding to said receptor.

In a separate embodiment, neoplasia is a solid tumor or hematological cancer. Yet another embodiment provides that the neoplastic cell is derived from a tissue selected from the group consisting of lung, breast, CNS, colon, prostate, ovary, pancreas, kidney and melanoma.

In one embodiment, the cell expresses MDR-1 or P-glycoprotein.

Yet another aspect of the invention provides a method of inducing cell death in a neoplastic cell. The method includes contacting the cell with a therapeutically effective amount of a compound of the invention, which thereby induces cell death. In one embodiment, the cell is in a subject. Another embodiment provides that the cell death is apoptotic cell death.

A further aspect of this invention provides a method of preventing or treating a neoplasia in a subject. This method includes administering to a subject in need thereof a therapeutically effective amount of a compound of the invention, which thereby prevents or treats neoplasia in the subject.

One embodiment provides that the subject is a mammal. Another embodiment provides that the subject is a human patient.

In certain embodiments, the methods of the invention reduce the growth or proliferation of a neoplasia in a subject. The neoplasia recited in the methods of the invention can be, but is not limited to, a lung, breast, CNS, colon, prostate, ovary, pancreas, kidney or skin cancer.

In some instances, the neoplasia is resistant to one or more therapeutic agents. In other instances, the neoplasia is multidrug resistant. Certain embodiments provide that the neoplasia has alterations in the expression or activity of an ABC transporter, tubulin, or topoisomerase polypeptide or polynucleotide. In other embodiments, the neoplasia has an increase in the expression or activity of MDR1 or P-glycoprotein.

In another aspect, the invention provides a method for the treatment of a subject having a multidrug resistant or refractory neoplasia. This method includes administering to the subject in need thereof a therapeutically effective amount of a compound of the invention, which thereby treats a subject having a multidrug resistant or refractory neoplasia. In one embodiment, the subject is a human patient. In a separate embodiment, the method reduces the growth or proliferation of the neoplasia. In another embodiment, the method induces the death of a neoplastic cell.

In one embodiment, the neoplasia is resistant to one or more therapeutic agents. In another embodiment, the neoplasia has alterations in the expression or activity of an ABC transporter, tubulin, or topoisomerase polypeptide or polynucleotide. Other embodiments provide that the neoplasia has an increase in the expression or activity of MDR1 or P-glycoprotein.

The method may further comprise administering a compound selected from the group consisting of vinca alkaloids, taxanes, epothilones, antifolates, purine analogs, pyrimidine analogs, DNA intercalators, topoisomerase inhibitors, topotecan, alkylating agents, platinum-based agents, receptor antagonists, hormone agents, anthracyclines, epipodophyllotoxins, antibiotics, antimicrotubule drugs, protein synthesis inhibitors, toxic peptides, enzyme inhibitors and anti-mitotics. The method may also treat a patient having end-stage disease.

This invention also provides a composition for the treatment of a neoplasia. The composition includes a therapeutically effective amount of a compound of the invention and a pharmaceutically acceptable excipient. In one embodiment, the composition further includes a therapeutically effective amount of a chemotherapeutic compound. In another embodiment, the composition further includes a therapeutically effective amount of a taxane.

A further aspect of this invention provides a packaged pharmaceutical for the treatment of neoplasia. The packaged pharmaceutical includes a therapeutically effective amount of a compound of the invention, and written instructions for administration of the compound.

In another aspect, the invention provides a method of preventing or treating a neoplasia (e.g., a membrane androgen positive solid tumor or hematological malignancy) in a subject (e.g., mouse, dog, human) by administering to the subject an effective amount of a compound of the invention. It is believed that a compound of the invention may act as a Na⁺K⁺ ATPase inhibitor that inhibits ligand binding to a membrane androgen receptor, thereby preventing or treating the neoplasia. In one embodiment, the Na⁺K⁺ ATPase inhibitor binds a Na⁺K⁺ ATPase and inhibits Na⁺K⁺ ATPase activity. In another embodiment, the Na⁺K⁺ ATPase inhibitor binds to a membrane androgen receptor and competitively inhibits ligand binding to the receptor. In another embodiment, the Na⁺K⁺ ATPase inhibitor induces cell death in a neoplastic cell of the neoplasia. In still other embodiments, the neoplasia is a prostate cancer, breast cancer, or colon cancer.

In certain embodiments, compounds for use in this aspect of the invention include:

(1S,9aR,11aS)-17-hydroxy-10,13-dimethyl-6,7,8,9,10,11,12,13,14,15,16,17-dodecahydro-1H-cyclopenta[a]phenanthren-3(2H)-one O-2-aminoethyl oxime (Compound 120):

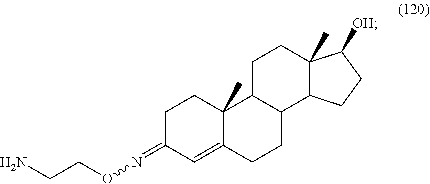

(10R,13S)-3-(2-amino ethoxyimino)-10,13-dimethyl-2,3,7,8,9,10,11,12,13,14,15,16-dodecahydro-1H-cyclopenta[a]phenanthren-17(6H)-one (Compound 12):

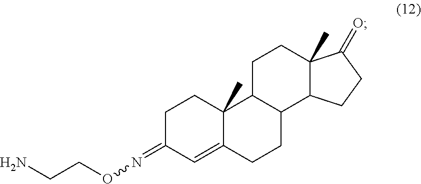

(10R,13S)-10,13-dimethyl-3-(pyrrolidin-3-yloxyimino)-2,3,7,8,9,10,11,12,13,14,15,16-dodecahydro-1H-cyclopenta[a]phenanthren-17(6H)-one (Compound 61):

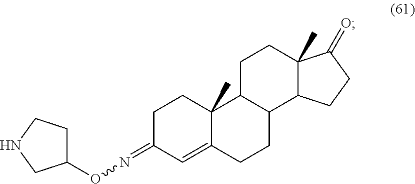

(10S,13S)-3-(2-amino ethoxyimino)-10,13-dimethyltetradecahydro-1H-cyclopenta[a]phenanthren-17(2H)-one (Compound 1):

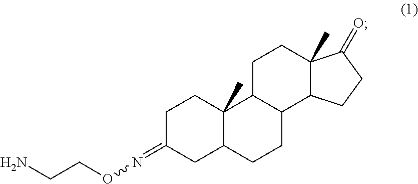

(10S,13S)-10,13-dimethyl-3-(pyrrolidin-3-yloxyimino)tetradecahydro-1H-cyclopenta[a]phenanthren-17(2H)-one (Compound 56):

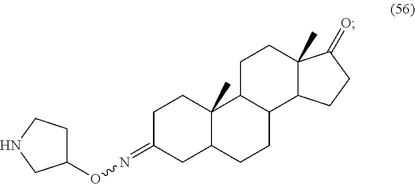

(10S,13S)-3-(2-(4-(aminomethyl)-1H-1,2,3-triazol-1-yl)ethoxyimino)-10,13-dimethyltetradecahydro-1H-cyclopenta[a]phenanthren-17(2H)-one (Compound 31):

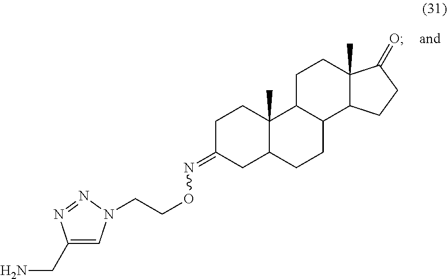

(10S, 13S)-3-(2-(4-(aminomethyl)-1H-1,2,3-triazol-1-yl)butoxyimino)-10,13-dimethyltetradecahydro-1H-cyclopenta[a]phenanthren-17(2H)-one (Compound 196):

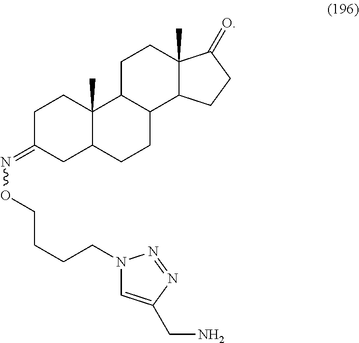

In yet another aspect, the invention provides a method for treating or preventing prostate cancer in a subject. The method involves administering to the subject an effective amount of a compound of the invention that is capable of binding and inhibiting a Na⁺K⁺ ATPase, and further capable of competitively inhibiting ligand binding to the membrane androgen receptor on a prostate cancer cell. In one embodiment, the method induces cell death (e.g., apoptosis) in a cell of the prostate cancer. In another embodiment, the compound binds the membrane androgen receptor. In certain embodiments, compounds for use in this aspect of the invention include compounds 120, 12, 61, 1, 56, 31 and 196 shown above.

The invention further provides methods for treating neoplasia. Compositions and articles defined by the invention were isolated or otherwise manufactured in connection with the examples provided below. Other features and advantages of the invention will be apparent from the detailed description, and from the claims.

### BRIEF DESCRIPTION OF THE DRAWINGS

FIGS. 1A-1E are graphs showing results absorbance of a 3-(4,5-dimethylthiazol-2-yl)-2,5-diphenyltetrazolium bromide (MTT) proliferation/viability assay, which uses the reduction of MTT to formazan as a measure of metabolic activity.

FIGS. 2A-2B are graphs showing that compound 56 has anti-tumor activities in PC-3 xenografts (FIG. 2A) and A549 xenografts (FIG. 2B) respectively.

FIG. 3A, panels A-F, are micrographs showing DAPI and membrane androgen receptor immunofluoresscent staining in LNCaP cells treated with compound 12 relative to control cells. FIG. 3A, panels, B and F show that testosterone-HSA-FITC (TAC-FITC) conjugates showed clear membrane staining as compared to control HAS-FITC conjugates. Cells pre-treated with testosterone 3-(O-carboxymethyl)oxime (Testo-CMO; the steroid moiety present in TAC-FITC conjugates) were devoid of membrane staining

FIG. 3B, panels A-H, are micrographs showing DAPI and membrane androgen receptor immunofluorescent staining in DU145 cells treated with compound 12 relative to control cells. Cells pre-treated with compound 12 were devoid of membrane fluorescence (FIG. 3B: compare panels B and D) indicating that compound 12 precludes binding of TAC-FITC to the membrane androgen receptor.

FIG. 3C, panels A-F, are micrographs showing DAPI and membrane androgen receptor immunofluorescent staining in DU145 cells obtained with TAC-FITC conjugates with or without treatment with compound 56. Cells pre-treated with compound 56 where devoid of membrane fluorescence indicating that the compound precludes binding of TAC-FITC to the membrane androgen receptor.

### DETAILED DESCRIPTION OF THE INVENTION

The invention features compounds delineated herein and methods of using such compounds for the treatment or prevention of neoplasia. In particular embodiments, the compounds of the invention are useful for the treatment of multidrug resistant neoplasia.

The invention is based, at least in part, on the discovery that a compound of the invention has potent anti-neoplastic activity in vitro. In certain instances, a compound of the invention is a Na+/K+ ATPase inhibitor that also has potent anti-neoplastic activity. As shown herein below, Compound Nos. 12, 61, 120, 1, 56, 31 and 196 reduced the viability and/or cell proliferation of cell lines representative of lung, breast, CNS, colon, prostate, ovary, pancreas, kidney and melanoma neoplasias, including a multidrug resistant cell line.

### DEFINITIONS

Before a further description of the present invention, and in order that the invention may be more readily understood, certain terms are first defined and collected here for convenience.

The term “administration” or “administering” includes routes of introducing a compound(s) to a subject to perform their intended function. Examples of routes of administration that can be used include injection (subcutaneous, intravenous, parenterally, intraperitoneally, intrathecal), oral, inhalation, rectal and transdermal. The pharmaceutical preparations are, of course, given by forms suitable for each administration route. For example, these preparations are administered in tablets or capsule form, by injection, inhalation, topical by lotion or ointment; and rectal by suppositories. Oral administration is preferred. The injection can be bolus or can be continuous infusion. Depending on the route of administration, the compound can be coated with or disposed in a selected material to protect it from natural conditions which may detrimentally effect its ability to perform its intended function.

The compound can be administered alone, or in conjunction with either another agent as described above (e.g. another chemotherapeutic agent) or with a pharmaceutically-acceptable carrier, or both. The compound can be administered prior to the administration of the other agent, simultaneously with the agent, or after the administration of the agent. Furthermore, the compound can also be administered in a proform which is converted into its active metabolite, or more active metabolite in vivo.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. The term alkyl further includes alkyl groups, which can further include oxygen, nitrogen, sulfur or phosphorous atoms replacing one or more carbons of the hydrocarbon backbone, e.g., oxygen, nitrogen, sulfur or phosphorous atoms. In preferred embodiments, a straight chain or branched chain alkyl has 30 or fewer carbon atoms in its backbone (e.g., C₁-C₃₀ for straight chain, C₃-C₃₀ for branched chain), preferably 26 or fewer, and more preferably 20 or fewer. Likewise, preferred cycloalkyls have from 3-10 carbon atoms in their ring structure, and more preferably have 3, 4, 5, 6 or 7 carbons in the ring structure.

Moreover, the term alkyl as used throughout the specification and claims is intended to include both “unsubstituted alkyls” and “substituted alkyls,” the latter of which refers to alkyl moieties having substituents replacing a hydrogen on one or more carbons of the hydrocarbon backbone. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. It will be understood by those skilled in the art that the moieties substituted on the hydrocarbon chain can themselves be substituted, if appropriate. Cycloalkyls can be further substituted, e.g., with the substituents described above. An “alkylaryl” moiety is an alkyl substituted with an aryl (e.g., phenylmethyl (benzyl)). The term “alkyl” also includes unsaturated aliphatic groups analogous in length and possible substitution to the alkyls described above, but that contain at least one double or triple bond respectively.

Unless the number of carbons is otherwise specified, “lower alkyl” as used herein means an alkyl group, as defined above, but having from one to ten carbons, more preferably from one to six, and most preferably from one to four carbon atoms in its backbone structure, which may be straight or branched-chain. Examples of lower alkyl groups include methyl, ethyl, n-propyl, i-propyl, tert-butyl, hexyl, heptyl, octyl and so forth. In a preferred embodiment, the term “lower alkyl” includes a straight chain alkyl having 4 or fewer carbon atoms in its backbone, e.g., C₁-C₄ alkyl.

The term “alkoxy,” as used herein, refers to an alkyl or a cycloalkyl group which is linked to another moiety though an oxygen atom. Alkoxy groups can be optionally substituted with one or more substituents.

The terms “alkoxyalkyl,” “polyaminoalkyl” and “thioalkoxyalkyl” refer to alkyl groups, as described above, which further include oxygen, nitrogen or sulfur atoms replacing one or more carbons of the hydrocarbon backbone, e.g., oxygen, nitrogen or sulfur atoms.

The terms “alkenyl” and “alkynyl” refer to unsaturated aliphatic groups analogous in length and possible substitution to the alkyls described above, but that contain at least one double or triple bond, respectively. For example, the invention contemplates cyano and propargyl groups.

The term “ameliorate” means to decrease, suppress, attenuate, diminish, arrest, or stabilize the development or progression of a disease.

The term “alteration” refers to a change (increase or decrease) in a parameter as detected by standard art known methods, such as those described herein.

The term “aryl” refers to the radical of aryl groups, including 5- and 6-membered single-ring aromatic groups that may include from zero to four heteroatoms, for example, benzene, pyrrole, furan, thiophene, imidazole, benzoxazole, benzothiazole, triazole, tetrazole, pyrazole, pyridine, pyrazine, pyridazine and pyrimidine, and the like. Aryl groups also include polycyclic fused aromatic groups such as naphthyl, quinolyl, indolyl, and the like. Those aryl groups having heteroatoms in the ring structure may also be referred to as “aryl heterocycles,” “heteroaryls” or “heteroaromatics.” The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The term “cancer” refers to a malignant tumor of potentially unlimited growth that expands locally by invasion and systemically by metastasis.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “chiral” refers to molecules which have the property of non-superimposability of the mirror image partner, while the term “achiral” refers to molecules which are superimposable on their mirror image partner.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the object to be detected.

By “disease” is meant any condition or disorder that damages or interferes with the normal function of a cell, tissue, or organ.

The term “diastereomers” refers to stereoisomers with two or more centers of dissymmetry and whose molecules are not mirror images of one another.

The term “effective amount” refers to the amount of an agent required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

A therapeutically effective amount of a compound delineated herein (i.e., an effective dosage) may range from about 0.1 μg to 20 milligram per kilogram of body weight per day (mg/kg/day) (e.g., 0.1 μm/kg to 2 mg/kg, 0.3-3 μm/kg, 0.18-0.54 mg/kg). In other embodiments, the amount varies from about 0.1 mg/kg/day to about 100 mg/kg/day. In still other embodiments, the amount varies from about 0.001 μg to about 100 μg/kg (e.g., of body weight). One of skill in the art can readily extrapolate from dosages shown to be effective in in vivo testing to dosages that are likely to be effective in humans. In one embodiment, about 0.1-200 mg/kg/day a compound of the invention (e.g., any of compounds 120, 12, 61, 1, 56, 31, and 196) is administered to a mouse, preferably 1-100 mg/kg, more preferably 5-50 mg/kg. In another embodiment, a dog receives 1-20 mg/kg of such compounds. In another embodiment, a human subject receives 0.1 μm/kg to 2 mg/kg of a compound of the invention (e.g., any of compounds 120, 12, 61, 1, 56, 31, and 196) per day. In yet another embodiment, 0.3-3 μm/kg of such compounds is administered to a human subject. In still another embodiment, 0.18-0.54 mg/kg total per day is administered to a human subject. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a compound delineated herein can include a single treatment or, preferably, can include a series of treatments. In one example, a subject is treated with a compound delineated herein in the range of between about 0.1 μg to 20 milligram per kilogram of body weight per day (mg/kg/day) (e.g., 0.1 μm/kg to 2 mg/kg, 0.3-3 μm/kg, 0.18-0.54 mg/kg). In other embodiments, the amount varies from about 0.1 mg/kg/day to about 100 mg/kg/day. In still other embodiments, the amount varies from about 0.001 μg to about 100 μg/kg (e.g., of body weight). If desired, the dosage is administered one time per day, two times per day, or one time per week. Treatment is carried out for between about 1 to 10 weeks, preferably between 2 to 8 weeks, more preferably between about 3 to 7 weeks, and even more preferably for about 4, 5, or 6 weeks. It will also be appreciated that the effective dosage of a compound delineated herein used for treatment may increase or decrease over the course of a particular treatment.

The term “enantiomers” refers to two stereoisomers of a compound which are non-superimposable mirror images of one another. An equimolar mixture of two enantiomers is called a “racemic mixture” or a “racemate.”

The term “halogen” designates —F, —Cl, —Br or —I.

The term “haloalkyl” is intended to include alkyl groups as defined above that are mono-, di- or polysubstituted by halogen, e.g., fluoromethyl and trifluoromethyl.

The term “hydroxyl” means —OH.

The term “heteroatom” as used herein means an atom of any element other than carbon or hydrogen. Preferred heteroatoms are nitrogen, oxygen, sulfur and phosphorus.

The term “heteroaryl” refers to an aromatic 5-8 membered monocyclic, 8-12 membered bicyclic, or 11-14 membered tricyclic ring system having 1-4 ring heteroatoms if monocyclic, 1-6 heteroatoms if bicyclic, or 1-9 heteroatoms if tricyclic, said heteroatoms selected from O, N, or S, and the remainder ring atoms being carbon. Heteroaryl groups may be optionally substituted with one or more substituents. Examples of heteroaryl groups include, but are not limited to, pyridyl, furanyl, benzodioxolyl, thienyl, pyrrolyl, oxazolyl, oxadiazolyl, imidazolyl thiazolyl, isoxazolyl, quinolinyl, pyrazolyl, isothiazolyl, pyridazinyl, pyrimidinyl, pyrazinyl, triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, and indolyl.

The term “heterocyclic” as used herein, refers to organic compounds that contain at least at least one atom other than carbon (e.g., S, O, N) within a ring structure. The ring structure in these organic compounds can be either aromatic or non-aromatic. Some examples of heterocyclic moeities include, are not limited to, pyridine, pyrimidine, pyrrolidine, furan, tetrahydrofuran, tetrahydrothiophene, and dioxane.

The term “isomers” or “stereoisomers” refers to compounds which have identical chemical constitution, but differ with regard to the arrangement of the atoms or groups in space.

The term “isotopic derivatives” includes derivatives of compounds in which one or more atoms in the compounds are replaced with corresponding isotopes of the atoms. For example, an isotopic derivative of a compound containing a carbon atom (C¹²) would be one in which the carbon atom of the compound is replaced with the C¹³ isotope.

The term “multidrug resistant” refers to a reduced susceptibility to one or more chemotherapeutic agents.

The term “P-glycoprotein polypeptide” refers to a protein having at least about 85% or more amino acid identity to NCBI Accession No. CAA41558 or a fragment thereof that has ABC transporter activity.

The term “MDR1 polynucleotide” refers to a nucleic acid sequence encoding a P-glycoprotein polypeptide.

The term “neoplastic” refers to those cells having the capacity for autonomous growth, i.e., an abnormal state or condition characterized by rapidly proliferating cell growth. A neoplastic disease state may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The language “inhibiting the growth” of the neoplasm includes slowing, interrupting, arresting or stopping its growth and metastases and does not necessarily indicate a total elimination of the neoplastic growth.

The term “modulate” refers to increases or decreases in a parameter in response to exposure to a compound of the invention.

The common medical meaning of the term “neoplasia” refers to “new cell growth” that results as a loss of responsiveness to normal growth controls, e.g. to neoplastic cell growth. A “hyperplasia” refers to cells undergoing an abnormally high rate of growth. However, as used herein, the term neoplasia generally refers to cells experiencing abnormal cell growth rates. Neoplasias include “tumors,” which may be either benign, premalignant or malignant.

The term “obtaining” as in “obtaining compound” is intended to include purchasing, synthesizing or otherwise acquiring the compound.

The term “optical isomers” as used herein includes molecules, also known as chiral molecules, are exact non-superimposable mirror images of one another.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The terms “polycyclyl” or “polycyclic radical” refer to the radical of two or more cyclic rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkyl, alkylaryl, or an aromatic or heteroaromatic moiety.

The term “polymorph” as used herein, refers to solid crystalline forms of a compound of the present invention or complex thereof. Different polymorphs of the same compound can exhibit different physical, chemical and/or spectroscopic properties. Different physical properties include, but are not limited to stability (e.g., to heat or light), compressibility and density (important in formulation and product manufacturing), and dissolution rates (which can affect bioavailability). Differences in stability can result from changes in chemical reactivity (e.g., differential oxidation, such that a dosage form discolors more rapidly when comprised of one polymorph than when comprised of another polymorph) or mechanical characteristics (e.g., tablets crumble on storage as a kinetically favored polymorph converts to thermodynamically more stable polymorph) or both (e.g., tablets of one polymorph are more susceptible to breakdown at high humidity). Different physical properties of polymorphs can affect their processing.

The term “prodrug” includes compounds with moieties which can be metabolized in vivo. Generally, the prodrugs are metabolized in vivo by esterases or by other mechanisms to active drugs. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Preferred prodrug moieties are propionoic acid esters and acyl esters. Prodrugs which are converted to active forms through other mechanisms in vivo are also included.

The language “a prophylactically effective anti-neoplastic amount” of a compound refers to an amount of a compound delineated herein or otherwise described herein which is effective, upon single or multiple dose administration to the patient, in preventing or delaying the occurrence of the onset of a neoplastic disease state.

Furthermore the indication of stereochemistry across a carbon-carbon double bond is also opposite from the general chemical field in that “Z” refers to what is often referred to as a “cis” (same side) conformation whereas “E” refers to what is often referred to as a “trans” (opposite side) conformation. Both configurations, cis/trans and/or Z/E are encompassed by the compounds of the present invention.

With respect to the nomenclature of a chiral center, the terms “d” and “1” configuration are as defined by the IUPAC Recommendations. As to the use of the terms, diastereomer, racemate, epimer and enantiomer, these will be used in their normal context to describe the stereochemistry of preparations.

By “reference” is meant a standard or control condition.

The term “subject” includes organisms which are capable of suffering from a neoplasia or who could otherwise benefit from the administration of a compound of the invention, such as human and non-human animals. Preferred human animals include human patients suffering from or prone to suffering from a neoplasia, as described herein. The term “non-human animals” of the invention includes all vertebrates, e.g., mammals, e.g., rodents, e.g., mice, and non-mammals, such as non-human primates, also sheep, dog, cow, chickens, amphibians, and reptiles.

The term “sulfhydryl” or “thiol” means —SH.

The phrases “systemic administration,” “administered systemically”, “peripheral administration” and “administered peripherally” as used herein mean the administration of a compound(s), drug or other material, such that it enters the patient's system and, thus, is subject to metabolism and other like processes, for example, subcutaneous administration.

As used herein, the term “tautomers” refers to isomers of organic molecules that readily interconvert by tautomerization, in which a hydrogen atom or proton migrates in the reaction, accompanied in some occasions by a switch of a single bond and an adjacent double bond.

The invention provides a number of targets that are useful for the development of highly specific drugs to treat or prevent a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

### Na⁺/K⁺-ATPase, or Na⁺/K⁺ Pump

The Na⁺/K⁺-ATPase, or Na⁺/K⁺ pump is a complex of integral membrane proteins that actively transports sodium and potassium ions across the cell plasma membrane. In addition to pumping ions across the membrane, the enzyme functions as a receptor for cardiac glycosides, such as ouabain, digoxin, marinobufagenin and others (reviewed in Mijatovic T et al., Biochim Biophys Acta. 2007; 1776:32-57). Surprisingly, compounds of the invention were discovered to have Na⁺/K⁺-ATPase inhibitory activity.

Compounds of the invention are chemically unrelated to cardiac glycosides, which may also display Na⁺/K⁺-ATPase inhibitory activity. Cardiac glycosides comprise steroidal and glycoside moieties, and have a lactone ring at the C17 position. In contrast, compounds of the invention have no lactone ring at C17. Despite these distinguishing structural features, Na⁺/K⁺-ATPase, or Na⁺/K⁺ pump effectively inhibited Na⁺/K⁺-ATPase.

### Compounds of the Invention

In one aspect, the invention provides a compound of Formula (I):

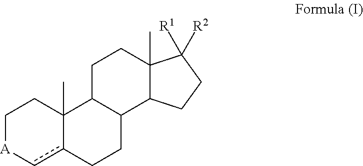

wherein:

“ [symbol] ” represents a single bond or double bond in the ring structure of Formula (I);

One of R¹ and R² is H, and the other is —OR³; alkyl optionally substituted with hydroxyl or alkoxy; —C(O)—NH₂; —C(O)—O-alkyl; —NHR⁸; alkynyl; or R¹ and R² together with the carbon to which they are attached form C═O, C═C(R⁹)₂, or C═N—OR¹⁰;

R³ is H; —NO₂; carbonylalkyl; carbonylaryl; or aminoalkyl optionally substituted with one or more alkyl;

A is CH(OR⁴), C═N—OR⁵, CH—CH═N—OR⁶, C═CH—CH═N—OR⁶, CH—CH═CH—R⁶, CH—SR⁷, or CH—S(O)R⁷;

R⁴ is alkyl optionally substituted by a 4- to 6-membered heterocyclic ring, a 5 to 14-membered heteroaryl, amino(C₁₋C₄)alkoxyl, (C₁₋C₄)alkoxyl substituted by a 4- or 6-membered heterocyclic ring, or guanidinyl; aminoalkyl optionally substituted by one or more alkyl; or aminoacyl optionally substituted by alkyl; wherein said heterocyclic or heteroaryl ring is further optionally substituted by one or more alkyl

R⁵ is aminoalkyl optionally substituted by one or more alkyl or acetyl; alkyl optionally substituted by guanidinyl, heteroaryl that is further optionally substituted by one or more alkyl or aminoalkyl optionally substituted by one or more alkyl, or a 4- to 6-membered heterocyclic ring that is further optionally substituted by one or more alkyl; or a 4- to 6-membered heterocyclic ring optionally substituted by one or more alkyl;

R⁶ and R⁷, each independently, are aminoalkyl optionally substituted by one or more alkyl;

R⁸ is H or formyl;

R⁹, for each occurrence, is the same or different and is H or halogen;

R¹⁰ is H or alkyl; and

tautomers, stereoisomers, Z and E isomers, optical isomers, N-oxides, hydrates, polymorphs, pharmaceutically acceptable esters, salts, prodrugs and/or isotopic derivatives thereof.

One embodiment provides that the “ [symbol] ” in Formula (I) represents a single bond in the ring structure. A separate embodiment provides that the “ [symbol] ” in Formula (I) represents a double bond.

In one embodiment, R¹ and R² together with the carbon atom to which they are attached form C═O in Formula (I).

In one embodiment, A is C═N—OR⁵.

One embodiment provides that R⁵ is amino(C₁-C₄)alkyl optionally substituted by one or more (C₁-C₄)alkyl or acetyl groups. R⁵ can be, but is not limited to, aminoethyl, 2-aminopropyl, 2-methyl-2-aminopropyl, 2-aminomethyl-2-propyl, N-methylamino-ethyl, aminopropyl, N-methylamino-propyl, aminobutyl, N-ethylamino-butyl, N,N-dimethylamino-ethyl, or N-acetylamino-ethyl.

In another embodiment, R⁵ is (C₁-C₄)alkyl optionally substituted by guanidinyl, a 5- to 14-membered heteroaryl or a 4- to 6-membered heterocyclic ring, wherein 5- to 14-membered heteroaryl is optionally substituted by one or more (C₁-C₄)alkyl or amino(C₁-C₄)alkyl that is further optionally substituted by one or more (C₁-C₄)alkyl, and said 4- to 6-membered heterocyclic ring is optionally substituted by one or more (C₁-C₄)alkyl.

One particular embodiment provides that R⁵ is (C₁-C₄)alkyl substituted by guanidinyl. In another embodiment, R⁵ is (C₁-C₄)alkyl substituted by a 5- to 14-membered heteroaryl that is optionally substituted by one or more (C₁-C₄)alkyl or amino(C₁-C₄)alkyl, wherein said amino(C₁-C₄)alkyl is further optionally substituted by one or more (C₁-C₄)alkyl. A separate embodiment provides that R⁵ is (C₁-C₄)alkyl that is substituted by a 4- to 6-membered heterocyclic ring, wherein said 4- to 6-membered heterocyclic ring is optionally substituted by one or more (C₁-C₄)alkyl. One example of said 4- to 6-membered heterocyclic ring is a pyrrolidinyl ring.

Yet another embodiment provides that R⁵ is a 4- to 6-membered heterocyclic ring that is optionally substituted by one or more (C₁-C₄)alkyl. For example, R⁵ can be a pyrrolidinyl, azetidinyl, or piperidinyl ring, which is optionally substituted by one or more (C₁-C₄)alkyl.

One embodiment provides that R⁵ is (C₁-C₄)alkyl substituted by a 5- or 6-membered heteroaryl that is optionally substituted by one or more (C₁-C₄)alkyl. A separate embodiment provides that R⁵ is (C₁-C₄)alkyl substituted by a 5- or 6-membered heteroaryl that is substituted by amino(C₁-C₄)alkyl, and said amino(C₁-C₄)alkyl is optionally substituted by one or more (C₁-C₄)alkyl.

Certain examples of the 5- or 6-membered heteroaryl group include an oxazolyl, isoxazolyl and oxadiazolyl ring, wherein said oxazolyl, isoxazolyl or oxadiazolyl ring is further optionally substituted by one or more (C₁-C₄)alkyl. Other examples of the 5- or 6-membered heteroaryl group include an oxazolyl, isoxazolyl and oxadiazolyl ring, wherein said oxazolyl, isoxazolyl or oxadiazolyl ring is substituted by amino(C₁-C₄)alkyl, wherein said amino(C₁-C₄)alkyl is further optionally substituted by one or more (C₁-C₄)alkyl.

The 5- or 6-membered heteroaryl group may also be a triazolyl ring, wherein said triazolyl ring is substituted by amino(C₁-C₄)alkyl that is further optionally substituted by one or more (C₁-C₄)alkyl.

In one embodiment, R⁴ is (C₁-C₄)alkyl that is optionally substituted by a 4- to 6-membered heterocyclic ring, a 5 to 14-membered heteroaryl, amino(C₁₋C₄)alkoxyl, (C₁₋C₄)alkoxyl substituted by a 4- to 6-membered heterocyclic ring, or guanidinyl.

One embodiment provides that R⁴ is (C₁-C₄)alkyl substituted by a 4- to 6-membered heterocyclic ring, or a 5- or 6-membered heteroaryl, wherein said heterocyclic or heteroaryl ring is optionally substituted by one or more (C₁-C₄)alkyl. For example, R⁴ can be (C₁-C₄)alkyl substituted by a pyrrolidinyl, azetidinyl, 4-methylpiperazinyl, piperazinyl, imidazolyl, or piperidinyl ring.

In another embodiment, A is CH(OR⁴).

Another embodiment provides that R⁴ is (C₁-C₄)alkyl optionally substituted by amino(C₁-C₄)alkoxyl, or (C₁-C₄)alkoxyl substituted by a 4- to 6-membered heterocyclic ring. For example, R⁴ may be (C₁-C₄)alkyl substituted by N,N-dimethylamino-1-ethoxyl or pyrrolidinyl-1-ethoxyl.

Another embodiment provides that R⁴ is (C₁-C₄)alkyl that is substituted by guanidinyl.

A separate embodiment provides that R⁴ is amino(C₁-C₄)alkyl optionally substituted by one or more (C₁-C₄)alkyl. Examples of the R⁴ group include aminoethyl, N-methylaminoethyl, N,N-dimethylaminoethyl, 3-aminopropyl, N-methylamino-1-propyl, N,N-dimethylamino-1-propyl, and N,N-dimethylamino-1-butyl.

Yet another embodiment provides that R⁴ is amino(C₁-C₄)acyl optionally substituted by one or more (C₁-C₄)alkyl. In certain instances, R⁴ is 3-aminopropanoyl, 3-aminobutanoyl, or aminomethylpropanoyl.

In a separate embodiment, A is CH—CH═N—OR⁶. One embodiment provides that R⁶ is amino(C₁-C₄)alkyl optionally substituted by one or more (C₁-C₄)alkyl.

Yet another embodiment provides that A is C═CH—CH═N—OR⁶.

Embodiments of the invention also provide that A is CH—CH═CH—R⁶. R⁶ may be amino(C₁-C₄)alkyl optionally substituted by one or more (C₁-C₄)alkyl. For instance, R⁶ is 3-aminopropyl or aminoethyl.

Other embodiments of the invention provide that A is CH—SR⁷ or CH—S(O)R⁷. R⁷ may be, but is not limited to, 3-aminopropyl.

A separate embodiment provides a compound of Formula (I), wherein one of R¹ and R² is H, and the other is —OR³. One embodiment provides that one of R¹ and R² is H, and the other is —OH. Another embodiment provides that R³ is —NO₂. A separate embodiment provides that R³ is carbonyl(C₁-C₄)alkyl. Yet R³ may also be —C(O)-aryl, wherein said aryl is a 5- to 14-membered aryl group. For example, R³ is benzoyl. In certain embodiments, R³ may be amino(C₁-C₄)alkyl optionally substituted with one or more (C₁-C₄)alkyl.

In one embodiment, A is C═N—OR⁵. In another embodiment, A is CH(OR⁴). Yet another embodiment provides that A is CH—CH═CH—R⁶. Separate embodiments provide that A is CH—CH═N—OR⁶ or C═CH—CH═N—OR⁶. Yet one embodiment provides that A is CH—SR⁷. Another embodiment provides that A is CH—S(O)R⁷.

One embodiment provides that R⁵ is amino(C₁-C₄)alkyl optionally substituted by one or more (C₁-C₄)alkyl. R⁵ may be, but is not limited to, aminoethyl, 2-aminopropyl, 2-methyl-2-aminopropyl, 2-aminomethyl-2-propyl, N-methylamino-ethyl, 3-aminopropyl, N-methylamino-propyl, aminobutyl, N-ethylamino-butyl, N,N-dimethylamino-ethyl, or N-acetylamino-ethyl. In certain instances, R⁵ is N,N-dimethylamino-ethyl, aminoethyl, or 3-aminopropyl.

In another embodiment, R⁵ is (C₁-C₄)alkyl optionally substituted by guanidinyl, a 5- to 14-membered heteroaryl, or a 4- to 6-membered heterocyclic ring, wherein said heteroaryl is optionally substituted by one or more (C₁-C₄)alkyl or amino(C₁-C₄)alkyl, and said heterocyclic ring is optionally substituted by one or more (C₁-C₄)alkyl.

One embodiment provides that R⁵ is (C₁-C₄)alkyl that is substituted by a 5- or 6-membered heteroaryl, wherein said heteroaryl is substituted by amino(C₁-C₄)alkyl that is optionally substituted by one or more (C₁-C₄)alkyl. In one instance, R⁵ is (C₁-C₄)alkyl substituted by a triazolyl ring, wherein said triazolyl ring is optionally substituted by aminomethyl, aminoethyl, N,N-dimethylaminomethyl, or N,N-dimethylaminoethyl. In one instance, R⁵ is (C₁-C₄)alkyl substituted by an isoxazolyl or oxazolyl ring, which is substituted by (C₁-C₄)alkyl or amino(C₁-C₄)alkyl. Another instance provides that R⁵ is (C₁-C₄)alkyl substituted by an oxadiazolyl ring, which is substituted by (C₁-C₄)alkyl or amino(C₁-C₄)alkyl.

Certain embodiments provide that R⁴ is (C₁-C₄)alkyl optionally substituted by a 4- to 6-membered heterocyclic ring, a 5 to 14-membered heteroaryl, amino(C₁₋C₄)alkoxyl, (C₁₋C₄)alkoxyl substituted by a 4- or 6-membered heterocyclic ring, or guanidinyl; wherein said heterocyclic or heteroaryl ring is further optionally substituted by one or more (C₁-C₄)alkyl. One instance provides that R⁴ is (C₁-C₄)alkyl substituted by guanidinyl. Another instance provides that R⁴ is (C₁-C₄)alkyl substituted by a 4- to 6-membered heterocyclic ring. Yet another instance provides that R⁴ is (C₁-C₄)alkyl substituted by amino(C₁₋C₄)alkoxyl, which is optionally substituted by one or more (C₁-C₄)alkyl. Further, R⁴ may also be (C₁-C₄)alkyl substituted by (C₁-C₄)alkoxyl, which is further substituted by a 4- to 6-membered ring. A separate instance provides that R⁴ is amino(C₁-C₄)alkyl optionally substituted by one or more (C₁-C₄)alkyl. Certain examples of R⁴ include N,N-dimethylamino-ethyl, N,N-dimethylamino-propyl, and N,N-dimethylamino-butyl.

In certain embodiments, R⁶ is amino(C₁-C₄)alkyl optionally substituted by one or more (C₁-C₄)alkyl. Examples of R⁶ include aminopropyl and aminoethyl.

In some instances, R⁷ is amino(C₁-C₄)alkyl.

One instance provides a compound of Formula (I), wherein one of R¹ and R² is H, and the other is —OR³, R³ is benzoyl, and A is CH(OR⁴). In a certain embodiment, R⁴ is amino(C₁-C₄)alkyl optionally substituted by one or more (C₁-C₄)alkyl.

Compounds of Formula (I), in accordance with the invention, include those described in Table 1 as follows:

TABLE 1

| | |
|---|---|
| 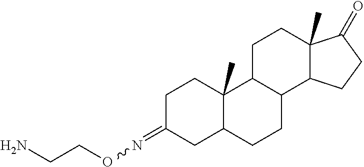 | 1 |
| 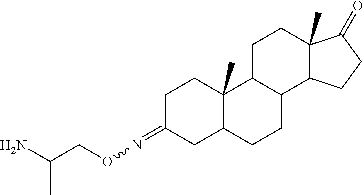 | 2 |
| 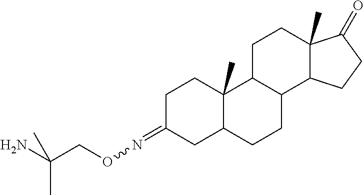 | 3 |
| 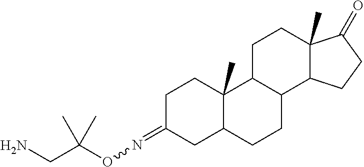 | 4 |
| 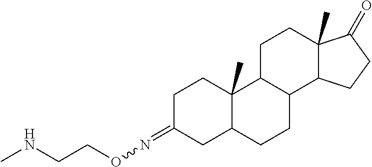 | 5 |
| 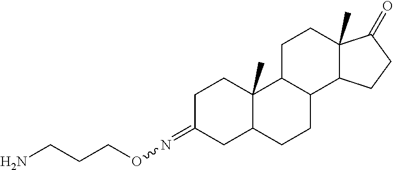 | 6 |
| 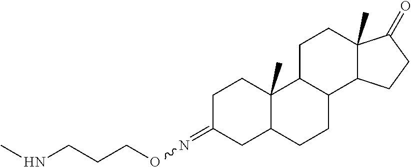 | 7 |
| 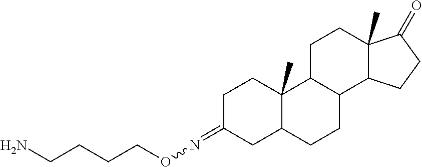 | 8 |
| 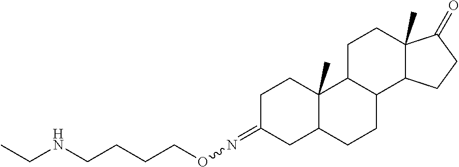 | 9 |
| 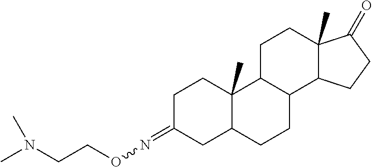 | 10 |
| 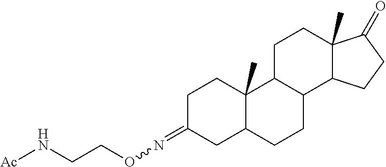 | 11 |
| 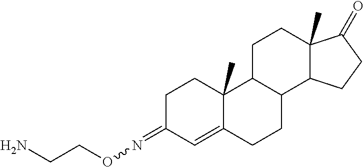 | 12 |
| 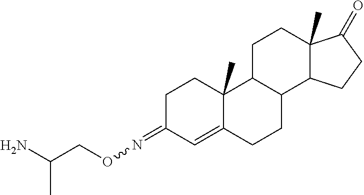 | 13 |
| 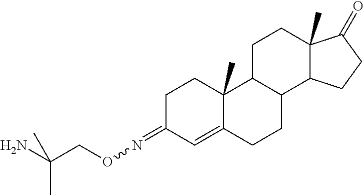 | 14 |
| 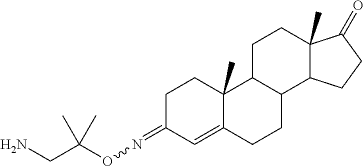 | 15 |
| 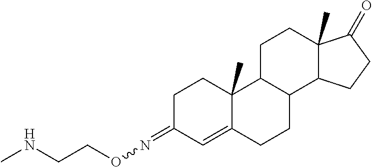 | 16 |
| 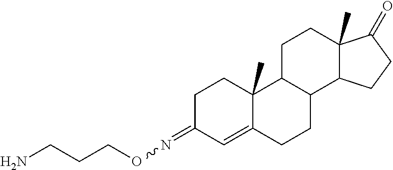 | 17 |
| 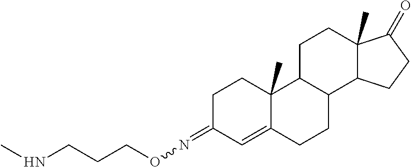 | 18 |
| 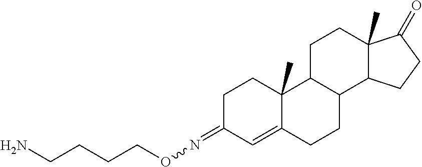 | 19 |
| 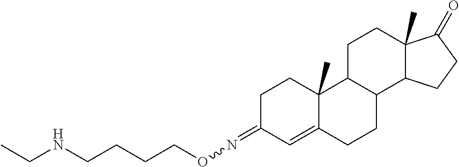 | 20 |
| 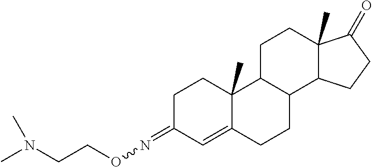 | 21 |
| 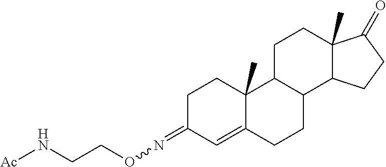 | 22 |
| 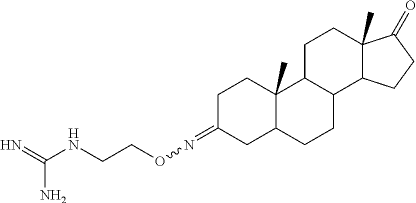 | 23 |
| 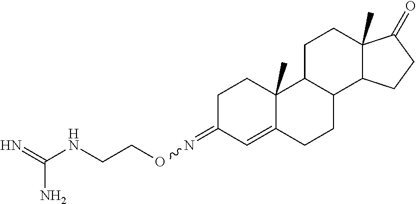 | 24 |
| 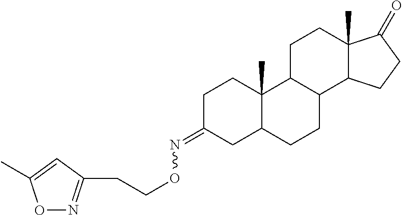 | 25 |
| 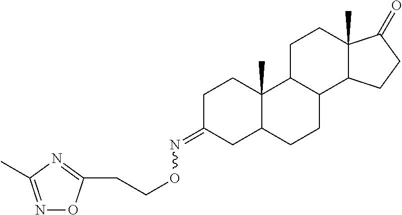 | 26 |
| 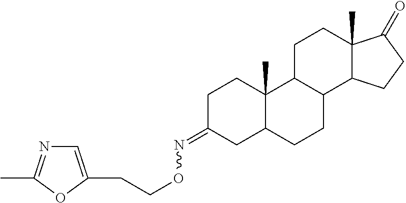 | 27 |
| 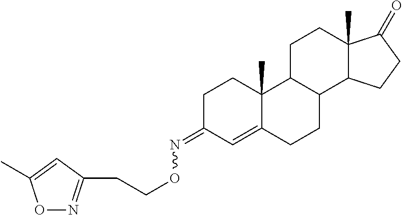 | 28 |
| 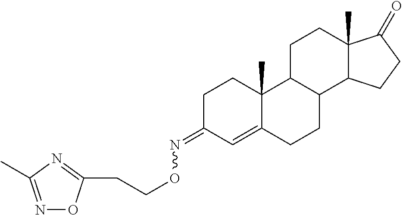 | 29 |
| 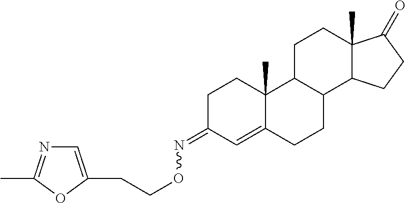 | 30 |
| 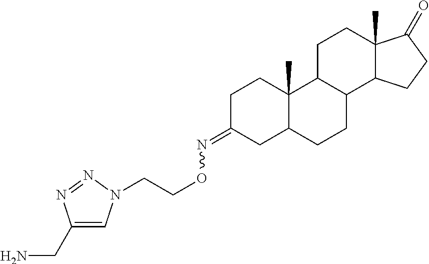 | 31 |
| 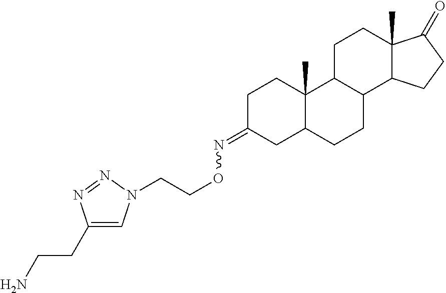 | 32 |
| 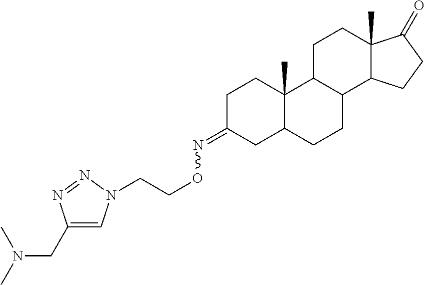 | 33 |
| 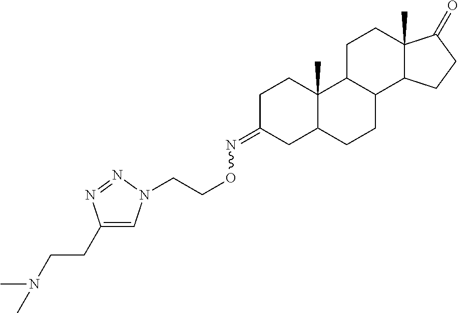 | 34 |
| 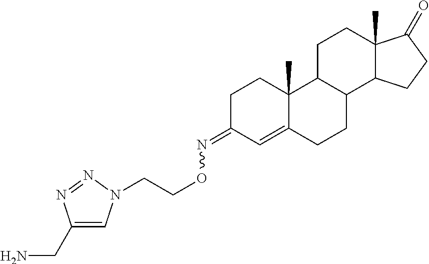 | 35 |
| 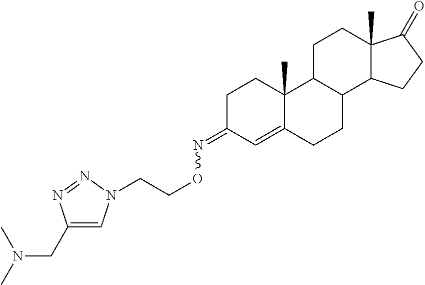 | 36 |
| 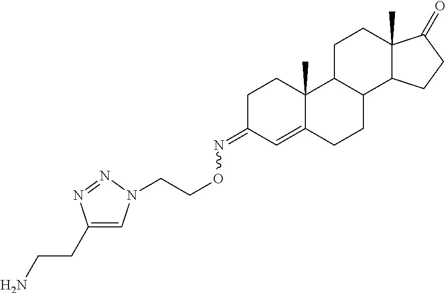 | 37 |
| 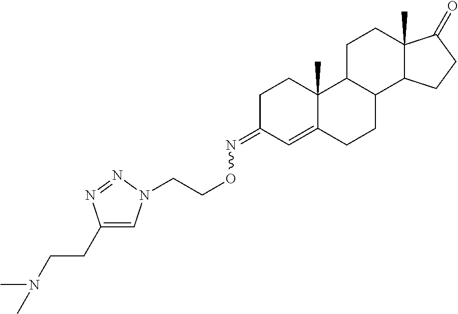 | 38 |
| 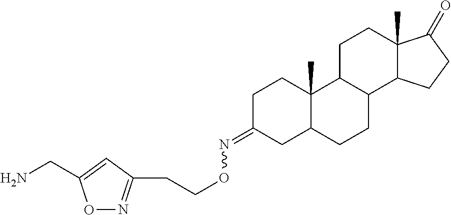 | 39 |
| 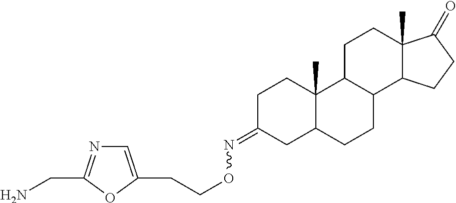 | 40 |
| 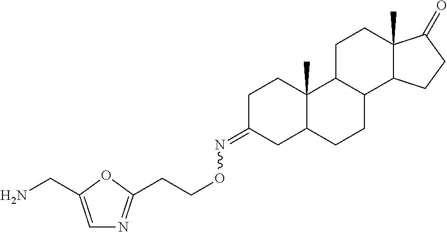 | 41 |
| 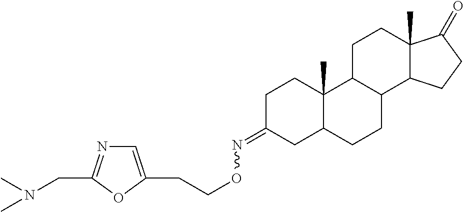 | 42 |
| 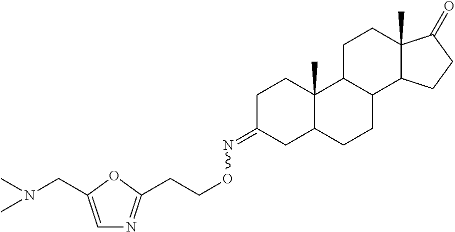 | 43 |
| 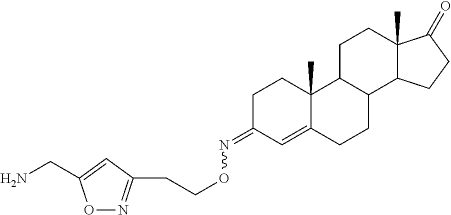 | 44 |
| 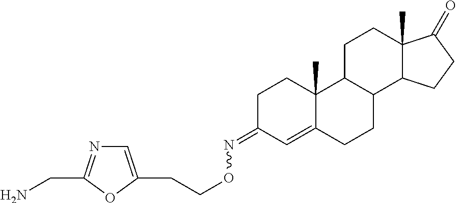 | 45 |
| 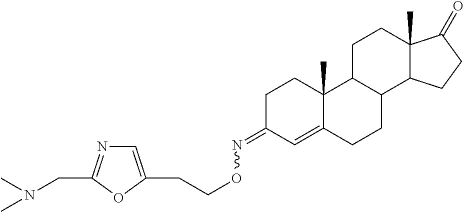 | 46 |
| 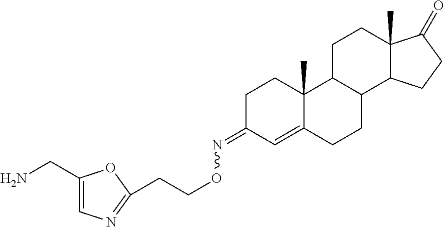 | 47 |
| 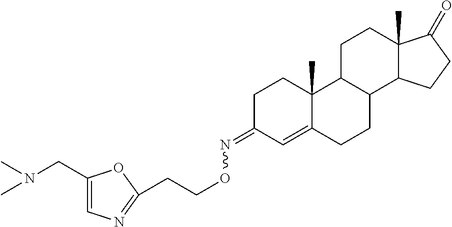 | 48 |
| 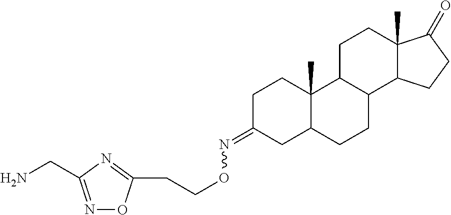 | 49 |
| 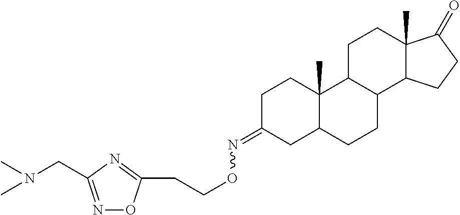 | 50 |
| 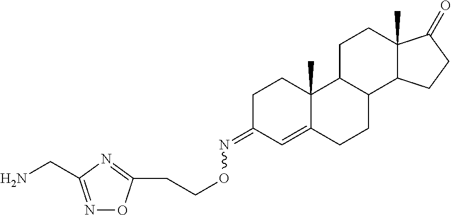 | 51 |
| 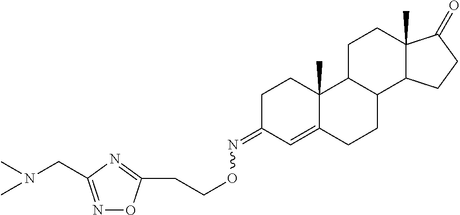 | 52 |
| 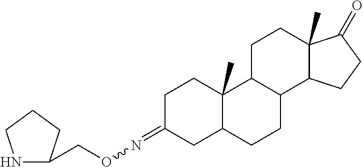 | 53 |
| 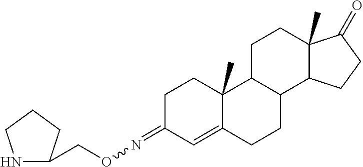 | 54 |
| 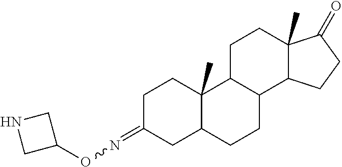 | 55 |
| 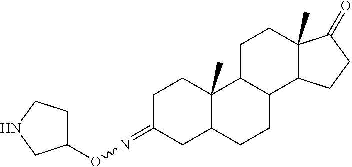 | 56 |
| 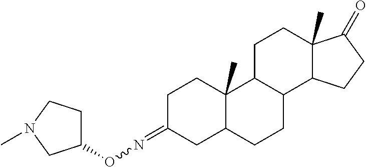 | 57 |
| 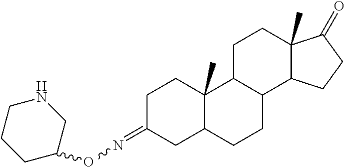 | 58 |
| 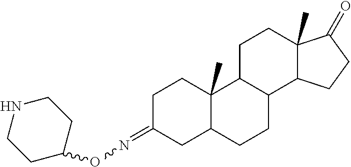 | 59 |
| 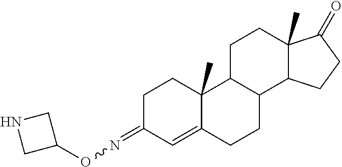 | 60 |
| 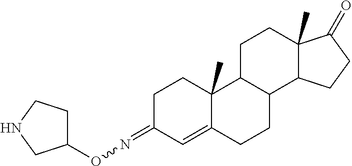 | 61 |
| 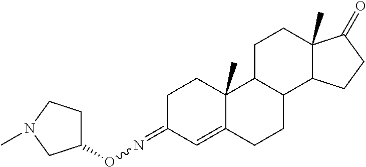 | 62 |
| 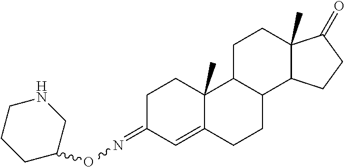 | 63 |
| 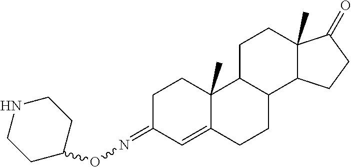 | 64 |
| 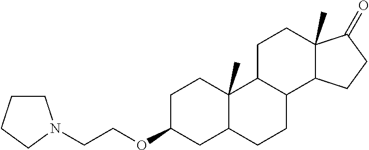 | 65 |
| 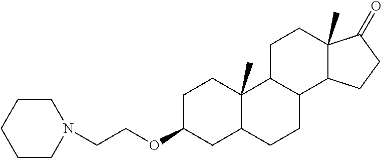 | 66 |
| 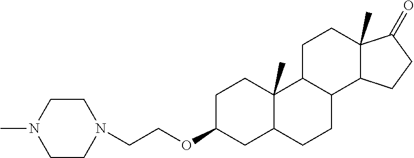 | 67 |
| 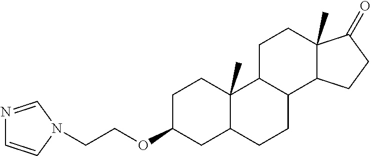 | 68 |
| 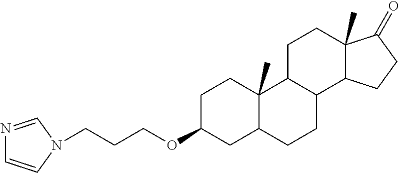 | 69 |
| 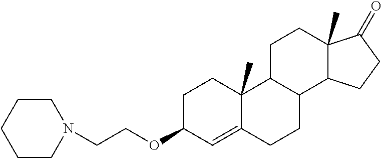 | 70 |
| 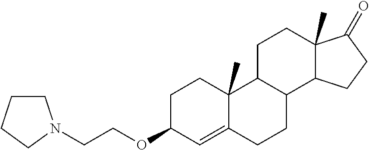 | 71 |
| 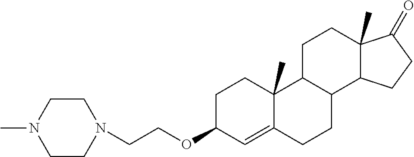 | 72 |
| 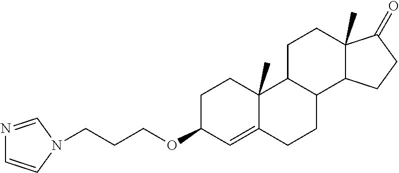 | 73 |
| 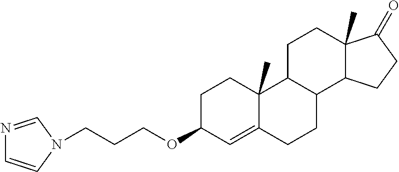 | 74 |
| 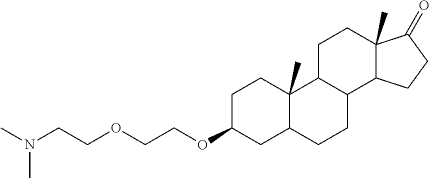 | 75 |
| 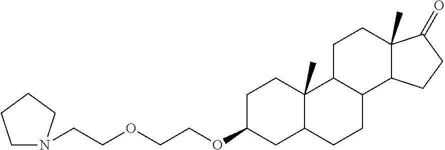 | 76 |
| 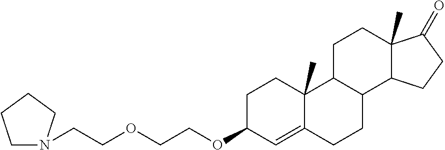 | 77 |
| 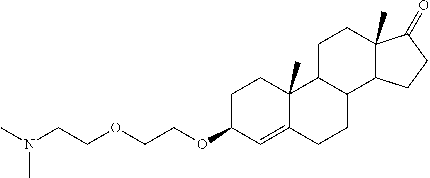 | 78 |
| 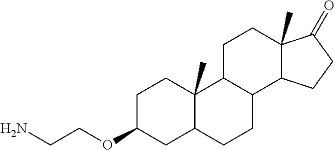 | 79 |
| 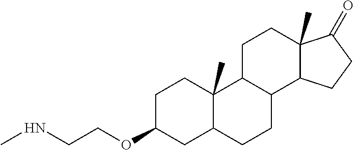 | 80 |
| 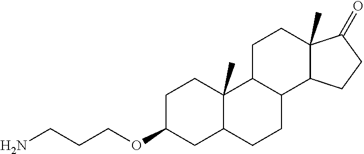 | 81 |
| 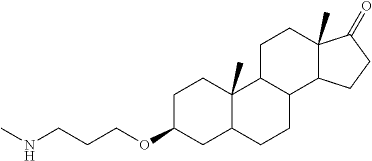 | 82 |
| 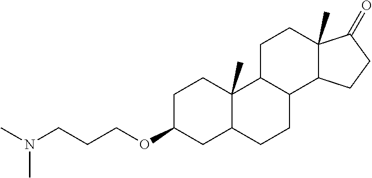 | 83 |
| 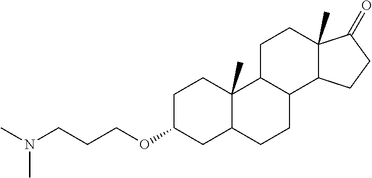 | 84 |
| 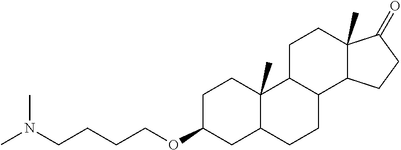 | 85 |
| 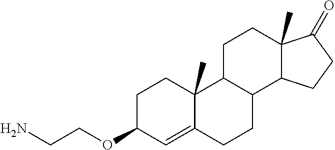 | 86 |
| 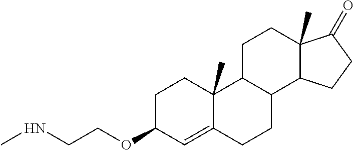 | 87 |
| 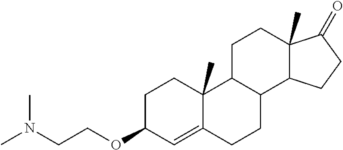 | 88 |
| 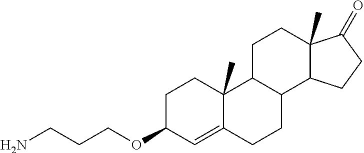 | 89 |
| 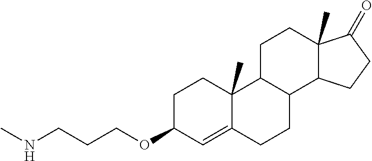 | 90 |
| 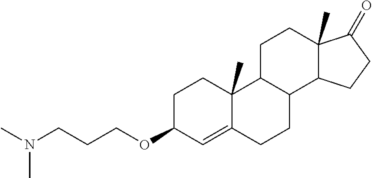 | 91 |
| 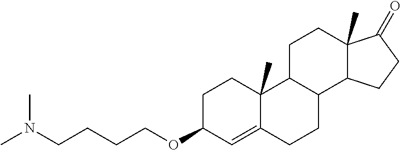 | 92 |
| 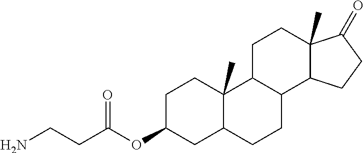 | 93 |
| 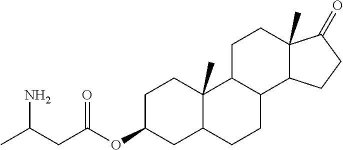 | 94 |
| 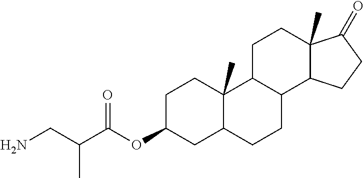 | 95 |
| 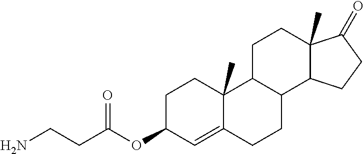 | 96 |
| 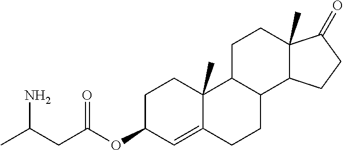 | 97 |
| 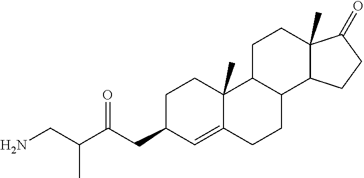 | 98 |
| 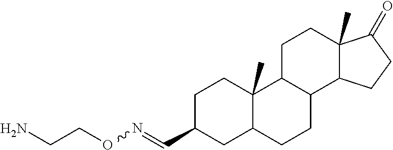 | 99 |
| 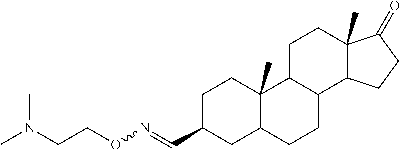 | 100 |
| 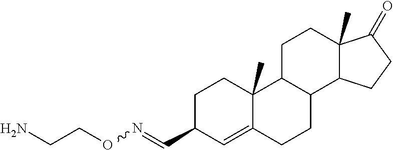 | 101 |
| 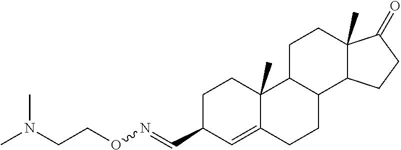 | 102 |
| 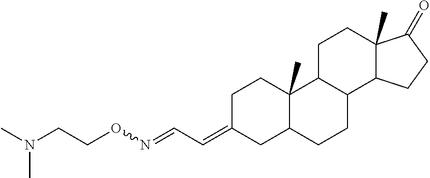 | 103 |
| 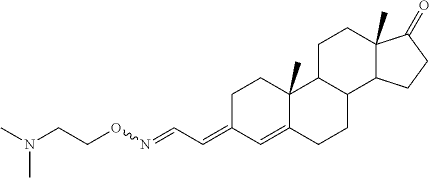 | 104 |
| 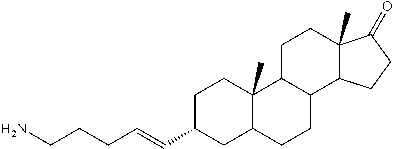 | 105 |
| 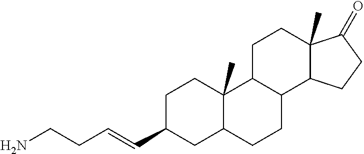 | 106 |
| 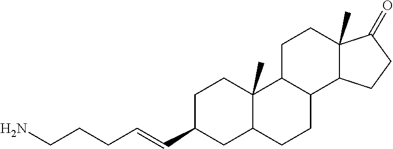 | 107 |
| 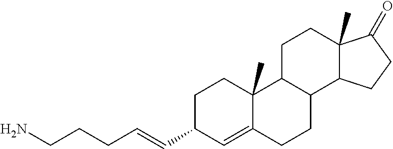 | 108 |
| 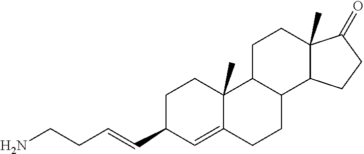 | 109 |
| 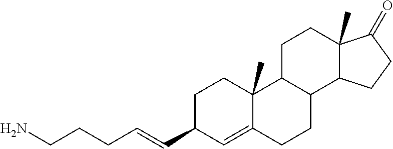 | 110 |
| 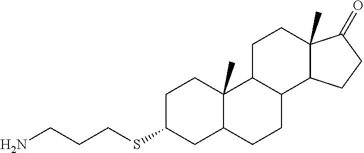 | 111 |
| 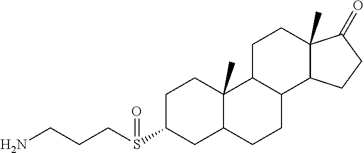 | 112 |
| 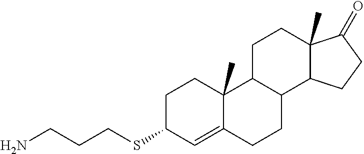 | 113 |
| 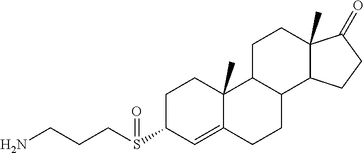 | 114 |
| 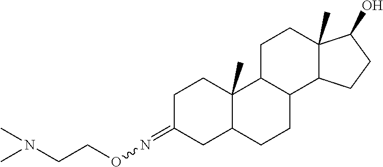 | 115 |
| 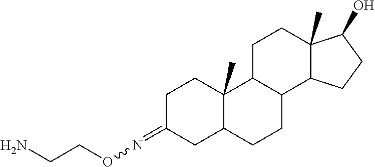 | 116 |
| 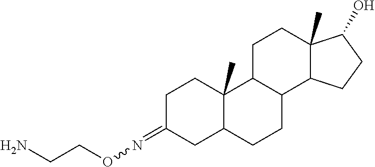 | 117 |
| 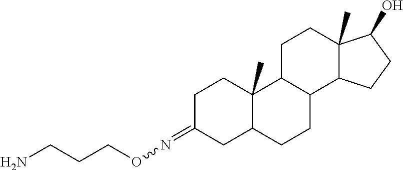 | 118 |
| 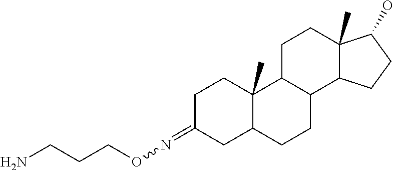 | 119 |
| 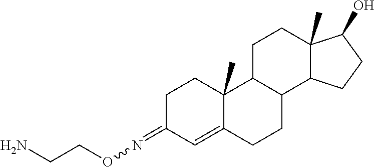 | 120 |
| 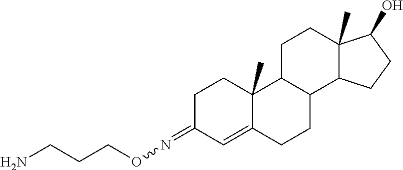 | 121 |
| 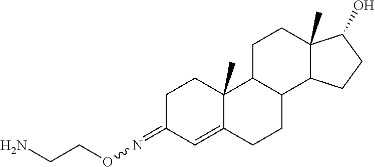 | 122 |
| 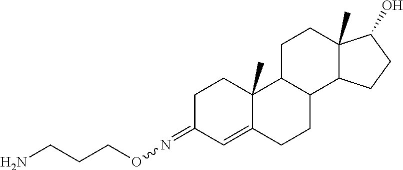 | 123 |
| 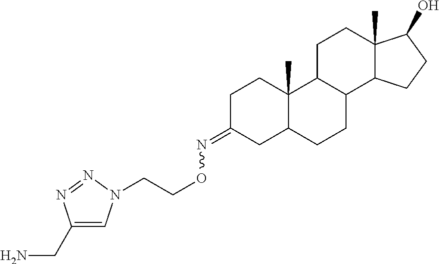 | 124 |
| 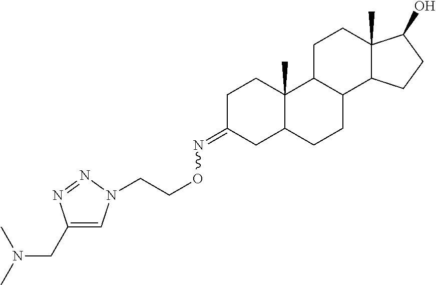 | 125 |
| 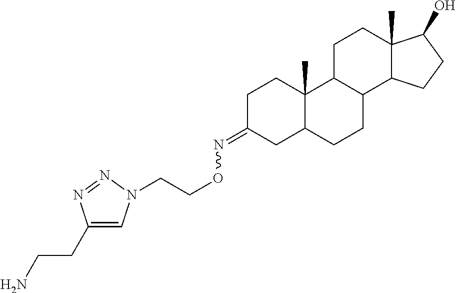 | 126 |
| 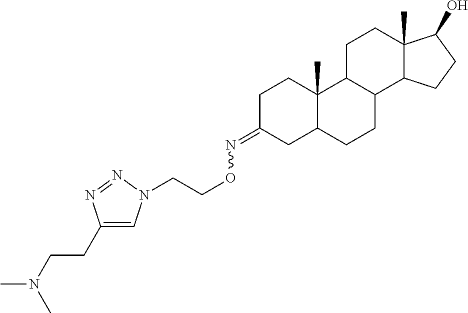 | 127 |
| 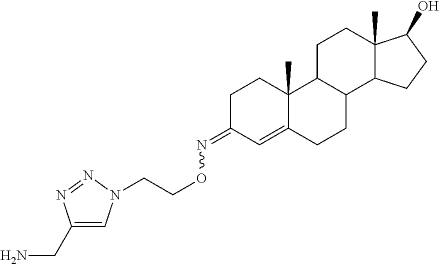 | 128 |
| 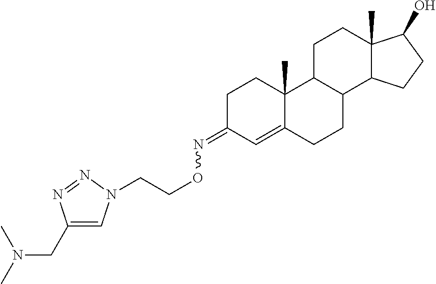 | 129 |
| 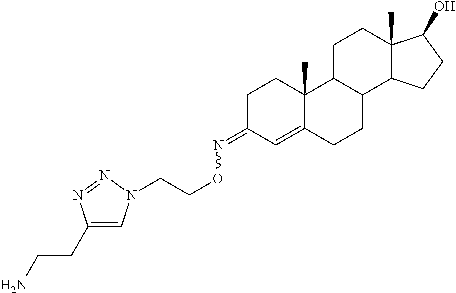 | 130 |
| 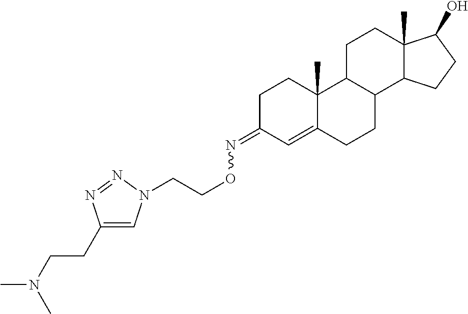 | 131 |
| 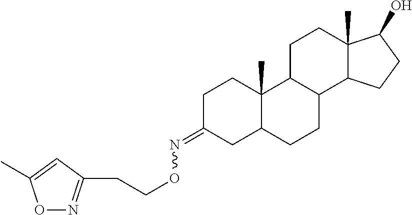 | 132 |
| 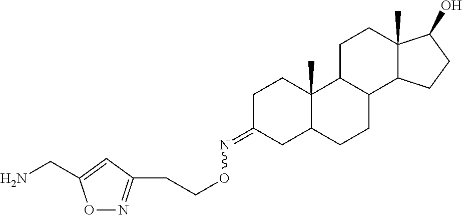 | 133 |
| 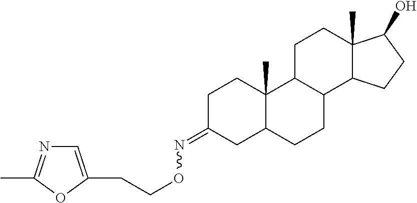 | 134 |
| 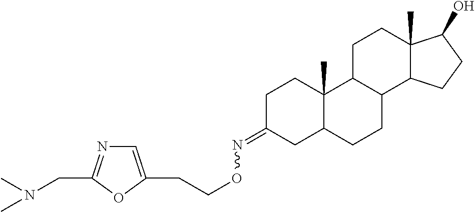 | 135 |
| 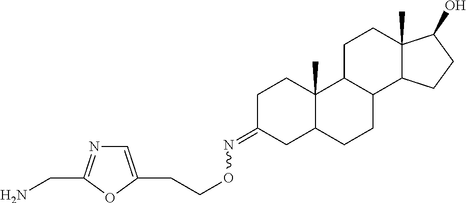 | 136 |
| 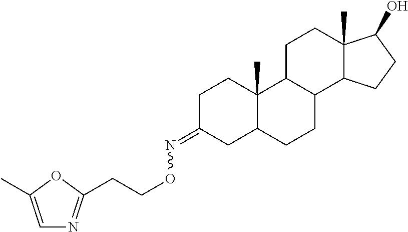 | 137 |
| 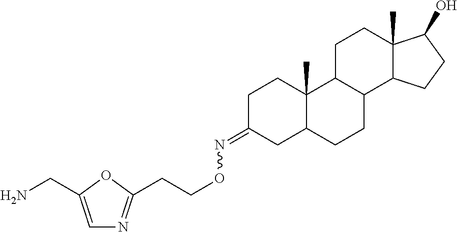 | 138 |
| 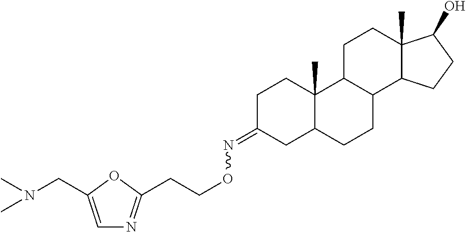 | 139 |
| 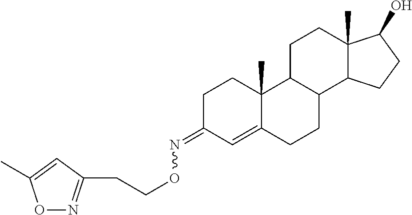 | 140 |
| 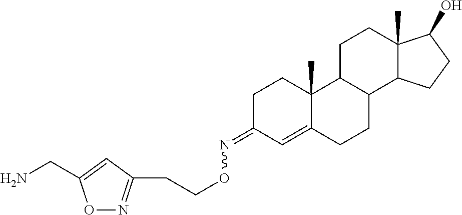 | 141 |
| 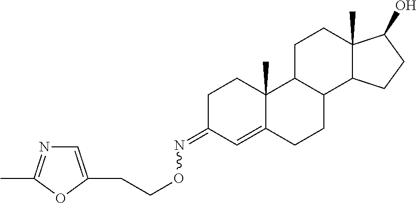 | 142 |
| 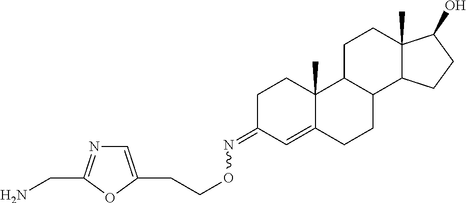 | 143 |
| 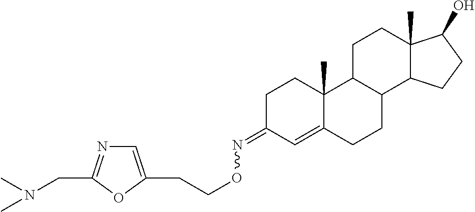 | 144 |
| 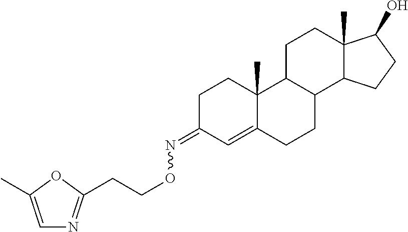 | 145 |
| 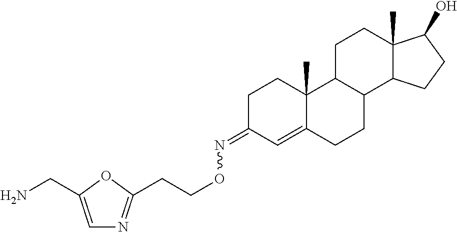 | 146 |
| 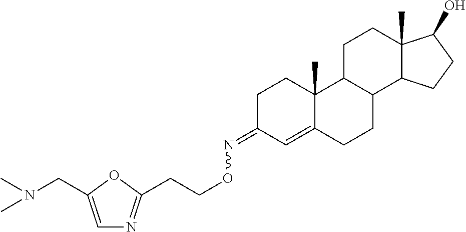 | 147 |
| 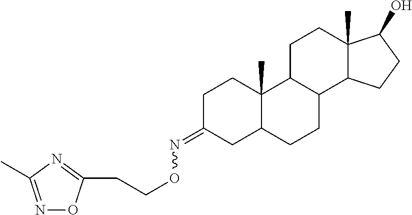 | 148 |
| 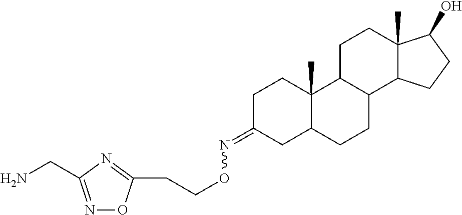 | 149 |
| 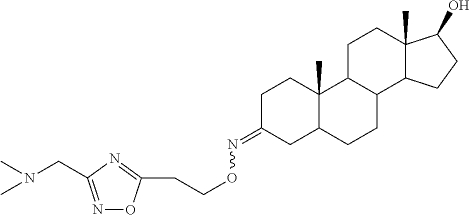 | 150 |
| 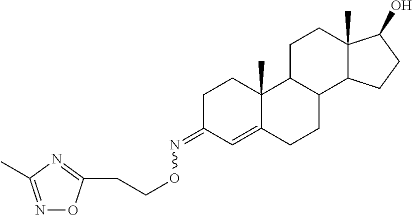 | 151 |
| 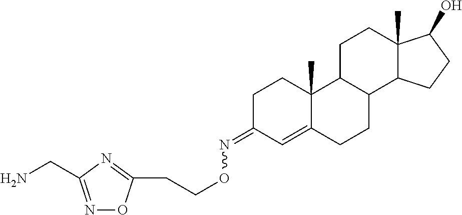 | 152 |
| 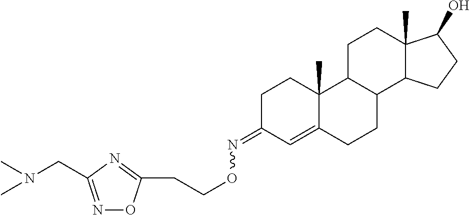 | 153 |
| 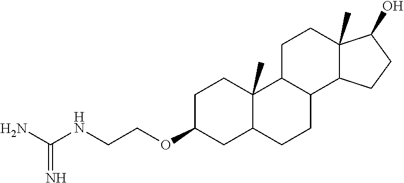 | 154 |
| 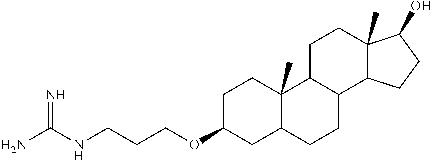 | 155 |
| 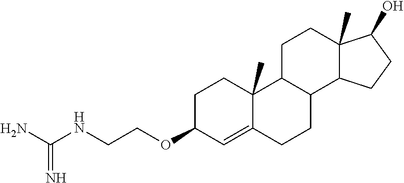 | 156 |
| 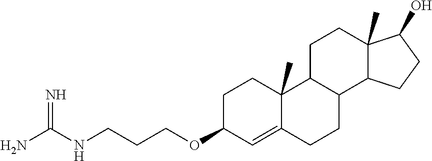 | 157 |
| 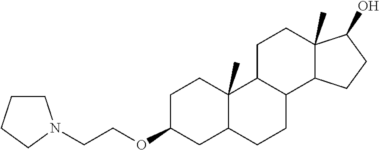 | 158 |
| 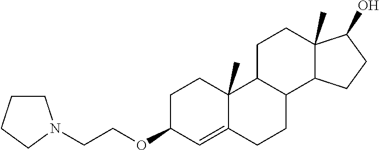 | 159 |
| 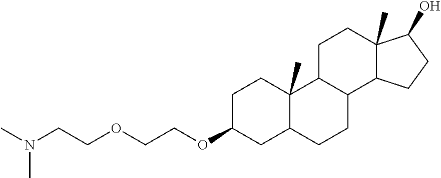 | 160 |
| 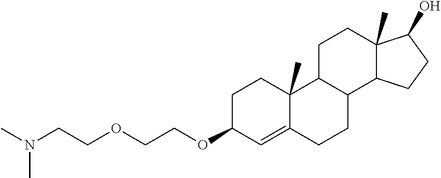 | 161 |
| 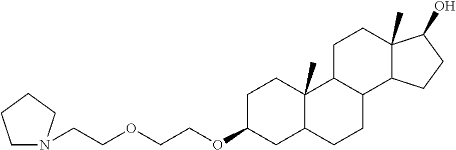 | 162 |
| 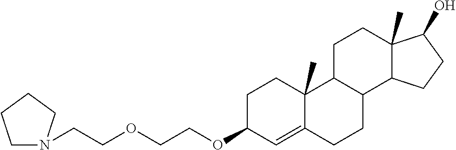 | 163 |
| 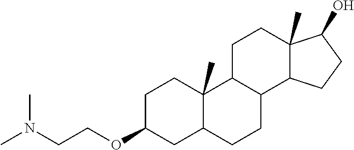 | 164 |
| 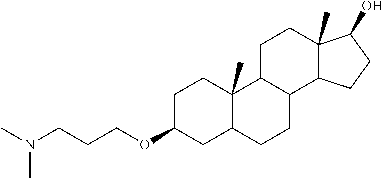 | 165 |
| 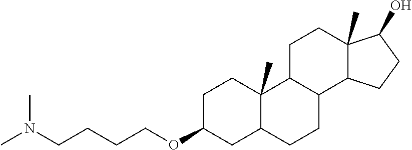 | 166 |
| 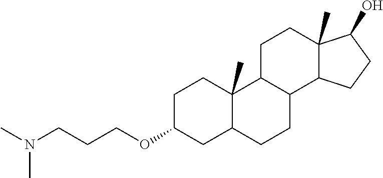 | 167 |
| 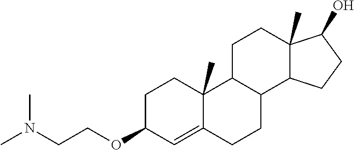 | 168 |
| 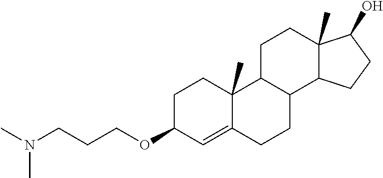 | 169 |
| 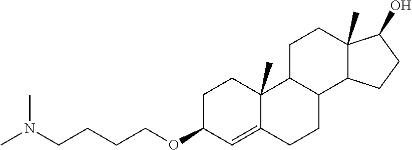 | 170 |
| 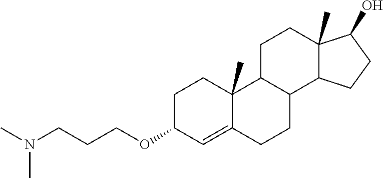 | 171 |
| 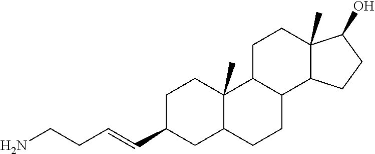 | 172 |
| 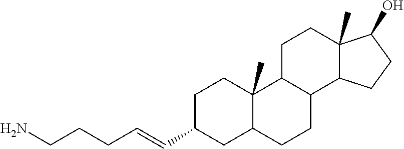 | 173 |
| 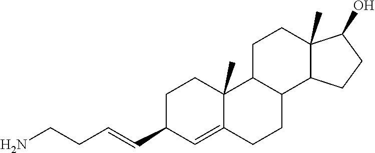 | 174 |
| 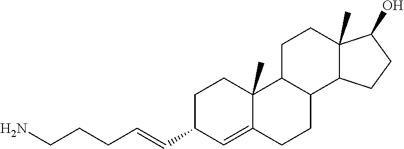 | 175 |
| 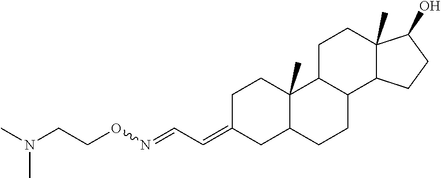 | 176 |
| 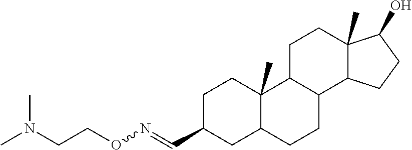 | 177 |
| 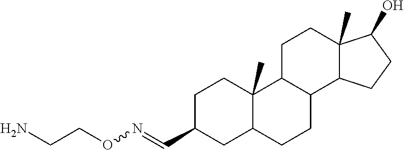 | 178 |
| 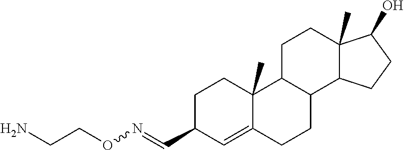 | 179 |
| 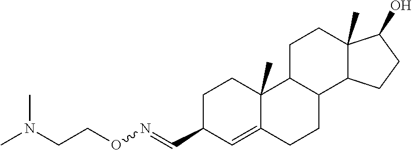 | 180 |
| 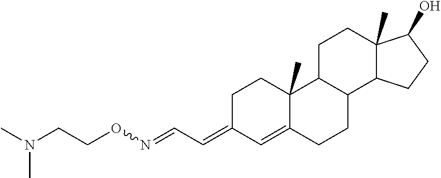 | 181 |
| 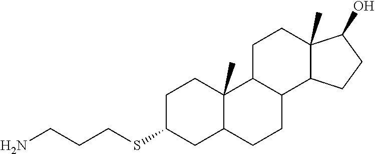 | 182 |
| 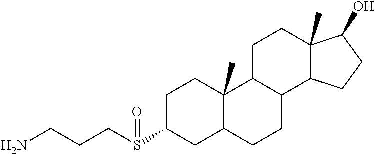 | 183 |
| 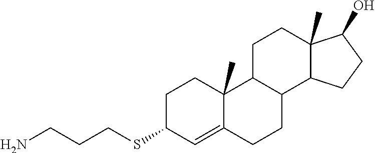 | 184 |
| 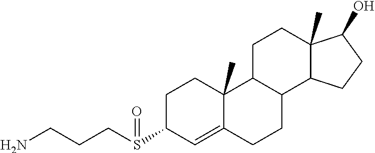 | 185 |
| 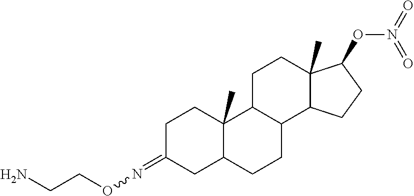 | 186 |
| 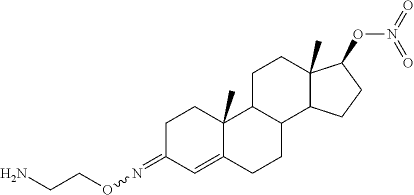 | 187 |
| 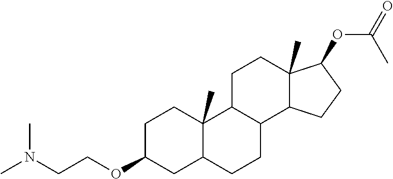 | 188 |
| 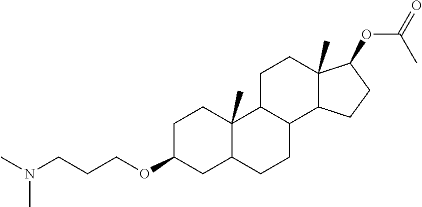 | 189 |
| 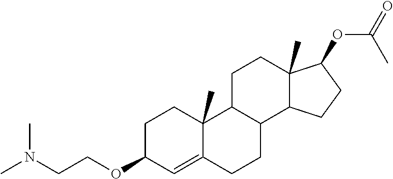 | 190 |
| 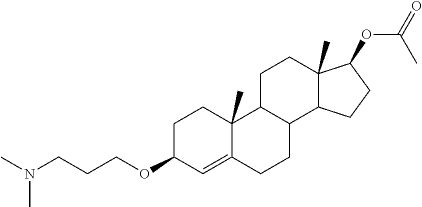 | 191 |
| 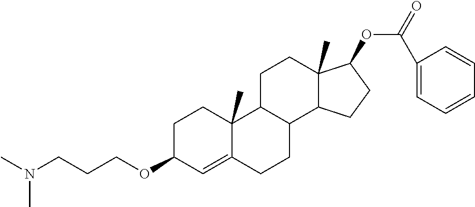 | 192 |
| 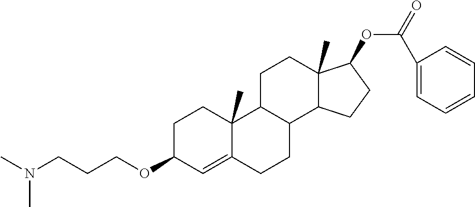 | 193 |
| 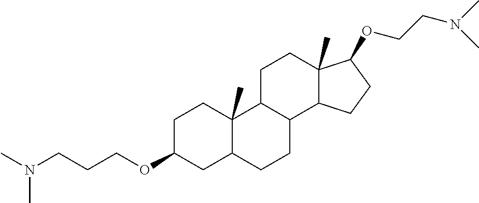 | 194 |
| 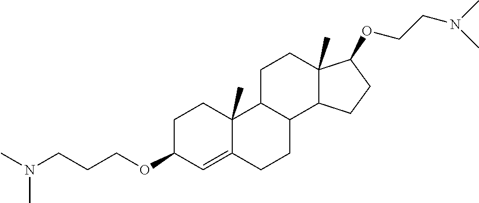 | 195 |
| 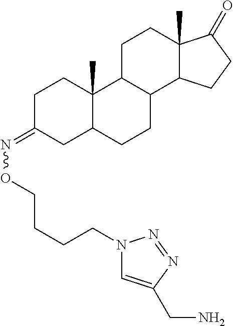 | 196 |
| 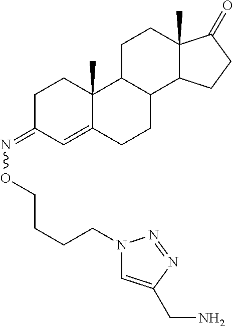 | 197 |

The invention also provides a compound having Formula (Ia)

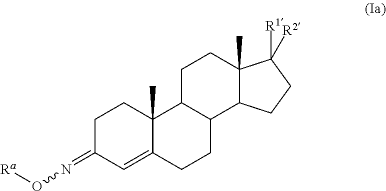

wherein

one of R^{1′} and R^{2′} is H, and the other is —OH; or R^{1′} and R^{2′} together with the carbon to which they are attached form C═O;

R^{a} is aminoalkyl or a 4- to 6-membered heterocyclic ring; wherein said aminoalkyl or said heterocyclic ring is optionally substituted by one or more alkyl.

In one embodiment, one of R^{1′} and R^{2′} is H, and the other is —OH. Another embodiment provides that R^{1′} and R^{2′} together with the carbon to which they are attached form C═O.

One embodiment provides that R^{a} is amino(C₁-C₄)alkyl. Another embodiment provides that R^{a} is a 4- to 6-membered heterocyclic ring.

Compounds of Formula (Ia) include, but are not limited to,

(10R,13S)-3-(2-aminoethoxyimino)-10,13-dimethyl-2,3,7,8,9,10,11,12,13,14,15,16-dodecahydro-1H-cyclopenta[a]phenanthren-17(6H)-one (Compound 12):

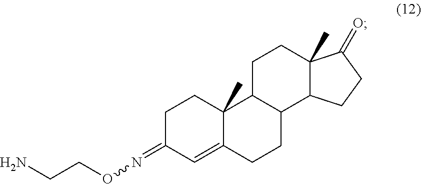

(10R,13S)-10,13-dimethyl-3-(pyrrolidin-3-yloxyimino)-2,3,7,8,9,10,11,12,13,14,15,16-dodecahydro-1H-cyclopenta[a]phenanthren-17(6H)-one (Compound 61):

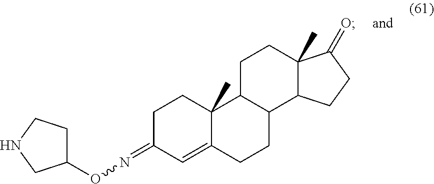

(1S,9aR,11aS)-17-hydroxy-10,13-dimethyl-6,7,8,9,10,11,12,13,14,15,16,17-dodecahydro-1H-cyclopenta[a]phenanthren-3(2H)-one O-2-aminoethyl oxime (Compound 120):

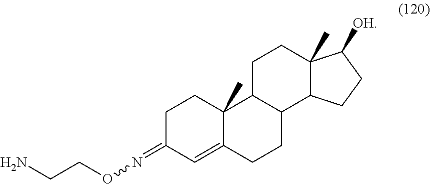

The invention further provides a compound of Formula (Ib):

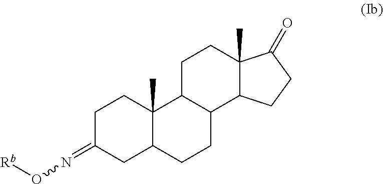

wherein

R^{b} is alkyl, aminoalkyl or a 4- to 6-membered heterocyclic ring;

wherein said alkyl is substituted by a heteroaryl group that is optionally substituted by one or more aminoalkyl;

said aminoalkyl group, each independently, is optionally substituted by one or more alkyl; and

said 4- to 6-membered heterocyclic ring is optionally substituted by one or more alkyl.

In one embodiment, R^{b} is (C₁-C₄)alkyl substituted by a heteroaryl moiety that is further substituted by an amino(C₁-C₄)alkyl group. In another embodiment, R^{b} is amino(C₁-C₄)alkyl. In a certain embodiment, R^{b} is a 4- to 6-membered heterocyclic ring.

Certain compounds of Formula (Ib) include:

(10S,13S)-3-(2-amino ethoxyimino)-10,13-dimethyltetradecahydro-1H-cyclopenta[a]phenanthren-17(2H)-one (Compound 1):

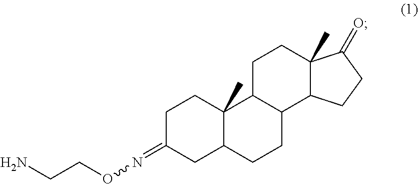

(10S,13S)-10,13-dimethyl-3-(pyrrolidin-3-yloxyimino)tetradecahydro-1H-cyclopenta[a]phenanthren-17(2H)-one (Compound 56):

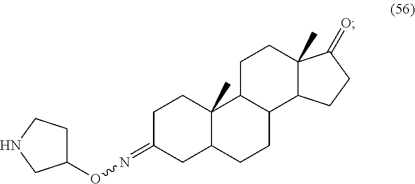

(10S,13S)-3-(2-(4-(aminomethyl)-1H-1,2,3-triazol-1-yl)ethoxyimino)-10,13-dimethyltetradecahydro-1H-cyclopenta[a]phenanthren-17(2H)-one (Compound 31):

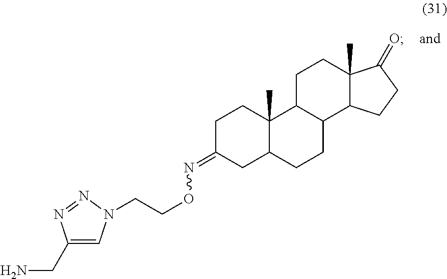

(10S,13S)-3-(2-(4-(aminomethyl)-1H-1,2,3-triazol-1-yl)butoxyimino)-10,13-dimethyltetradecahydro-1H-cyclopenta[a]phenanthren-17(2H)-one (Compound 196):

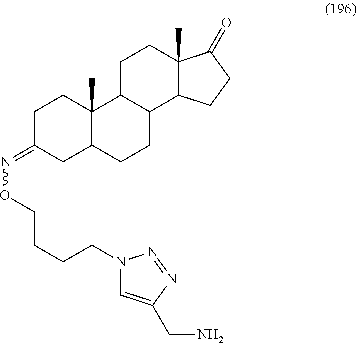

The structures of the compounds of the invention may include asymmetric carbon atoms. Accordingly, the isomers arising from such asymmetry (e.g., all enantiomers and diastereomers) are included within the scope of this invention, unless indicated otherwise. Such isomers can be obtained in substantially pure form by classical separation techniques and/or by stereochemically controlled synthesis.

Naturally occurring or synthetic isomers can be separated in several ways known in the art. Methods for separating a racemic mixture of two enantiomers include chromatography using a chiral stationary phase (see, e.g., “Chiral Liquid Chromatography,” W. J. Lough, Ed. Chapman and Hall, New York (1989)). Enantiomers can also be separated by classical resolution techniques. For example, formation of diastereomeric salts and fractional crystallization can be used to separate enantiomers. For the separation of enantiomers of carboxylic acids, the diastereomeric salts can be formed by addition of enantiomerically pure chiral bases, such as brucine, quinine, ephedrine, strychnine, and the like. Alternatively, diastereomeric esters can be formed with enantiomerically pure chiral alcohols, such as menthol, followed by separation of the diastereomeric esters and hydrolysis to yield the free, enantiomerically enriched carboxylic acid. For separation of the optical isomers of amino compounds, addition of chiral carboxylic or sulfonic acids, such as camphorsulfonic acid, tartaric acid, mandelic acid, or lactic acid can result in formation of the diastereomeric salts.

In certain embodiments, it is believed that a compound of the invention is capable of preventing or treating a neoplasia (e.g., a membrane androgen positive solid tumor or hematological malignancy) in a subject. The compounds of the invention are believed to act as Na⁺K⁺ ATPase inhibitors, which inhibit ligand binding to a membrane androgen receptor, thereby preventing or treating the neoplasia.

In certain instances, it is believed that a compound of the invention binds a Na⁺K⁺ ATPase and inhibits Na⁺K⁺ ATPase activity. A compound of the invention may bind to a membrane androgen receptor and competitively inhibit ligand binding to the receptor. In other instances, it is believed that a compound of the invention induces cell death in a neoplastic cell of the neoplasia. In some embodiments, the neoplasia is a prostate cancer, breast cancer, or colon cancer.

A compound of the invention may also be used in treating or preventing prostate cancer in a subject. The compound may bind and inhibit a Na⁺K⁺ ATPase, and also inhibit ligand binding to the membrane androgen receptor on a prostate cancer cell. In one instance, a compound of the invention may induce cell death (e.g., apoptosis) in a cell of the prostate cancer. In another instance, a compound of the invention may bind the membrane androgen receptor.

### Uses of the Compounds of the Invention

The invention also provides methods for treating a subject for a neoplasia by administering to the subject an effective amount of a compound of the invention. In certain embodiments, the subject is a mammal, in particular a human.

In accordance with the invention, compounds are administered in combination with a pharmaceutically diluent or acceptable carrier. In one embodiment, the compound can be administered using a pharmaceutically acceptable formulation. In advantageous embodiments, the pharmaceutically-acceptable carrier provides sustained delivery of the compound to a subject for at least four weeks after administration to the subject.

In certain embodiments, the compound is administered orally. In other embodiments, the compound is administered intravenously. In yet other embodiments, the compound is administered topically. In still other embodiments, the compound is administered topically or parenterally.

Although dosages may vary depending on the particular indication, route of administration and subject, the compounds are typically administered at a concentration of about 0.1 μg to 20 milligram per kilogram of body weight per day (mg/kg/day) (e.g., 0.1 m/kg to 2 mg/kg, 0.3-3 μm/kg, 0.18-0.54 mg/kg). In other embodiments, the amount varies from about 0.1 mg/kg/day to about 100 mg/kg/day. In still other embodiments, the amount varies from about 0.001 μg to about 100 μg/kg (e.g., of body weight).

Determination of a therapeutically effective amount or a prophylactically effective amount of a compound described herein can readily by one skilled in the art. The dosages may be varied depending upon the requirements of the patient, the severity of the condition being treated and the particular compound being employed. In determining the therapeutically effective amount or dose, and the prophylactically effective amount or dose, a number of factors are considered, including, but not limited to: the specific hyperplastic/neoplastic cell involved; pharmacodynamic characteristics of the particular agent and its mode and route of administration; the desired time course of treatment; the species of mammal; its size, age, and general health; the specific disease involved; the degree of or involvement or the severity of the disease; the response of the individual subject; the particular compound administered; the mode of administration; the bioavailability characteristics of the preparation administered; the dose regimen selected; the kind of concurrent treatment (i.e., the interaction of the compounds of the invention with other co-administered therapeutics); and other relevant circumstances. U.S. Pat. No. 5,427,916, for example, describes method for predicting the effectiveness of antineoplastic therapy in individual patients, and illustrates certain methods which can be used in conjunction with the treatment protocols of the instant invention.

Treatment can be initiated with smaller dosages which are less than the optimum dose of the compound. Thereafter, the dosage should be increased by small increments until the optimum effect under the circumstances is reached. For convenience, the total daily dosage may be divided and administered in portions during the day if desired. A therapeutically effective amount and a prophylactically effective amount of a compound of the invention is expected to vary from about 0.1 μg to 20 milligram per kilogram of body weight per day (mg/kg/day) (e.g., 0.1 μm/kg to 2 mg/kg, 0.3-3 μm/kg, 0.18-0.54 mg/kg). In other embodiments, the amount varies from about 0.1 mg/kg/day to about 100 mg/kg/day. In still other embodiments, the amount varies from about 0.001 μg to about 100 μg/kg (e.g., of body weight).

Compounds which are determined to be effective for the prevention or treatment of neoplasias in animals, e.g., dogs, rodents, may also be useful in treatment of neoplasias in humans. Those skilled in the art of treating neoplasias in humans will know, based upon the data obtained in animal studies, the dosage and route of administration of the compound to humans. In general, the dosage and route of administration in humans are expected to be similar to that in animals.

The identification of those patients who are in need of prophylactic treatment for hyperplastic/neoplastic disease states is well within the ability and knowledge of one skilled in the art. Certain of the methods for identification of patients who are at risk of developing neoplastic disease states which can be treated by the subject method are appreciated in the medical arts, such as family history of the development of a particular disease state and the presence of risk factors associated with the development of that disease state in the subject patient. A clinician skilled in the art can readily identify such candidate patients, by the use of, for example, clinical tests, physical examination and medical/family history.

Another aspect of the invention comprises obtaining the compound of the invention.

### Neoplasia

The invention features methods for inhibiting the proliferation, growth, or viability of a neoplastic cell by contacting the cells with a compound of formula (I) or otherwise described herein. In general, the method includes a step of contacting a neoplastic cell with an effective amount of a compound of the invention. The present method can be performed on cells in culture, e.g., in vitro or ex vivo, or can be performed on cells present in an animal subject, e.g., as part of an in vivo therapeutic protocol. The therapeutic regimen can be carried out on a human or other subject.

The compounds of the invention or otherwise described herein can be tested initially in vitro for their inhibitory effects on the proliferation or survival of neoplastic cells. Examples of cell lines that can be used are lung cancer cell lines (e.g., H460, EKVX, A549), breast cancer cell lines (e.g., MCF7, T47D), CNS cancer cell lines (e.g., SF268, U251, SF295), colon cancer cell lines (e.g., HCT116, HCT15), prostate (e.g., PC-3, DU145), ovarian cancer cell lines (e.g., IGROV1, OVCAR5, OVCAR3, NCI-ADRRES), pancreatic cancer cell lines (e.g., SU8686), renal cancer cell lines (e.g., CAKI), and melanoma cancer cell lines (e.g., LOXIMVI, SKMEL28, MB435).

Alternatively, the antineoplastic activity of compounds of the invention can be tested in vivo using various animal models known in the art. For example, xenographs of human neoplastic cells or cell lines are injected into immunodeficient mice (e.g., nude or SCID) mice. Compounds of the invention are then administered to the mice and the growth and/or metastasis of the tumor is compared in mice treated with a compound of the invention relative to untreated control mice. Agents that reduce the growth or metastasis of a tumor or increase mice survival are identified as useful in the methods of the invention.

The methods discussed herein can be used to inhibit the proliferation of virtually any neoplastic cell. Neoplasia growth is typically uncontrolled and progressive, and occurs under conditions that would not elicit, or would cause cessation of, multiplication of normal cells. Neoplasias can affect a variety of cell types, tissues, or organs, including but not limited to an organ selected from the group consisting of bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasias include cancers, such as acoustic neuroma, acute leukemia, acute lymphocytic leukemia, acute monocytic leukemia, acute myeloblastic leukemia, acute myelocytic leukemia, acute myelomonocytic leukemia, acute promyelocytic leukemia, acute erythroleukemia, adenocarcinoma, angiosarcoma, astrocytoma, basal cell carcinoma, bile duct carcinoma, bladder carcinoma, brain cancer, breast cancer, bronchogenic carcinoma, cervical cancer, chondrosarcoma, chordoma, choriocarcinoma, chronic leukemia, chronic lymphocytic leukemia, chronic myelocytic leukemia, colon cancer, colon carcinoma, craniopharyngioma, cystadenocarcinoma, embryonal carcinoma, endotheliosarcoma, ependymoma, epithelial carcinoma, Ewing's tumor, glioma, heavy chain disease, hemangioblastoma, hepatoma, Hodgkin's disease, large cell carcinoma, leiomyosarcoma, liposarcoma, lung cancer, lung carcinoma, lymphangioendotheliosarcoma, lymphangiosarcoma, macroglobulinemia, medullary carcinoma, medulloblastoma, melanoma, meningioma, mesothelioma, myxosarcoma, neuroblastoma, non-Hodgkin's disease, oligodendroglioma, osteogenic sarcoma, ovarian cancer, pancreatic cancer, papillary adenocarcinomas, papillary carcinoma, pinealoma, polycythemia vera, prostate cancer, rhabdomyosarcoma, renal cell carcinoma, retinoblastoma, schwannoma, sebaceous gland carcinoma, seminoma, small cell lung carcinoma, squamous cell carcinoma, sweat gland carcinoma, synovioma, testicular cancer, uterine cancer, Waldenstrom's fibrosarcoma, and Wilm's tumor.

### Multidrug Resistant Neoplasias

As exemplified by results obtained with compounds of the invention (e.g., compounds 120, 12, 61, 1, 56, 31 and 196), neoplasias that are resistant or refractory to anti-neoplastic therapies are likely to be susceptible to treatment with the compounds delineated herein. Neoplasias that display resistance to a wide variety of chemotherapeutic agents are described as multidrug resistant. Multidrug resistant neoplasias are characterized by their ability to resist treatment with compounds having diverse structures and mechanisms of action. Multidrug resistance is generally related to alterations in a family of proteins known as ATP-binding cassette (ABC) transporters.

Multidrug resistant neoplasias often display increased expression of ATP-binding cassette (ABC) transporters, which function as ATP-dependent efflux pumps. These pumps actively transport a wide array of anti-cancer and cytotoxic drugs out of the cell. In mammals, the superfamily of ABC transporters includes P-glycoprotein (P-gp) transporters (MDR1 and MDR3 genes in human), the MRP subfamily, and bile salt export protein (ABCB11; Cancer Res (1998) 58, 4160-4167), MDR-3 (Nature Rev Cancer (2002) 2, 48-58), lung resistance protein (LRP) and breast cancer resistant protein (BCRP). These proteins can recognize and efflux numerous substrates with unrelated chemical structures, including many chemotherapeutics. Other causes of multidrug resistance have been attributed to changes in topoisomerase II, protein kinase C and specific glutathione transferase enzymes.

In particular embodiments, compounds of the invention (e.g., compounds 120, 12, 61, 1, 56, 31 and 196) are particularly useful for neoplasias showing alterations in the activity or expression of MDR1, MDR2, or P-gP). In particular embodiments, the drug resistance of the tumor is mediated through the overexpression of P-gp. Numerous mechanisms can lead to overexpression of P-gp, including amplification of the MDR-1 gene (Anticancer Res (2002) 22, 2199-2203), increased transcription of the MDR-1 gene (J Clin Invest (1995) 95, 2205-2214; Cancer Lett (1999) 146, 195-199; Clin Cancer Res (1999) 5, 3445-3453; Anticancer Res (2002) 22, 2199-2203), by mutations in the MDR-1 gene (Cell (1988) 53, 519-529; Proc Natl Acad Sci USA (1991) 88, 7289-7293; Proc Natl Acad Sci USA (1992) 89, 4564-4568) and chromosomal rearrangements involving the MDR-1 gene (J Clin Invest (1997) 99, 1947-1957).

Therapeutic agents to which resistance is conferred via the action of P-gp include, but are not limited to: vinca alkaloids (e.g., vinblastine), the anthracyclines (e.g., adriamycin, doxorubicin), the epipodophyllotoxins (e.g., etoposide), taxanes (e.g., paclitaxel, docetaxel), antibiotics (e.g., actinomycin D and gramicidin D), antimicrotubule drugs (e.g., colchicine), protein synthesis inhibitors (e.g., puromycin), toxic peptides (e.g., valinomycin), topoisomerase Inhibitors (e.g., topotecan), DNA intercalators (e.g., ethidium bromide) and anti-mitotics. See WO 99/20791. The methods and pharmaceutical compositions of the present invention are useful for treating tumors resistant to any one or more of above-listed drugs.

In still other embodiments, the methods of the invention are useful for treating resistant or refractory neoplasias, where the resistance is conferred by an alteration in a topoisomerase (e.g., topoisomerase II), protein kinase C and specific glutathione transferase enzyme. Methods of the invention are also useful for the treatment of neoplasias showing resistance to taxanes (e.g., paclitaxel and docetaxel). Such resistance is typically mediated by alterations in tubulin. In other embodiments, compounds delineated herein are useful for treating neoplasias that are refectory to platinum-based chemotherapeutic agents, including carboplatin, cisplatin, oxaliplatin, iproplatin, tetraplatin, lobaplatin, DCP, PLD-147, JM118, JM216, JM335, and satraplatin. Such platinum-based chemotherapeutic agents also include the platinum complexes disclosed in EP 0147926, U.S. Pat. No. 5,072,011, U.S. Pat. Nos. 5,244,919, 5,519,155, 6,503,943 (LA-12/PLD-147), 6,350,737, and WO 01/064696 (DCP).

In sum, the methods and pharmaceutical compositions of the invention are generally useful for treating resistant and/or refractory neoplasias to any one or more of drugs known in the art or described herein. In particular embodiments, the methods and compositions of the invention are useful for the treatment of patients having end-stage disease, which includes patients for whom no effective therapeutic regimen exists or patients identified as having less than about 3, 6, 9 or 12 months to live.

### Combination Therapies

In certain embodiments, the compounds of the invention are administered in combination with any other standard anti-neoplasia therapy or conventional chemotherapeutic agent, such as an alkylating agent; such methods are known to the skilled artisan and described in Remington's Pharmaceutical Sciences by E. W. Martin. For example, if desired, agents of the invention (e.g., compounds 120, 12, 61, 1, 56, 31, and 196) are administered in combination with any conventional anti-neoplastic therapy, including but not limited to, surgery, radiation therapy, or chemotherapy. Conventional chemotherapeutic agents include, but are not limited to, abiraterone, alemtuzumab, altretamine, aminoglutethimide, amsacrine, anastrozole, azacitidine, bleomycin, bicalutamide, busulfan, capecitabine, carboplatin, carmustine, celecoxib, chlorambucil, 2-chlorodeoxyadenosine, cisplatin, colchicine, cyclophosphamide, cytarabine, cytoxan, dacarbazine, dactinomycin, daunorubicin, docetaxel, doxorubicin, epirubicin, estramustine phosphate, etodolac, etoposide, exemestane, floxuridine, fludarabine, 5-fluorouracil, flutamide, formestane, gemcitabine, gentuzumab, goserelin, hexamethylmelamine, hydroxyurea, hypericin, ifosfamide, imatinib, interferon, irinotecan, letrozole, leuporelin, lomustine, mechlorethamine, melphalen, mercaptopurine, 6-mercaptopurine, methotrexate, mitomycin, mitotane, mitoxantrone, nilutamide, nocodazole, paclitaxel, pentostatin, procarbazine, raltitrexed, rituximab, rofecoxib, streptozocin, tamoxifen, temozolomide, teniposide, 6-thioguanine, topotecan, toremofine, trastuzumab, vinblastine, vincristine, vindesine, and vinorelbine. In particular embodiments, a combination of the invention comprises any one or more of the following: vinca alkaloids (e.g., vinblastine), taxanes (e.g., paclitaxel, docetaxel), epothilones (e.g., ixabepilone), antifolates (e.g., Methotrexate), purine analogs (e.g., fludarabine), pyrimidine analogs (e.g., gemcitabine), DNA intercalators (e.g., ethidium bromide), topoisomerase Inhibitors (e.g., topotecan), alkylating agents (e.g., carmustine, bendamustine), platinum-based agents (e.g., cisplatin, oxaliplatin), receptor antagonists (e.g., atrasentan), hormone agents (e.g. anti-androgens, aromatase inhibitors), anthracyclines (e.g., adriamycin, doxorubicin), epipodophyllotoxins (e.g., etoposide), antibiotics (e.g., actinomycin D and gramicidin D), antimicrotubule drugs (e.g., colchicine), protein synthesis inhibitors (e.g., puromycin), toxic peptides (e.g., valinomycin), enzyme inhibitors (e.g. CDK inhibitors) and anti-mitotics.

The compounds can be administered concurrently or sequentially with chemotherapy. Alternatively, they can be administered using specific administration regimens. For example, to treat a prostate tumor xenograft in mice, the compounds of the invention could be administered intraperitoneally (ip) daily for 5 consecutive days followed by 2 days rest for 4 weekly cycles in total [QdX5; 2)×4], whereas a chemotherapeutic drug (e.g. a taxane) can be administered (ip) every 4 days for a total of 3 injections (Q4D×3). Alternatively, in colon cancer xenograft models in mice, the compounds of the invention could be administered via implanted, pre-filled pumps (e.g. Alzet pumps) continuously delivering the compound at a pre-determined rate, whereas the chemotherapeutic agent (e.g. irinotecan) can be administered (ip) via a Q4D×3 scheme.

### Pharmaceutical Compositions

The invention also provides pharmaceutical compositions for the treatment of a neoplasia, comprising an effective amount a compound of the invention and a pharmaceutically acceptable carrier. In particular embodiments, compositions of the invention comprise a compound described herein in combination with a conventional chemotherapeutic agent. In still other embodiments, such compositions are labeled for the treatment of cancer. In a further embodiment, the effective amount is effective to reduce the growth, proliferation, or survival of a neoplastic cell or to otherwise treat or prevent a neoplasia in a subject, as described herein.

In an embodiment, the compound is administered to the subject using a pharmaceutically-acceptable formulation. In certain embodiments, these pharmaceutical compositions are suitable for oral or parenteral administration to a subject. In still other embodiments, as described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound. In certain embodiments, the subject is a mammal, e.g., a primate, e.g., a human.

The methods of the invention further include administering to a subject a therapeutically effective amount of a compound in combination with a pharmaceutically acceptable excipient. The phrase “pharmaceutically acceptable” refers to those compounds of the invention, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable excipient” includes pharmaceutically-acceptable material, composition or vehicle, such as a liquid or solid filler, diluent, carrier, solvent or encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Compositions containing a compound(s) include those suitable for oral, nasal, topical (including buccal and sublingual), rectal, vaginal, aerosol and/or parenteral administration. The compositions may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these compositions include the step of bringing into association a compound(s) with the carrier and, optionally, one or more accessory ingredients. In general, the formulations are prepared by uniformly and intimately bringing into association a compound with liquid carriers, or finely divided solid carriers, or both, and then, if necessary, shaping the product.

Compositions of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound(s) as an active ingredient. A compound may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered active ingredient moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compound(s) include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

In addition to inert diluents, the oral compositions can include adjuvants, such as wetting agents, emulsifying and suspending agents, sweetening, flavoring, coloring, perfuming and preservative agents.

Suspensions, in addition to the active compound(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of the invention for rectal or vaginal administration may be presented as a suppository, which may be prepared by mixing one or more compound(s) with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Compositions of the present invention which are suitable for vaginal administration also include pessaries, tampons, creams, gels, pastes, foams or spray formulations containing such carriers as are known in the art to be appropriate.

Dosage forms for the topical or transdermal administration of a compound(s) include powders, sprays, ointments, pastes, creams, lotions, gels, solutions, patches and inhalants. The active compound(s) may be mixed under sterile conditions with a pharmaceutically-acceptable carrier, and with any preservatives, buffers, or propellants which may be required.

The ointments, pastes, creams and gels may contain, in addition to compound(s) of the present invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound(s), excipients, such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The compound(s) can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically-acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids, such as glycine, buffers, salts, sugars or sugar alcohols. Aerosols generally are prepared from isotonic solutions.

Transdermal patches have the added advantage of providing controlled delivery of a compound(s) to the body. Such dosage forms can be made by dissolving or dispersing the agent in the proper medium. Absorption enhancers can also be used to increase the flux of the active ingredient across the skin. The rate of such flux can be controlled by either providing a rate controlling membrane or dispersing the active ingredient in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compound(s) in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of compound(s) in biodegradable polymers, such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compound(s) are administered as pharmaceuticals, to humans and animals, they can be given per se or as a pharmaceutical composition containing, for example, 0.1 to 99.5% (more preferably, 0.5 to 90%) of active ingredient in combination with a pharmaceutically-acceptable carrier.

Regardless of the route of administration selected, the compound(s), which may be used in a suitable hydrated form, and/or the pharmaceutical compositions of the present invention, are formulated into pharmaceutically-acceptable dosage forms by conventional methods known to those of skill in the art.

Actual dosage levels and time course of administration of the active ingredients in the pharmaceutical compositions of this invention may be varied so as to obtain an amount of the active ingredient which is effective to achieve the desired therapeutic response for a particular patient, composition, and mode of administration, without being toxic to the patient. An exemplary dose range is from about 0.1 μg to 20 milligram per kilogram of body weight per day (mg/kg/day) (e.g., 0.1 m/kg to 2 mg/kg, 0.3-3 m/kg, 0.18-0.54 mg/kg). In other embodiments, the amount varies from about 0.1 mg/kg/day to about 100 mg/kg/day. In still other embodiments, the amount varies from about 0.001 μg to about 100 μg/kg (e.g., of body weight). Ranges intermediate to the above-recited values are also intended to be part of the invention.

### Kits

The invention provides kits for the treatment or prevention of neoplasia. In one embodiment, the kit includes a therapeutic or prophylactic composition containing an effective amount of a compound of the invention in unit dosage form. In some embodiments, a compound of the invention is provided in combination with a conventional chemotherapeutic agent. In other embodiments, the kit comprises a sterile container which contains a therapeutic or prophylactic composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired a compound of the invention is provided together with instructions for administering the compound to a subject having or at risk of developing neoplasia. The instructions will generally include information about the use of the composition for the treatment or prevention of neoplasia. In other embodiments, the instructions include at least one of the following: description of the therapeutic agent; dosage schedule and administration for treatment or prevention of ischemia or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

### EXEMPLIFICATION OF THE INVENTION

The following examples are put forth so as to provide those of ordinary skill in the art with a complete disclosure and description of how to make and use the assay, screening, and therapeutic methods of the invention, and are not intended to limit the scope of what the inventors regard as their invention.

### I. Chemical Examples

### Synthesis and Methods of Preparation

Compounds of the invention can be synthesized by methods described in this section, the examples, and the chemical literature.

### A. Preparation of Intermediates

### 2-Aminoethoxy-amine dihydrochloride (Intermediate (I))

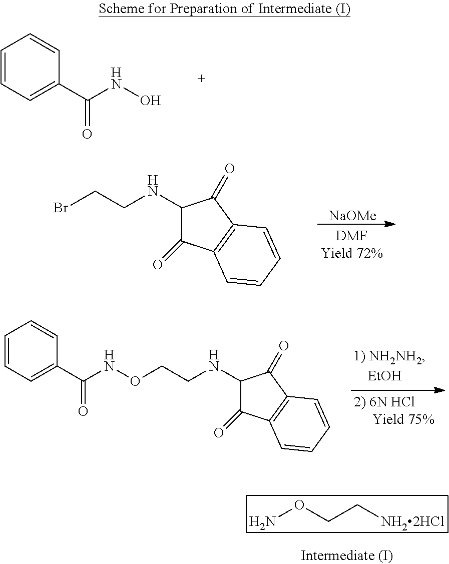

Intermediate (I) was prepared according to the scheme described above. The detailed reaction procedures were described in M. C. Pankaskie et al., An Improved Synthetic Route to Aminoxypropylamine (APA) and Related Homologs, Synthetic Communications vol 19 (3&4), pages 339-344 (1989).

### 3(R)-Pyrrolidinyloxyamine dihydrochloride (Intermediate (II))

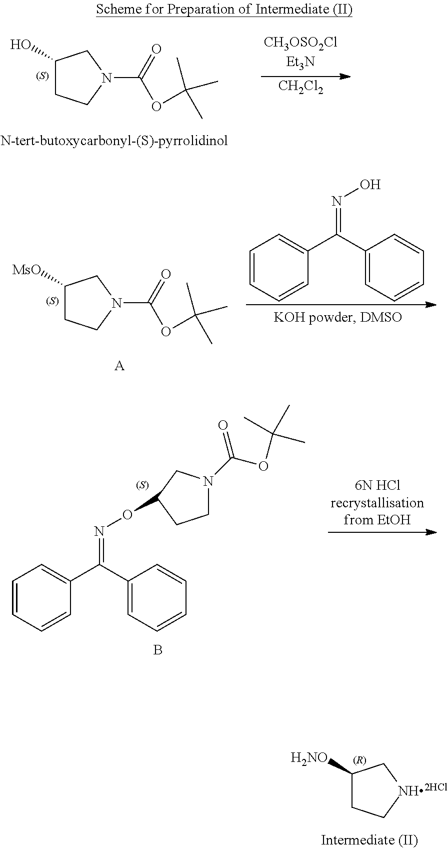

To a solution of N-tert-butoxycarbonyl-(S)-pyrrolidinol (10.0 g) and triethylamine (8.2 mL) in CH₂Cl₂ (150 mL) at 0° C., methanesulfonyl chloride (4.34 mL) was added. After stirring at room temperature for 3 h, the reaction mixture was poured into ice/water and extracted with CH₂Cl₂. The organic phase was washed with 5% aqueous NaHCO₃, water, brine, dried and evaporated to dryness to give an oil which solidified after standing overnight in the refrigerator. The solid was triturated with Et₂O to give N-tert-butoxycarbonyl-(S)-3-pyrrolidinyl methansulfonate (13.0 g, 92%) (Compound A) as a light yellow solid. ¹H-NMR (300 MHz, DMSO-d₆, ppm from TMS): δ 5.23 (1H, m), 3.60-3.10 (4H, m), 3.23 (3H, s), 2.11 (2H, m), 1.39 (9H, s).

To a suspension of KOH powder (4.86 g) in DMSO (250 mL) under vigorous stirring, benzophenone oxime (7.86 g) was added. After stirring at room temperature for 30 min, a solution of N-tert-butoxycarbonyl-(S)-3-pyrrolidinyl methansulfonate (10 g) (Compound A) in DMSO (70 mL) was added. After 18 h at room temperature the reaction was poured into iced water (900 mL) and extracted with Et₂O. The combined organic layers were washed with water, brine, dried and the solvent evaporated. Benzophenone O-[(R)-3-pyrrolidinyl]oxime (Compound B) was obtained (13.0 g, 96%) as a white solid and used without purification in the next step. ¹H-NMR (300 MHz, DMSO-d₆, ppm from TMS): δ 7.50-7.20 (10H, m), 4.84 (1H, m), 3.50-3.00 (4H, m), 2.01 (2H, m), 1.38 (9H, s).

Compound B (13.0 g) was suspended in 6N HCl (250 mL) and the mixture was refluxed for 2 h. After cooling, the reaction was extracted with Et₂O. The aqueous layer was evaporated to give a crude brown solid which was treated with 0.34 g of activated carbon in absolute EtOH (255 mL) at reflux for 2 h. The solid obtained after evaporation was crystallized with 96% EtOH (40 mL) to give the 3(R)-pyrrolidinyloxyamine dihydrochloride (Intermediate (II)) (2.98 g, 72%), as an off white solid. ¹H-NMR (300 MHz, DMSO-d₆, ppm from TMS): δ 11.22 (3H, bb), 9.74 (1H, bb), 9.54 (1H, bb), 4.98 (1H, m), 3.60-3.00 (4H, m), 2.40-2.00 (2H, m).

### (1-(2-(Aminooxy)ethyl)-1H-1,2,3-triazol-4-yl)methanamine dihydrochloride (Intermediate (III))

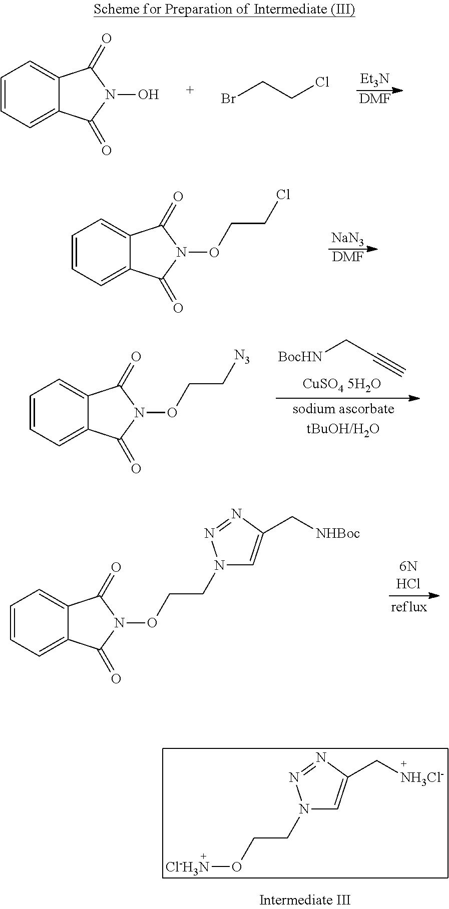

To a stirred solution of N-hydroxyphthalimide (2.0 g, 12.26 mmol) in DMF (15 mL) Et₃N (3.8 mL, 26.97 mmol) and 1-bromo-2-chloroethane (4.79 mL, 57.6 mmol) were added and the mixture was stirred overnight at room temperature. The solid was filtered and the precipitate was washed with DMF. The filtrate was diluted with AcOEt and the organic layer was extracted with 1M HCl (×2) and brine. The organic layer was dried over anhydrous Na₂SO₄, filtered and evaporated under reduced pressure at 60° C. for the removal of the excess alkyl bromide to afford 2-(2-chloroethoxy)isoindoline-1,3-dione 2.7 g (97%) as a white solid. ¹H-NMR (300 MHz, CDCl₃) δ 7.85-7.73 (4H, m), 4.44 (2H, t, J=6.2 Hz), 3.83 (2H, t, J=6.2 Hz).

To a stirred solution of 2-(2-chloroethoxy)isoindoline-1,3-dione (2.6 g, 11.5 mmol) in DMF (44 ml), NaN₃ (6.65 g, 102.3 mmol) was added and the reaction mixture was stirred for 3 days at 45° C. The reaction was diluted with CHCl₃ and H₂O, and the organic layer was extracted with CHCl₃ (×2) and the combined organic phases were dried over anhydrous Na₂SO₄, filtered and evaporated under reduced pressure to afford 2-(2-azidoethoxy)isoindoline-1,3-dione 2.67 g (100%) as a white solid. ¹H-NMR (600 MHz, CDCl₃) δ 7.85-7.73 (4H, m), 4.33 (2H, bs), 3.64 (2H, bs). ¹³C-NMR (75 MHz, CDCl₃): δ 163.3, 134.6, 128.7, 123.6, 76.7, 49.4.

To a stirred solution of 2-(2-azidoethoxy)isoindoline-1,3-dione (697 mg, 3.0 mmol) in t-BuOH (24 mL) and H₂O (24 mL), Boc-propargylamine (466 mg, 3.0 mmol), copper sulfate (225 mg, 0.9 mmol) and sodium ascorbate (357 mg, 1.8 mmol) were added. At that point the reaction mixture appeared as a yellowish slurry, while after 1 h the reaction mixture was clear and after 4 h it turned green, at which point the reaction was complete. DCM and H₂O were added, the layers were separated and the organic phase was extracted with NH₄OH and brine (×2) and subsequently was dried over anhydrous Na₂SO₄, filtered and evaporated under reduced pressure, to afford tert-butyl (1-(2-(1,3-dioxoisoindolin-2-yloxy)ethyl)-1H-1,2,3-triazol-4-yl)methyl-carbamate 1.011 g (87%). ¹H-NMR (600 MHz, CDCl₃) δ 8.08 (1H, s), 7.85-7.73 (4H, m), 5.09 (1H, bs), 4.73 (2H, t, J=4.7 Hz), 4.58 (2H, t, J=4.6 Hz), 4.33 (2H, d, J=5.7 Hz), 1.44 ppm (12H, s).

A suspension of tert-butyl (1-(2-(1,3-diox ois oindolin-2-yloxy)ethyl)-1H-1,2,3-triazol-4-yl)methylcarbamate (0.59 g, 1.5 mmol) in 6N HCl (4 mL) was stirred at 85° C. for 15 min, and then was cooled to room temperature and was extracted with Et₂O. The aqueous phase was evaporated, EtOH was added and the mixture was evaporated under reduced pressure until H₂O was removed. Subsequently, absolute EtOH was added and a white solid precipitated, which was filtered and washed with cold EtOH to afford 265 mg (77%) of (1-(2-(Aminooxy)ethyl)-1H-1,2,3-triazol-4-yl)methanamine dihydrochloride (Intermediate (III)). ¹H-NMR (300 MHz, CD₃OD) δ 8.17 (1H, s), 4.82 (2H, t, J=4.8 Hz), 4.52 (2H, t, J=4.8 Hz), 4.26 (2H, s).

### (1-(2-(Aminooxy)butyl)-1H-1,2,3-triazol-4-yl)methanamine dihydrochloride (Intermediate (IV))

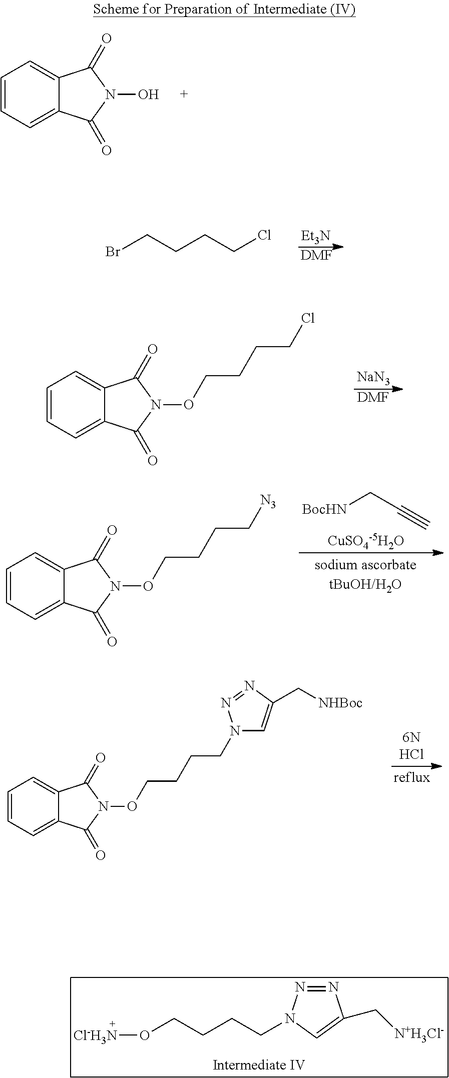

To a stirred solution of N-hydroxyphthalimide (2.0 g, 12.26 mmol) in DMF (15 mL) Et₃N (3.8 mL, 26.97 mmol) and 1-bromo-4-chlorobutane (6.64 mL, 57.6 mmol) were added. The reaction mixture was stirred overnight at room temperature and subsequently the mixture was filtered and the precipitate was washed with DMF. The filtrate was diluted with AcOEt and was extracted with 1M HCl (×2) and brine. The organic layer was dried over anhydrous Na₂SO₄, filtered and evaporated under reduced pressure at 60° C. for the removal of the excess alkyl bromide to afford 2-(2-chlorobutoxy)isoindoline-1,3-dione 3.11 g (100%) as colorless oil. ¹H-NMR (300 MHz, CDCl₃) δ 7.85-7.73 (4H, m), 4.24 (2H, t, J=6.0 Hz), 3.67 (2H, t, J=6.2 Hz), 2.05-1.92 (4H, m).

To a stirred solution of 2-(2-chlorobutoxy)isoindoline-1,3-dione (3.11 g, 12.26 mmol) in DMF, NaN₃ (7.97 g, 122.6 mmol) was added and the reaction mixture was stirred for 3 days at 45° C. The reaction was diluted with CHCl₃ and H₂O, and the organic layer was extracted with CHCl₃ (×2) and the combined organic phases were dried over anhydrous Na₂SO₄, filtered and evaporated under reduced pressure. The residue was purified by column chromatography elution solvent AcOEt/cyclohexane 10/90→25/75 to afford 2-(2-azidobutoxy)isoindoline-1,3-dione 3.15 g (99%) as colorless oil. ¹H-NMR (300 MHz, CDCl₃): δ 7.85-7.73 (4H, m), 4.23 (2H, t, J=6.0 Hz), 3.41 (2H, t, J=6.2 Hz), 1.87 (4H, m).

To a stirred solution of 2-(2-azidobutoxy)isoindoline-1,3-dione (781 mg, 3.0 mmol) in tBuOH (25 mL) and H₂O (25 mL), Boc-propargylamine (466 mg, 3.0 mmol), copper sulfate (225 mg, 0.9 mmol) and sodium ascorbate (357 mg, 1.8 mmol) were added. At that point the reaction mixture appeared as a yellowish slurry, while after 1 h the reaction mixture was clear and after 4 h it turned green, at which point the reaction was complete. DCM and H₂O were added, the layers were separated and the organic phase was extracted with NH₄OH and brine (×2) and subsequently was dried over anhydrous Na₂SO₄, filtered and evaporated under reduced pressure, to afford tert-butyl (1-(2-(1,3-dioxoisoindolin-2-yloxy)butyl)-1H-1,2,3-triazol-4-yl)methylcarbamate 1.126 g (90%). ¹H-NMR (300 MHz, CDCl₃): δ 7.83-7.73 (4H, m), 7.59 (1H, s), 5.19 (1H, bs), 4.48 (2H, t, J=6.9 Hz), 4.37 (2H, d, J=5.1 Hz), 4.21 (2H, t, J=5.8 Hz), 2.20 (2H, m), 1.75 (2H, m); ¹³C-NMR (CDCl₃) δ 163.5, 155.8, 147.9, 134.5, 128.7, 123.5, 122.0, 79.5, 77.3, 49.6, 36.1, 28.3, 26.7, 25.0.

A suspension of tert-butyl (1-(2-(1,3-dioxoisoindolin-2-yloxy)butyl)-1H-1,2,3-triazol-4-yl)methylcarbamate (1.12 g, 2.70 mmol) in 6N HCl (5 mL) at 75° C. is stirred for 25 min, and then was cooled to room temperature and was extracted with Et₂O. The aqueous phase was evaporated, EtOH was added and the mixture was evaporated under reduced pressure until H₂O was removed. Subsequently, absolute EtOH was added and a white solid precipitated, which was filtered and washed with cold EtOH to afford 535 mg (77%) of (1-(2-(aminooxy)ebutyl)-1H-1,2,3-triazol-4-yl)methanamine dihydrochloride (Intermediate (IV)). ¹H-NMR (300 MHz, CD₃OD) δ 8.15 (1H, s), 4.50 (2H, t, J=7.0 Hz), 4.26 (2H, s), 4.10 (2H, t, J=6.1 Hz), 2.05 (2H, m), 1.72 (2H, m).

### B. Preparation of Compounds of The Invention

Compounds of the invention, including Compounds 120, 12, 61, 1, 56, 31, and 196 (Table 2) can be synthesized by methods described in this section, the examples, and the chemical literature, which in no way should be construed as being further limiting.

TABLE 2

| | |
|---|---|
| 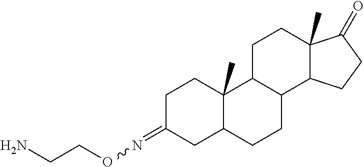 | (Compound 1) |
| 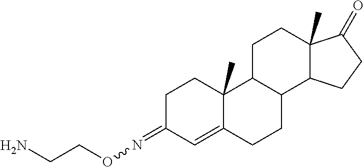 | (Compound 12) |
| 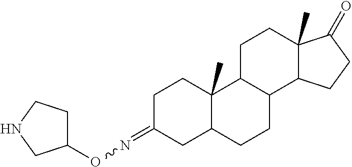 | (Compound 56) |
| 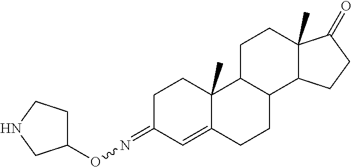 | (Compound 61) |
| 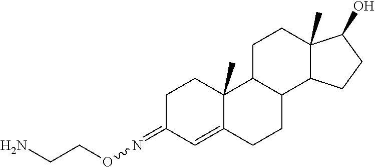 | (Compound 120) |
| 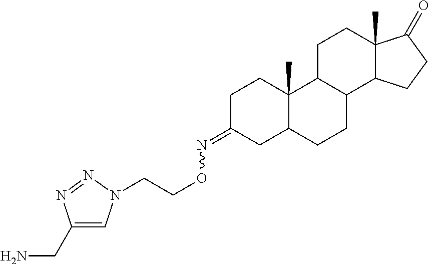 | (Compound 31) |
| 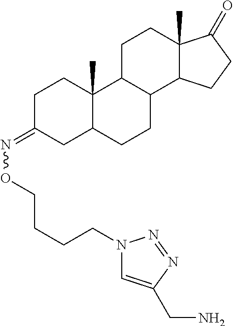 | (Compound 196) |

### Example 1

### Synthesis of HCl salt of 17-Hydroxy-10,13-dimethyl-1,2,6,7,8,9,10,11,12,13,14,15,16,17-tetradecahydro-cyclopenta[a]phenanthren-3-one O-(2-amino-ethyl)-oxime (Hydrochloride salt of Compound (120))

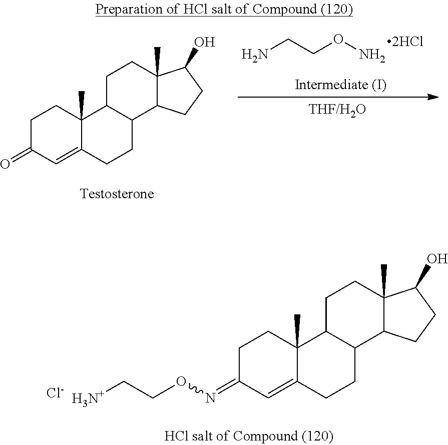

To a solution of testosterone (0.4 mmol) in THF (3 mL), a solution of 2-aminoethoxyamine dihydrochloride (Intermediate (I)) (0.4 mmol) in water (1.5 mL) was added. After 2 hours at room temperature, NaCl (3 mmol) was added and the reaction was stirred for 15 min. The mixture was extracted with THF and the combined organic phases were washed with brine, dried over Na₂SO₄ and evaporated. The residue was treated with H₂O and extracted with DCM (3×). The combined organic extracts were washed with brine, dried over Na₂SO₄ and evaporated to dryness. The desired product was obtained as a solid after crystallisation with AcOEt and filtration. The solid was triturated with Et₂O to afford the HCl salt of 17-hydroxy-10,13-dimethyl-1,2,6,7,8,9,10,11,12,13,14,15,16,17-tetradecahydro-cyclopenta[a]phenanthren-3-one O-(2-amino-ethyl)-oxime (Hydrochloride salt of Compound (120)) in 33% yield as a white solid. ¹H-NMR (600 MHz, CD₃OD): δ 6.43 (s, 0.4H), 5.73 (s, 0.6H), 4.20 (s, 1.2H), 4.18 (s, 0.8H), 3.55 (t, 1H, J=8.6 Hz), 3.22 (bs, 2H), 3.00 (m, 1H), 2.40-1.20 (m, 17H), 1.13 and 1.09 (s, 3H), 1.07-0.78 (m, 3H), 0.76 (s, 3H). ¹³C-NMR (300 MHz, CDCl₃): δ 157.7, 156.7, 116.9, 81.7, 71.2, 54.0, 53.8, 50.6, 42.8, 41.4, 37.9, 36.5, 35.8, 34.6, 32.4, 31.7, 30.4, 24.7, 23.4, 20.9, 19.5, 18.0, 17.8, 11.0. MS (ESI): (m/z) 347.6 [(M-C1]±(100).

### Example 2

### Synthesis of HCl salt of 10,13-Dimethyl-1,6,7,8,9,10,11,12,13,14,15,16-dodecahydro-2H-cyclopenta[a]phenanthrene-3,17-dione 3-[O-(2-amino-ethyl)-oxime (Hydrochloride salt of Compound (12))

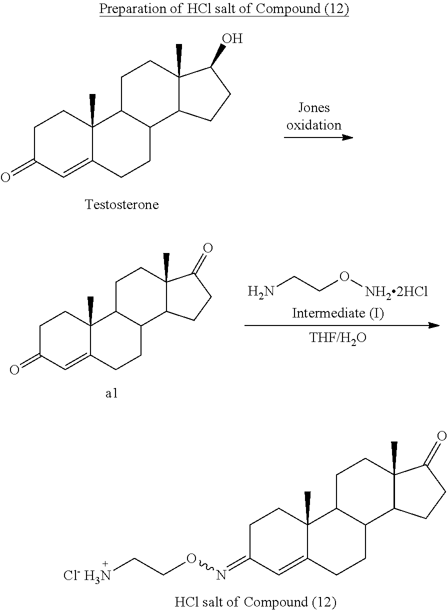

To a solution of testosterone (1.7 mmol) in acetone (10 mL) was added an excess of Jones reagent (˜2 mL) dropwise, maintaining the temperature at 0° C. After completion of the reaction (monitored by TLC), iPrOH was added and, after further 10 min, the suspension was filtered through a short plug of silica, washed with acetone and the filtrate evaporated to dryness. The residue was treated with H₂O and extracted with EtOAc (3×25 mL). The combined organic extracts were washed with H₂O, 5% aqueous NaHCO₃ solution, H₂O, dried over Na₂SO₄ and evaporated to dryness to give the desired product as a white solid. The product was subjected to a short silica column to afford compound a1 as a white solid in quantitative yield. ¹H-NMR (600 MHz, CDCl₃): δ 5.75 (s, 1H) 2.52-1.22 (m, 17H), 1.21 (s, 3H), 1.15-0.95 (m, 2H) 0.92 (s, 3H).

Prepared in 34% yield as described in Example 1 for compound (120) starting from 4-androstene-3,17-dione (compound a1) and 2-aminoethoxyamine dihydrochloride (Intermediate (I)). The crude product was triturated with Et₂O to yield the HCl salt of the title compound (Compound (12)). ¹H-NMR (600 MHz, CDCl₃): δ 6.46 (s, 0.3H), 5.79 (s, 0.7H), 4.29 (bs, 2H), 3.33 (bs, 2H), 3.00 (m, 1H), 2.50-1.12 (m, 17H), 1.11 (s, 0.9H), 1.07 (s, 2.1H) 1.05 (m, 1H) 0.89 (s, 3H). ¹³C-NMR (300 MHz, CDCl₃): δ 220.7, 220.5, 160.3, 158.1, 156.2, 155.1, 117.2, 111.1, 68.7, 60.4, 53.9, 53.7, 51.0, 50.9, 47.5, 40.4, 39.0, 38.0, 35.8, 35.3, 34.5, 32.2, 31.4, 30.9, 21.8, 20.6, 19.6, 18.0, 17.7, 13.7.

### Example 3

### Synthesis of HCl salt of 10,13-Dimethyl-1,6,7,8,9,10,11,12,13,14,15,16-dodecahydro-2H-cyclopenta[a]phenanthrene-3,17-dione 3-(O-pyrrolidin-3-yl-oxime) (Hydrochloride salt of Compound (61))

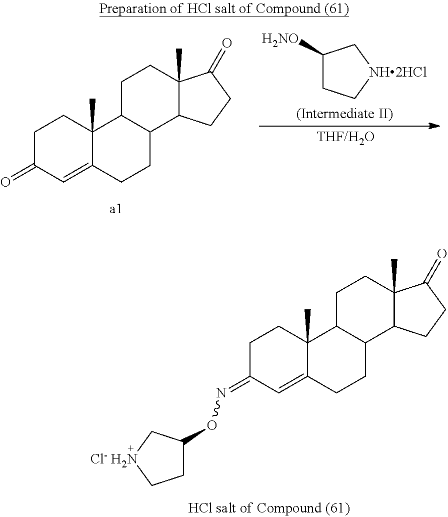

To a solution of the 4-androstene-3,17-dione (compound a1) (0.4 mmol) in THF (3 mL), a solution of 3-(R)-pyrrolidinyloxyamine dihydrochloride (Intermediate (II)) (0.4 mmol) in water (1.5 mL) was added. After 2 hours at room temperature, NaCl (3 mmol) was added and the reaction was stirred for 15 min. The mixture was extracted with THF and the combined organic phases were washed with brine, dried over Na₂SO₄ and evaporated. The residue was treated with H₂O and extracted with DCM (3×). The combined organic extracts were washed with brine, dried over Na₂SO₄ and evaporated to dryness. The hydrochloride salt of compound 61 was obtained as a white solid after crystallisation from AcOEt in 40% yield. ¹H-NMR (600 MHz, CDCl₃): δ 10.08 (bs, 1H), 9.62 (bs, 1H), 6.40 (s, 0.4H), 5.78 (s, 0.6H), 4.85 (bs, 1H), 3.45 (m, 4H), 2.91 (m, 1H), 2.50-1.13 (m, 19H), 1.11 (s, 1.2H), 1.08 (s, 1.8H) 1.00 (m, 1H) 0.90 (s, 3H). ¹³C-NMR (300 MHz, CDCl₃): δ 220.7, 220.6, 158.3, 156.2, 117.2, 111.2, 79.9, 79.6, 77.2, 53.9, 53.7, 51.0, 50.9, 49.8, 47.5, 38.0, 35.8, 35.3, 32.3, 31.4, 30.9, 24.6, 21.8, 20.6, 20.4, 19.6, 17.9, 17.7, 13.7.

### Example 4

### Synthesis of HCl salt of 10,13-Dimethyl-tetradecahydro-cyclopenta[a]phenanthrene-3,17-dione 3-[O-(2-amino-ethyl)-oxime] (Hydrochloride salt of Compound (1)

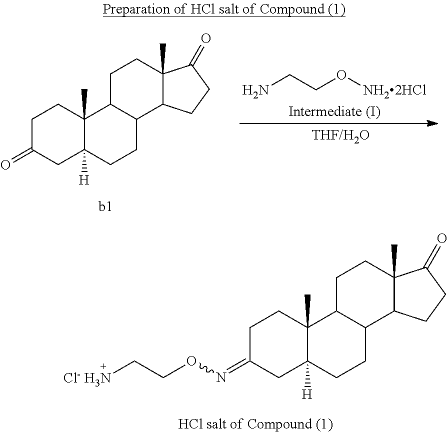

The HCl salt of Compound (1) was prepared in 37% yield as described in Example 1 for the preparation of compound (120), starting from 5α-androstane-3,17-dione (compound (b1)) and 2-aminoethoxyamine dihydrochloride (Intermediate (I)). ¹H-NMR (600 MHz, CDCl₃): δ 8.00 (bs, 3H), 4.25 (t, 2H, J=4.2 Hz), 3.33 (bs, 2H), 3.17 (m, 1H), 2.95 (m, 1H), 2.50-0.90 (m, 19H), 0.91 (s, 3H), 0.87 (s, 3H), 0.75 (m, 1H). ¹³C-NMR (300 MHz, CDCl₃): δ 221.0, 162.31, 162.29, 68.2, 54.0, 53.9, 51.3, 47.73, 47071, 46.3, 45.2, 38.2, 37.1, 36.2, 35.8, 35.0, 31.5, 30.6, 28.5, 28.3, 28.0, 21.8, 21.4, 20.5, 20.4, 13.8, 11.5, 11.3; MS (ESI): (m/z) 347.0 [M-Cl]⁺ (100).

### Example 5

### Synthesis of HCl salt of 10,13-Dimethyl-tetradecahydro-cyclopenta[a]phenanthrene-3,17-dione 3-[O-(2-amino-ethyl)-oxime] (Hydrochloride salt of Compound 56)

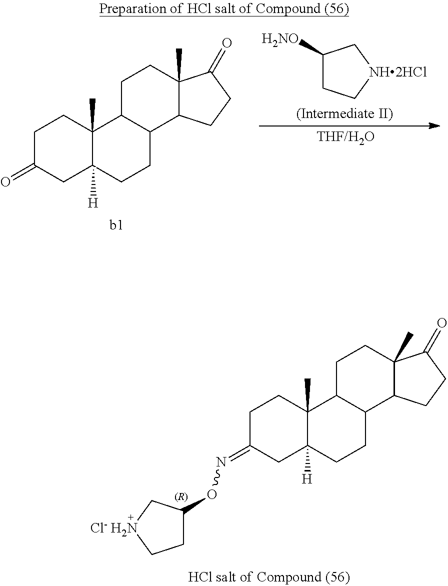

Following the procedure described in Example 3 and starting from 5α-androstane-3,17-dione (compound (b1)) and 3-(R)-pyrrolidinyloxyamine dihydrochloride (Intermediate (II) the HCl salt of compound (56) was obtained in 48% yield as a white powder. ¹H-NMR (300 MHz, CDCl₃): δ 9.99 (bs, 1H), 9.65 (bs, 1H), 4.81 (bs, 1H), 3.55-3.38 (m, 4H), 3.12 (m, 1H), 2.89 (m, 1H), 2.87-1.00 (m, 21H), 0.90 and 0.89 (s and s, 3H), 0.85 (s, 3H), 0.72 (m, 1H). ¹³C-NMR (300 MHz, CDCl₃): δ 221.1, 221.0, 162.4, 162.1, 79.5, 79.4, 54.0, 53.9, 51.3, 51.2, 49.7, 49.6, 47.7, 46.6, 45.0, 44.0, 43.9, 38.2, 37.3, 36.2, 36.1, 35.8, 34.9, 34.3, 31.5, 30.6, 30.4, 28.4, 28.3, 28.1, 27.7, 21.7, 21.5, 20.5, 20.4, 13.8, 11.5, 11.3. MS (ESI): (m/z) 373.0 [M⁺−Cl]⁺ (100).

### Example 6

### Synthesis of oxalic salt of (10S,13S)-3-(2-(4-(aminomethyl)-1H-1,2,3-triazol-1-yl)ethoxyimino)-10,13-dimethyltetradecahydro-1H-cyclopenta[a]phenanthren-17(2H)-one (Compound 31)

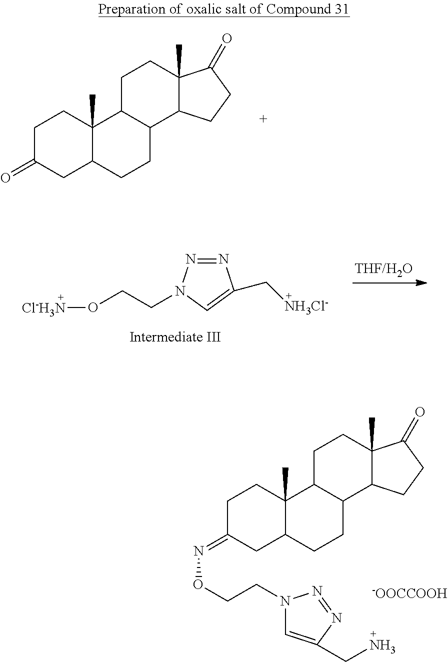

To a stirred solution of 5α-androstane-3,17-dione (50 mg, 0.173 mmol) in THF (1.2 mL), a solution of Intermediate III (40 mg, 0.173 mmol) in H₂O (0.5 mL) was added dropwise. After 2 h the reaction was complete and NaCl (66 mg) is added, the reaction mixture is stirred for an additional 15 min. The aqueous layer is extracted with THF (×2) and the combined organic phases are dried over Na₂SO₄, filtrated and evaporated under reduced pressure. The residue is purified by column chromatography eluted with CHCl₃/MeOH/NH₃ 95/5/1490/10/1 to afford 59 mg (80%) of the pure product. The product was dissolved in MeOH (0.3 mL), a stoichiometric amount of oxalic acid was added and the oxalic salt of Compound 31 was precipitated by the addition of Et₂O (5 mL), filtered and dried to afford 64 mg as a white solid. ¹H-NMR (300 MHz, CD₃OD) δ 8.02 (1H, s), 4.67 (2H, t, J=5.2 Hz), 4.33 (2H, t, J=5.2 Hz), 4.23 (2H, s), 3.02-2.98 (m, 2H) 2.81-2.77 (m, 1H), 2.48-0.73 (21H, m), 0.96 (3H, s), 0.88 (3H, s). ¹³C-NMR (CD₃OD) 222.4, 163.1, 161.5, 139.8, 124.4, 70.7, 54.0, 53.9, 51.2, 45.4, 38.2, 37.0, 36.0, 35.9, 35.2, 34.8, 34.0, 33.7, 31.3, 30.4, 28.3, 28.1, 27.6, 27.1, 21.3, 20.8, 20.1, 12.7, 10.3, 10.2.

### Example 7

### Synthesis of oxalic salt of (10S,13S)-3-(2-(4-(aminomethyl)-1H-1,2,3-triazol-1-yl)butoxyimino)-10,13-dimethyltetradecahydro-1H-cyclopenta[a]phenanthren-17(2H)-one (Compound 196)

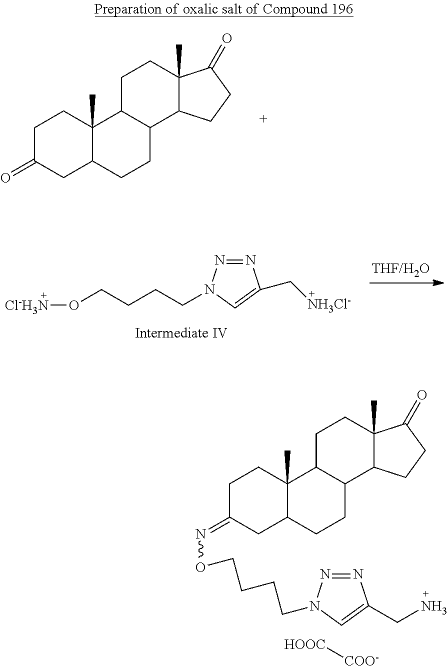

To a stirred solution of 5α-androstane-3,17-dione (100 mg, 0.347 mmol) in THF (2.3 mL), a solution of Intermediate IV (90 mg, 0.347 mmol) in H₂O (1.0 mL) was added dropwise. After 2 h the reaction was complete and NaCl (132 mg, 2.27 mmol) was added, and the reaction mixture was stirred for an additional 15 min and the two layers were separated. The aqueous layer was extracted with THF (×2) and the combined organic phases were dried over Na₂SO₄, filtered and evaporated under reduced pressure. The residue was purified by column chromatography elution solvent CHCl₃/MeOH/NH₃ 95/5/1→90/10/1 to afford 153 mg (97%) of Compound 196. Compound 196 was dissolved in MeOH (0.3 mL), a stoichiometric amount of oxalic acid was added and the oxalic salt of Compound 196 was precipitated by the addition of Et₂O (5 mL), filtered and dried to afford 149 mg as a white solid. ¹H-NMR (300 MHz, CD₃OD) δ 8.05 (1H, s), 4.46 (2H, t, J=6.8 Hz), 4.24 (2H, s), 3.98 (2H, t, J=6.0 Hz), 3.16-2.89 (m, 1H), 2.48-0.73 (25H, m), 0.97 (3H, s), 0.88 (3H, s); ¹³C-NMR (75 MHz, CD₃OD) δ 222.4, 163.1, 161.5, 139.7, 124.4, 70.7, 54.0, 51.1, 49.4, 45.3, 38.2, 37.0, 36.0, 35.2, 34.7, 34.0, 33.7, 31.3, 30.3, 28.2, 27.5, 27.1, 21.2, 20.8, 20.1, 12.7, 10.3.

### II. Biological Examples

### A. Material and Source Thereof

### Reagents

Ouabain, digoxin, paclitaxel, Testosterone-HSA (testosterone 3-(O-carboxymethyl)-oxime: human serum albumin) conjugates and fluorescein isothiocyanate (FITC) were purchased from Sigma (St. Louis, Mo.). Rostafuroxin, a well known Na+K+ ATPase inhibitor (Ferrari P et al, J Pharmacol Exp Ther. 1998 April; 285(1):83-94), was synthesized as reported in European Patent No. EP0583578.

### Synthesis of Fluorescent Testosterone-HSA-FITC (TAC) and HSA-FITC Conjugates

To label testosterone-HSA or HSA conjugates with FITC, a freshly prepared solution containing 6 mg of fluorescein isothiocyanate in 3 ml of 0.1M sodium carbonate buffer at pH 9.5 and 0° C. was added with stirring to 60 mg of a testosterone-human serum albumin conjugate (HSA) or HSA dissolved in 12 ml buffer and stirring was continued overnight at 4° C. The reaction mixture was placed in a dialysis tubing and dialyzed for five days against 2 liters of 10 mM ammonium bicarbonate NH₄HCO₃ at 4° C. Fresh dialysis solution was provided at 24 hour intervals. After dialysis, the sample was examined by thin layer chromatography to preclude the existence of free un-reacted testosterone or FITC and was subsequently lyophilized to dryness yielding 40.2 mg of Testosterone-HSA-FITC and 39.2 mg of HSA-FITC conjugate respectively. To determine the relative conjugation ratios of HSA to FITC in each case, 0.3 mg of Testosterone-HSA-FITC, HSA-FITC conjugate or FITC were dissolved in 2 ml of 0.05 M Tris buffer pH=8.4 and the absorbance at 252, 280 and 495 nm was measured. Based on the spectrophotometer results, testosterone-HSA-FITC conjugates contained 8.7 moles FITC per mole HSA, whereas HSA-FITC conjugates contained 7.62 moles FITC per mole HSA.

### Cell Lines

All cell lines were obtained from the American Type Culture Collection (Manassas, Va.) or the National Cancer Institute, NIH (Bethesda, Md., USA) and were adapted to grow in the commercially available culture media RPMI 1640, which was supplemented with 25 mM HEPES, 2 mM L-Glutamine, 5-10% fetal bovine serum and antibiotics in a 5% CO₂ humidified atmosphere (100%) at 37° C.

### NCI-ADR-RES Cells

NCI-ADR-RES cells have been classified as ovarian cancer cells by the National Cancer Institute.

### B. Assays and Results Thereof

Examples of the biological experiments as performed are described in this section, the examples, and the relevant literature, which in no way should be construed as being further limiting.

### Example 8

### Na⁺/K⁺-ATPase Inhibitory Activity of the Different Compounds

The inhibitory effect of a compound of the invention on ATPase activity was assessed in vitro using the colorimetric Adenosine 5′-Triphosphatase Enzymatic Assay of Sigma (St. Louis, Mo.) according to the manufacturer's instructions. This assay utilizes Adenosine 5′-Triphosphatase isolated from porcine cerebral cortex. Each reaction was performed in a final volume of 250 μl using 0.5 units/ml enzyme in the presence of different inhibitors. For the determination of the IC50 values of each compound, each inhibitor was added in five concentrations ranging from 10⁻⁵ to 10⁻⁹M (in triplicates). IC50 values were determined using the Origin® program software (OriginLab, Northampton, Mass.).

The Na⁺/K⁺-ATPase inhibitory activities of compound Nos. 120, 12, 61, 1, and 56 are summarized in Table 3. Ouabain and digoxin, two well known cardiotonic Na⁺/K⁺-ATPase inhibitors, were used as positive controls.

TABLE 3
Na+/K+-ATPase inhibitory activity of tested compounds

| Compound | Na+ K+ Inhibitory Activity (IC50, μM) |
|---|---|
| Comp 120 | 0.897 |
| Comp 12 | 0.375 |
| Comp 61 | 0.496 |
| Comp 1 | 0.124 |
| Comp 56 | 0.101 |
| Digoxin | 0.204 |
| Ouabain | 0.096 |

As shown in Table 3, all compounds being tested inhibited the activity of the Na+ K+ ATPase. The inhibitory activity of Ouabain was 96 nM, whereas digoxin had an IC50 value of 204 nM. Compound Nos. 120, 12 and 61 showed strong Na+K+ ATPase inhibitory activities with compound 12 having the strongest inhibitory activity (IC50=375 nM). Compound Nos. 1 and 56 were very potent Na+K+ ATPase inhibitors and inhibited enzyme activity with an IC50 value of 124 and 101 nM respectively. This activity was comparable to that of Ouabain and stronger than the one of Digoxin.

### Example 9

### Compounds of the Invention Inhibited the Proliferation and Survival of Neoplastic Cells

Cell proliferation/viability was assessed by the MTT [3-(4,5-dimethylthiazol-2-yl)-2,5-diphenyltetrazolium bromide] assay, which is commercially available (Sigma, St. Louis, Mo.). Cells were cultured in 96-well plates for twenty-four hours (7-10.000 cells/well) and incubated with various concentrations of the compounds of the invention in serum-containing medium (or left untreated) for seventy-two hours. At the end of incubation, the medium was aspirated and MTT dissolved in RPMI 1640 w/o phenol red was added to each well to a final concentration of 0.25 mg/ml. After four hours incubation in the dark (37° C., 5% CO₂) the supernatant was discarded. The converted dye (blue formazan crystals) was solubilized by adding 200 μl dimethylsulfoxide to each well. Absorbance was measured at 550 nm with reference at 655 nm using a spectrophotometer with the respective filters. All assays were performed in triplicate.

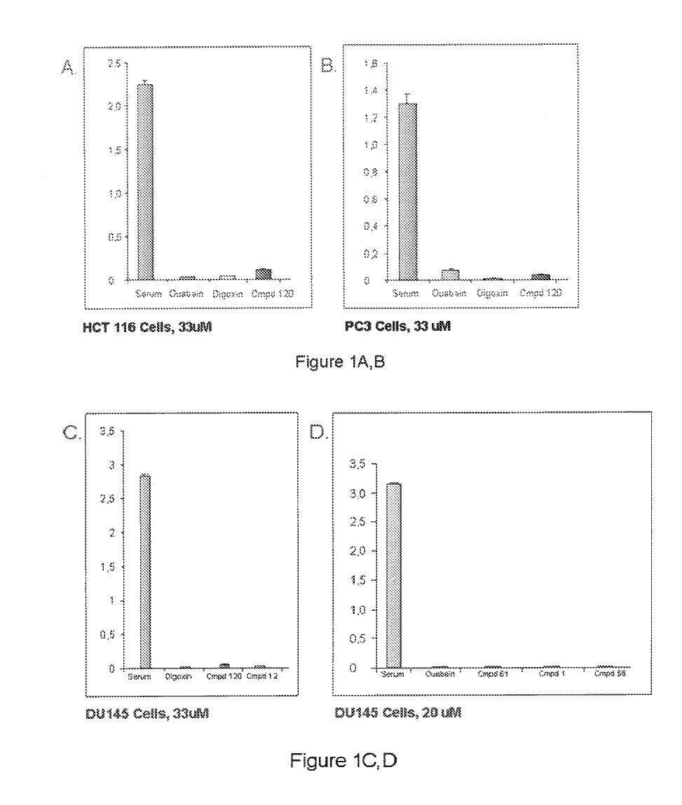

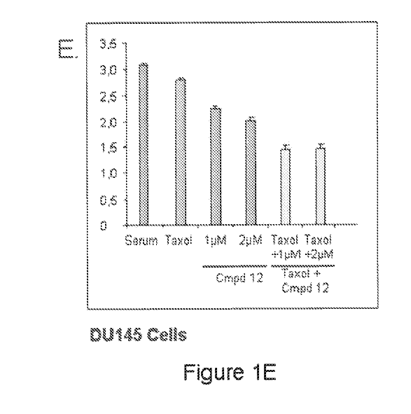

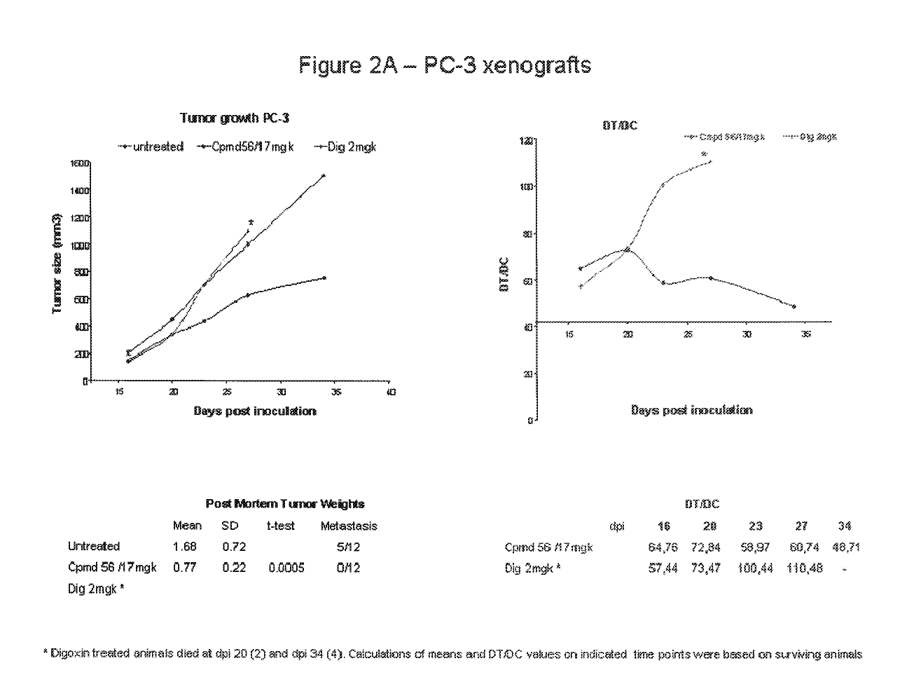

The effect of the different compounds on the proliferation and viability of cancer cell lines was tested. Compound 120 was tested in one colon cancer cell line (HCT116), and compound Nos. 120, 12, 61, 1, and 56 were tested in two prostate cancer cell lines (PC3 and DU145). Each of the cultures was exposed to various compound concentrations, and certain results were analyzed using a 3-(4,5-Dimethylthiazol-2-yl)-2,5-diphenyltetrazolium bromide (MTT) proliferation/viability assay. This colorimetric assay uses the reduction of MTT to formazan as a measure of metabolic activity. Untreated cells cultured in the presence of serum were used as a negative control. The effects of ouabain and digoxin, two well known cardiotonic steroid inhibitors of the Na⁺K⁺-ATPase, were assayed as positive controls. Absorbance results are shown in FIGS. 1A-1D.

As can be seen from FIGS. 1A-1D, the compounds of the invention (i.e., compound Nos. 120, 12, 61, 1, and 56) showed strong inhibition of cell proliferation and viability at the concentrations tested.

### Example 10

### Compound 12 Enhances the Cytotoxic Action of Paclitaxel in DU145 Prostate Cancer Cells

To evaluate whether compounds of the invention are capable of enhancing the cytotoxic action of paclitaxel (or “taxol”), MTT assays as described in Example 9 were performed using DU145 prostate cells treated with a concentration of paclitaxel, 1 or 2 uM of compound 12, and their combinations. These results are presented in FIG. 1E. Paclitaxel shows limited cytotoxic activity at the concentration tested (20 nM). FIG. 1E clearly shows that addition of compound 12 to paclitaxel clearly enhances the cytotoxic action of the latter compound in prostate cancer cells.

### Example 11

### Compound Nos 120, 12, 61, 1, and 56 had Potent Anti-Cancer Activity in 22 Different Cancer Cell Lines

SRB assays were performed according to standard National Cancer Institute Guidelines. Briefly, human tumor cells from different cancer panels were seeded into 96 well plates in 100 μL (plating densities ranging from 5-40,000 cells/well depending on the doubling time of individual cell lines) in serum-containing media for twenty-four hours prior to the addition of the compounds to be assayed. After 24 hours, one plate of each cell line was fixed in situ with trichloroacetic acid (TCA). The fixed slides were used to determine the cell population for each cell line at the time of drug addition (Tz). Each compound to be tested was solubilized in DMSO and the desired concentration was then added to the medium and diluted serially 1:10 to provide a total of five or ten drug concentrations plus control (in a final volume of 200 μL). The starting dose before any dilution was 100 uM for all compounds.

Following drug addition, each culture was incubated for forty-eight hours. The assay was terminated with the addition of cold TCA. The supernatant was discarded, and the plates were washed with tap water and air dried. Sulforhodamine B (SRB) solution was then added to each well. After staining, the bound stain was solubilized and the absorbance was read on an automated plate reader at a wavelength of 515 nm.

Using the absorbance measurements [time zero, (Tz), control growth, (C), and test growth in the presence of drug at the ten concentration levels (Ti)], the percentage growth was calculated at each of the drug concentrations levels. Percentage growth inhibition was calculated as:

[(Ti−Tz)/(C−Tz)]×100 for concentrations for which Ti>/=Tz

[(Ti−Tz)/Tz]×100 for concentrations for which Ti<Tz.

Three dose response parameters were calculated for each experimental agent. Growth inhibition of 50% (GI50) was calculated from [(Ti−Tz)/(C−Tz)]×100=50, which was the drug concentration resulting in a 50% reduction in the net protein increase (as measured by SRB staining) in control cells during the drug. The drug concentration resulting in total growth inhibition (TGI) was calculated from Ti=Tz. The LC50 (concentration of drug resulting in a 50% reduction in the measured protein at the end of the drug treatment as compared to that at the beginning) indicating a net loss of cells following treatment was calculated from [(Ti−Tz)/Tz]×100=−50.

Values were calculated for each of these three parameters when the level of activity was reached; however, if the effect was not reached or was exceeded, the value for that parameter was expressed as greater or less than the maximum or minimum concentration tested.

TABLE 4
In vitro characterization of Na⁺K⁺ ATPase inhibitors in tumor cell line panels

| Cell lines | Origin | Parameters | Paclitaxel | Ouabain | Digoxin | Cmpd 120 | Cmpd 12 | Cpmd 61 | Cmpd 1 | Cmpd 56 |
|---|---|---|---|---|---|---|---|---|---|---|
| A549 | Lung | GI50 | <0.001 | nd | <0.001 | 6.91 | 4.08 | 0.2070 | 0.3146 | <0.001 |
| | | TGI | 6.1384 | nd | 0.06 | 62.82 | 6.90 | 0.5149 | 0.5825 | 0.0040 |
| | | LC50 | 15.2586 | nd | 1.14 | 179.32 | 9.73 | 0.8228 | 0.8504 | 0.0082 |
| ERVK | Lung | GI50 | nd | nd | <0.01 | 4.57 | 1.45 | 0.4934 | 0.3384 | 0.0179 |
| | | TGI | nd | nd | 0.09 | 38.23 | 5.13 | 2.4710 | 0.6406 | 0.0628 |
| | | LC50 | nd | nd | 0.68 | 104.85 | 8.80 | 6.8378 | 0.9428 | 0.2884 |
| H160 | Lung | GI50 | <0.1 | <0.08 | <0.08 | 4.13 | 2.99 | nd | nd | nd |
| | | TGI | 3.7685 | 0.1 | <0.08 | 11.68 | 5.86 | nd | nd | nd |
| | | LC50 | >10 | 0.3 | 0.3 | 21.71 | 8.73 | nd | nd | nd |
| SKMEL28 | Melanoma | GI50 | 2.2985 | nd | 0.08 | 19.93 | 8.39 | 0.9927 | 0.5170 | 0.0409 |
| | | TGI | >10 | nd | 6.50 | 49.74 | 40.16 | 6.6230 | 0.9284 | 0.2253 |
| | | LC50 | >10 | nd | >100 | 79.56 | 85.64 | 12.3033 | 7.5369 | 3.7438 |
| LOXIMVI | Melanoma | GI50 | <0.001 | nd | 0.08 | 7.14 | 0.66 | nd | nd | nd |
| | | TGI | 0.288 | nd | >100 | 25.19 | 24.06 | nd | nd | nd |
| | | LC50 | 5.183 | nd | >100 | 97.23 | 116.88 | nd | nd | nd |
| UAC032 | Melanoma | GI50 | nd | nd | nd | nd | 4.57 | nd | nd | nd |
| | | TGI | nd | nd | nd | nd | 7.64 | nd | nd | nd |
| | | LC50 | nd | nd | nd | nd | 49.51 | nd | nd | nd |
| MB435 | Melanoma | GI50 | nd | nd | 0.07 | 7.29 | 5.45 | 0.6493 | 0.5985 | 0.0485 |
| | | TGI | nd | nd | 0.53 | 38.95 | 10.24 | 3.3784 | 1.3995 | 0.2182 |
| | | LC50 | nd | nd | 7.69 | 98.90 | 75.84 | 8.1980 | 6.3765 | 0.6679 |
| OVCAR5 | Ovary | GI50 | nd | nd | <0.01 | 20.47 | 5.30 | 3.8489 | 0.8337 | 0.0749 |
| | | TGI | nd | nd | 0.43 | 53.80 | 9.02 | 6.4549 | 3.7630 | 0.3411 |
| | | LC50 | nd | nd | 0.84 | 87.13 | 63.63 | 9.0608 | 7.3250 | 0.7405 |
| OVCAR3 | Ovary | GI50 | nd | nd | <0.01 | 6.50 | 3.01 | 0.3546 | 0.2907 | 0.0029 |
| | | TGI | nd | nd | 2.65 | 41.51 | 28.79 | 0.8004 | 0.6375 | 0.0114 |
| | | LC50 | nd | nd | 22.23 | >100 | 88.75 | 6.1226 | 0.9843 | 0.1699 |
| IGROV1 | Ovary | GI50 | 0.001 | nd | 0.04 | 5.49 | 5.37 | nd | nd | nd |
| | | TGI | 3.305 | nd | 0.35 | 10.74 | 12.38 | nd | nd | nd |
| | | LC50 | 10.565 | nd | >100 | 67.38 | 68.35 | nd | nd | nd |
| ADRRES | Ovary | GI50 | 1.0453 | nd | 0.04 | 8.73 | 3.08 | 0.5638 | 0.3210 | 0.0093 |
| | | TGI | 5.8034 | nd | 0.10 | 42.91 | 6.58 | 1.0156 | 0.6479 | 0.0491 |
| | | LC50 | 10.5615 | nd | 0.75 | 81.92 | 10.09 | 6.0657 | 0.9436 | 0.0908 |
| DU145 | Prostate | GI50 | <0.001 | nd | 0.03 | 5.87 | 3.04 | 0.3574 | 0.3324 | 0.0050 |
| | | TGI | 0.0086 | nd | 0.07 | 13.54 | 5.75 | 0.6262 | 0.5882 | 0.0220 |
| | | LC50 | 0.1434 | nd | 0.36 | 68.50 | 8.45 | 0.8951 | 0.8439 | 0.0706 |
| PCJ | Prostate | GI50 | <0.1 | <0.08 | <0.08 | 4.57 | 3.54 | 0.2807 | 0.2529 | 0.0332 |
| | | TGI | >10 | 0.1 | 0.1 | 14.22 | 6.03 | 1.8070 | 0.5751 | 0.3870 |
| | | LC50 | >10 | 0.3 | 0.3 | 29.37 | 8.52 | 7.4197 | 0.8973 | 0.9361 |
| SF295 | CNS | GI50 | nd | nd | nd | nd | 3.26 | nd | nd | nd |
| | | TGI | nd | nd | nd | nd | 6.15 | nd | nd | nd |
| | | LC50 | nd | nd | nd | nd | 9.04 | nd | nd | nd |
| U251 | CNS | GI50 | <0.001 | nd | 0.02 | 6.77 | 3.09 | nd | nd | nd |
| | | TGI | 1.745 | nd | 0.10 | 27.92 | 5.85 | nd | nd | nd |
| | | LC50 | 6.725 | nd | 0.61 | 68.49 | 8.61 | nd | nd | nd |
| SF268 | CNS | GI50 | <0.1 | <0.08 | <0.08 | 4.65 | 2.84 | 0.5667 | 0.3853 | 0.0567 |
| | | TGI | 1.9287 | 0.1 | <0.08 | 9.08 | 5.67 | 3.0203 | 0.6467 | 0.3490 |
| | | LC50 | >10 | 0.3 | 0.1 | 20.38 | 8.51 | 8.0309 | 0.9082 | 0.8105 |
| MCF7 | Breast | GI50 | <0.1 | <0.08 | <0.08 | 7.04 | 3.79 | nd | nd | nd |
| | | TGI | >10 | 0.2 | 0.1 | 14.85 | 6.63 | nd | nd | nd |
| | | LC50 | >10 | 0.3 | 0.4 | 24.06 | 9.47 | nd | nd | nd |
| T470 | Breast | GI50 | nd | nd | nd | nd | 5.98 | nd | nd | nd |
| | | TGI | nd | nd | nd | nd | 26.42 | nd | nd | nd |
| | | LC50 | nd | nd | nd | nd | 69.59 | nd | nd | nd |
| SU8686 | Pancreas | GI50 | 0.008 | nd | 0.03 | 7.45 | nd | nd | nd | nd |
| | | TGI | 7.927 | nd | 0.09 | 15.27 | nd | nd | nd | nd |
| | | LC50 | 16.517 | nd | 0.63 | 23.08 | nd | nd | nd | nd |
| HCT116 | Colon | GI50 | <0.1 | <0.08 | <0.08 | 4.28 | 4.06 | 0.2862 | 0.3641 | 0.0058 |
| | | TGI | 2.2397 | 4.5 | 0.7 | 16.06 | 7.38 | 1.3084 | 1.3056 | 0.0705 |
| | | LC50 | >10 | >50 | >50 | 39.21 | 10.71 | 9.2595 | 8.1050 | 0.7616 |
| HCT15 | Colon | GI50 | 0.053 | nd | 0.03 | 6.39 | 3.79 | nd | nd | nd |
| | | TGI | 0.852 | nd | 0.06 | 23.51 | 6.70 | nd | nd | nd |
| | | LC50 | 12.335 | nd | 0.16 | 76.31 | 9.61 | nd | nd | nd |
| CAKJ1 | Renal | GI50 | <0.001 | nd | <0.01 | 4.91 | 3.69 | 0.2685 | 0.3384 | <0.001 |
| | | TGI | 4.969 | nd | 0.07 | 9.90 | 6.56 | 1.2516 | 0.6430 | 0.0265 |
| | | LC50 | 11.586 | nd | 0.20 | 60.99 | 9.44 | 6.6512 | 0.9475 | 0.1718 |

Activity values: uM
nd: not done

Table 4 summarizes results characterizing the effects of compound Nos. 120, 12, 61, 1, and 56 on GI50, which measures the cell growth inhibitory power of the compound, TGI, which measures cytostatic effect (TGI) and LC50, which signifies cytotoxic effect. Each of these parameters in Table 4 was calculated in a sulforhodamine B (SRB) assay, which provides a colorimetric measure of a compound's anti-cancer activity. SRB assays were performed with compounds of the invention in 22 different cell lines (shaded in light gray in Table 4), which represent nine different tumor panels (lung, melanoma, ovary, prostate, CNS, breast, pancreas colon and renal). Ouabain and digoxin were used as controls. Paclitaxel was added as a non-Na⁺K⁺-ATPase inhibitor control.

Like the MTT assay, the results obtained with SRB showed that compound Nos. 120, 12, 61, 1, and 56 had potent anti-tumor activities (mostly single-digit micromolar GI50 action for compound Nos. 120 and 12, and nanomolar GI50 action for compound Nos. 61, 1 and 56) in all cell line panels tested. Compound 56 was the most potent compound having GI50 activity that was below 100 nanomolar for all cell line panels tested.

### Example 12

### Compound Nos. 120, 12, 61, 1, and 56 are Effective Against Multidrug Resistant Neoplasia

Table 4 also presents results with Compound Nos. 120, 12, 61, 1, and 56 in a well characterized drug resistant cell line, NCI-ADR-RES, that expresses high levels of MDR1 and P-glycoprotein. Consistent with the results in other cell lines of Table 4, Compound Nos. 120, 12, 61, 1, and 56 showed potent anti-tumor action in this multidrug resistant cell line. Most importantly, compound Nos. 120 and 12 exhibited the same GI50 value in multi-drug resistant NCI-ADR-RES cells to that observed in non-resistant MCF-7 cells. These results clearly indicate that compounds of the invention were just as effective in cells that were multidrug resistant as they were in other cancers. The results further suggest that compounds of the invention may provide an effective chemotherapeutic agent for the treatment of refractory and/or multidrug resistant tumors in subjects.

### Example 13

Compound 56 shows anti-tumor activity in human prostate and lung xenografts

Compound 56 was tested for its anti-tumor activity in PC-3 and A549 xenografts. To generate xenografts in mice, exponentially growing cultures of approximately 10⁶ PC-3 cells (prostate adenocarcinoma, grade IV) or A549 cells (lung carcinoma) were injected subcutaneously according to the British practice of bilateral trocar implants at the axillary region of 6-8 weeks old male (PC-3 cells) or female (A549 cells) Nod/Scid mice. The two tumor cell inoculums were added together for each mouse, thus producing a tumor burden per mouse value for data analysis. The advantage of the British system is reduced mouse-to-mouse variability and, thus, the ability to decrease the total number of animals per group, with five or six being sufficient for accurate data analysis of tumor response and evaluation of toxicities.

After having randomized the animals in groups, treatments started when the average tumor volume had reached about 100-200 mm³. Tumor volume was calculated according to the formula [(axb2)/2], where a=length and b=width of the tumor as measured with a vernier's caliper (measurements were performed twice a week). All administrations were intraperitoneal. Treated animals received a single injection according to the schedule described for each study (see below). In addition to tumor volume, the parameter, % DT/DC, was calculated, where DT=T−Do and DC=C−Do (Do is the average tumor volume at the beginning of the treatment; T and C are the volumes of treated and untreated tumors, respectively, at a specified day). Optimal DT/DC value was used as a measure of drug activity. Losses of weight, neurological disorders and behavioral and dietary changes were also recorded as indicators of toxicity (side effects). The experiment was terminated when tumor size in untreated animals reached a volume of about 1000-1500 mm³. All animals were treated according to Greek laws (2015/92), guidelines of the European Union and the European council (86/609 and ETS123, respectively), and Compliance with Standards for Human Care and Use of Laboratory Animals, NIH, USA (Assurance No. A5736-01).

### PC-3 Experiment

In order to develop PC-3 xenografts, male mice 6-8 weeks old were inoculated bilateral at the two axilla regions at day 0. Mice were observed for the development of tumors every two days by eye and palpation. At day 13 post inoculation (dpi), mean tumor volume was measured at 176.7 mm³. Subsequenbtly, mice were randomly divided into groups of 6 animals as described below: Group A: untreated animals; Group B: 17 mgk Compound 56 treated animals (Water For Injection; “WFI”); and Group C: 2 mgk (or mg/kg) digoxin treated animals (Phospate Buffer Saline; “PBS”).

Compound 56 was administered intraperitoneallly at the following dpis: d13, d14, d15, d16, d17, d20, d21, d22, d23, d24, d27, d28, 29, d30. Digoxin, was used as a positive control at a dose that has previously shown anti-cancer activity in a PC-3 xenograft model (Zhang H et al, 2008, PNAS, Vol. 105, No. 50, pp 19579-586). Digoxin's administration scheme was similar to the one of compound 56.

### A549 Experiment

In order to develop A549 xenografts, female mice 6-8 weeks old were inoculated bilateral at the two axilla regions at day 0. Mice were observed for the development of tumors every two days by eye and palpation. At day 20 post inoculation (dpi), mean tumor volume was measured at 117.6 mm³. Subsequenbtly, mice were randomly divided into groups of 6 animals as described below: Group A: untreated animals; Group B: paclitaxel 18 mgk (or mg/kg) (Cremophor/EtOH/WFI); and Group C: 20 mgk Compound 56 treated animals (WFI).

Compound 56 was administered intraperitoneallly using a [(QD×5;2) ×5] scheme. Paclitaxel, used as a positive control, was injected twice weekly for 5 cycles (Tuesday & Friday).

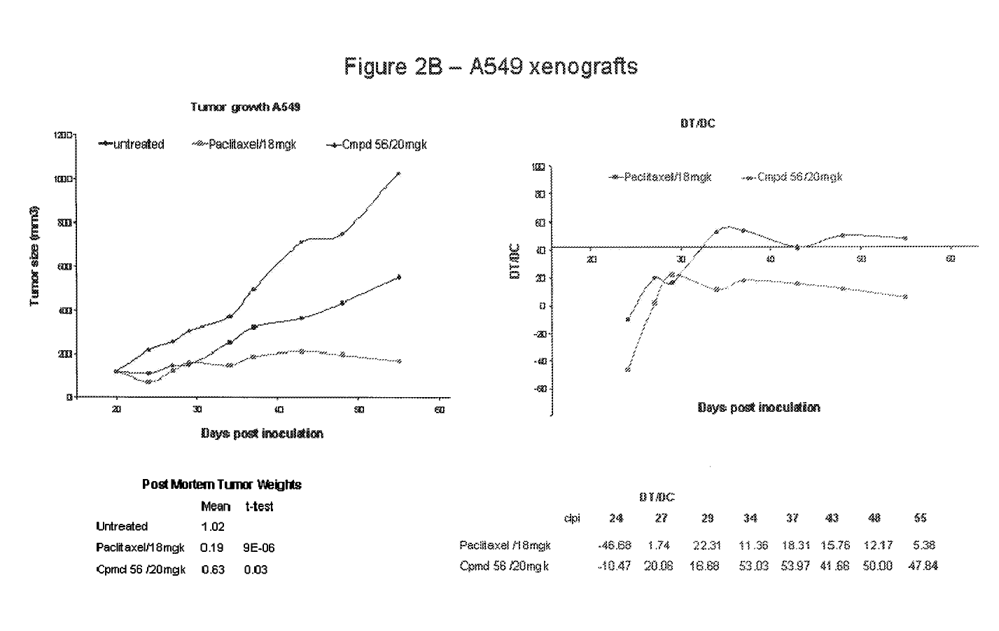

Results of the PC-3 and A549 experiments are presented in FIGS. 2A and 2B, and are further discussed herein.

FIG. 2A shows that compound 56 inhibited tumor growth in PC-3 xenografts with an optimal DT/DC of 48.71% at day 34 (p=6×10⁻⁶). Post mortem analysis on excised PC-3 tumors showed a statistical significant tumor growth inhibition of compound 56 and a suppression of bone lymph node and front axilla metastases in comparison to the control group. At the same time, digoxin was toxic as all animals died during the course of the experiment at dpi 20 (2 animals) and 34 (4 animals). Notably, compound 56 was not toxic under the experimental conditions used (as defined by body weight measurements).

In A549 xenografts, compound 56 had the optimal DT/DC at day 24 (−10.47%, p=0.0008), whereas paclitaxel had a DT/DC of −46,68% at day 24 (p=2,8×10⁻⁵). Post mortem analysis on excised tumors showed that there is a statistical significant tumor growth inhibition for both compound 56 and paclitaxel (FIG. 2B). Similarly to what was sen in PC-3 xenografts, no significant weight changes were observed with compound 56 in this model. These results prove that compound 56 has in vivo anti-tumor activity in PC-3 and A549 xenografts.

### Example 14

### Compound Nos. 12 and 56 Bind the Membrane Androgen Receptor

The membrane androgen receptor (mAR) is capable of transmitting rapid (non-genomic) androgen signals resulting in robust actin cytoskeleton re-organization in membrane androgen expressing cells (see Papadopoulou et al, IUBMB Life. 2009 January; 61(1):56-61). Membrane androgen receptors comprising testosterone-albumin conjugates are capable of inducing cell death—rather than proliferation—in AR-positive as well as AR-deficient cells, and testosterone-albumin conjugates have been shown to possess significant anti-cancer activity in various in vitro and in vivo models, independent of the status of classical androgen receptor (see, e.g., Papadopoulou et al, IUBMB Life. 2009 Jan., 61(1):56-61; PCT/IB03/02785; and Gatson et al, Endocrinology 2006, 147:2028-2034).

To assess whether compounds of the invention were capable of binding directly to the membrane androgen receptor, a fluorescent-based assay was used to measure the ability of a given compound to preclude binding of a fluorescent mAR ligand (testosterone-HSA-FITC conjugates) to its receptor in LNCaP and DU145 cells (see Kampa, M et al, 2002, Faseb J 16:1429-1431, and PCT/U52007/007913).

### Detection of Membrane Androgen Receptors

The detection of membrane androgen receptors using fluorescent testosterone-serum albumin conjugates was performed as described by Kampa, M et al, 2002, Faseb J 16:1429-1431, with minor modifications. Briefly, LNCaP or DU145 prostate cancer cells were cultured on 0.1% gelatin-coated glass coverslips in RPMI 1640 medium supplemented with 2 mM L-Glutamine, 10% Fetal Bovine Serum and 1% Penicillin/Streptomycin for at least 48 hours until reaching 70% confluency. After a 24 hour serum starvation period, cells were incubated with 40 μM testosterone-HSA-FITC for 1 hour at room temperature. The cells were then washed three times with phosphate buffered saline (PBS) and fixed with 3.7% formaldehyde in PBS for 5 minutes at room temperature. Human serum albumin-FITC was used as a control for background staining. After permeabilization with ice-cold acetone for 4 minutes at room temperature, cell nuclei were stained with DAPI. Coverslips were mounted on slides by using the Slow Fade/Antifade Reagent (Molecular Probes) and studied under a LEICA DMLB microscope, equipped with the appropriate fluorescence filters and a Leica DC 300F camera. Specimens were analyzed using the Leica FW4000 computer program.

### Membrane Androgen Receptor Competition Assays

To determine the capacity of a given compound to preclude binding of fluorescent testosterone-HSA conjugates (TAC) to the membrane androgen receptor, starved LNCaP or DU145 cells on coverslips prepared as described above were pre-treated with 40 μM of the indicated compound for 30 minutes prior to the addition of testosterone-HSA-FITC. At the end of the incubation period, cells were washed twice with PBS and 40 μM of testosterone-HSA-FITC was added. Specimens were prepared and analyzed as described above. Compounds binding to the membrane androgen receptor preclude binding of fluorescent testosterone-HSA conjugates to their target and abolish membrane-specific fluorescence.

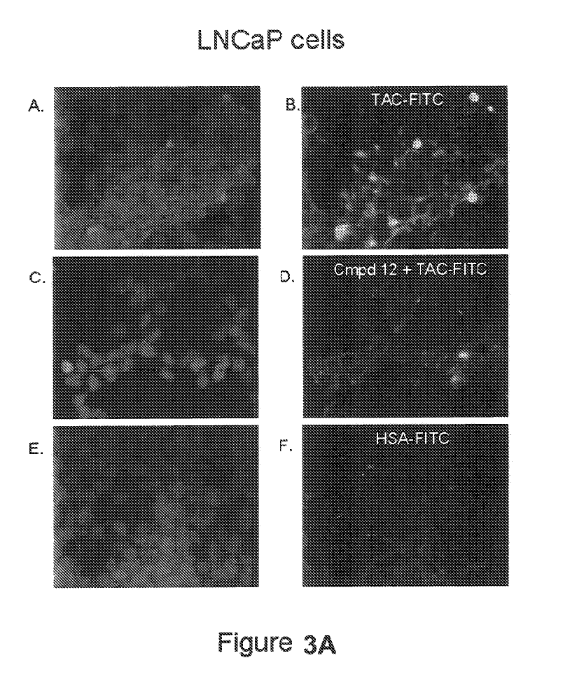

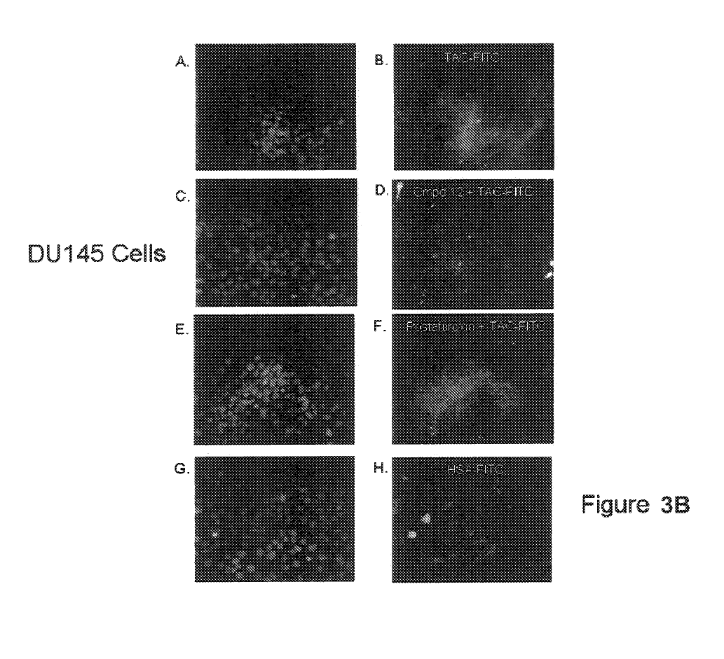

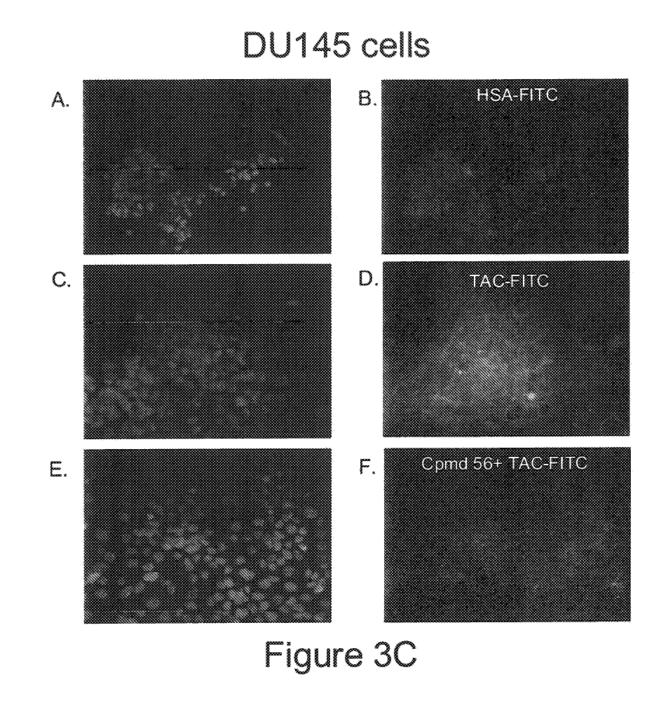

Results of the above assays are presented in FIGS. 3A-3C, and are further discussed herein.

FIGS. 3A and 3B are micrographs showing DAPI and membrane androgen receptor immunofluoresscent staining in LNCaP cells and DU145 cells treated with compound 12 relative to control cells. FIG. 3C provides micrographs showing DAPI and membrane androgen receptor immunofluorescent staining in DU145 cells obtained with TAC-FITC conjugates with or without treatment with compound 56.

As can be seen from the figures, testosterone-HSA-FITC (TAC-FITC) conjugates showed clear membrane staining as compared to control HSA-FITC conjugates (FIG. 3A: compare panels B and F; FIG. 3B: compare panels B and H, FIG. 3C: compare panels B and D). Cells pre-treated with testosterone 3-(O-carboxymethyl)oxime (Testo-CMO; the steroid moiety present in TAC-FITC conjugates) were devoid of membrane staining, indicating efficient competition for binding to the membrane androgen receptor. Interestingly, compound 12 and compound 56 pre-treated cells were also devoid of membrane fluorescence (FIG. 3A: compare panels B and D; FIG. 3B: compare panels B and D, FIG. 3C: compare panels D and F) indicating that compounds 12 and 56 preclude binding of TAC-FITC to the membrane androgen receptor. Similar results were obtained with compound 120. In contrast, another Na⁺K⁺ ATPase inhibitor used in this experiment, rostafuroxin, failed to block binding of TAC-FITC to mAR in DU145 cells (FIG. 3B: compare panel F to D, B). Notably all examined fields contained cells, as indicated by control DAPI stainings

Taken together these results indicate that compound Nos. 12 and 56 are capable of binding to the membrane androgen receptor. Furthermore, these results clearly suggest that specific Na⁺K⁺ ATPase inhibitors may function as membrane androgen receptor ligands. Since mAR is functionally implicated in cancer death by apoptosis, it is likely that the anti-cancer action of different Na⁺K⁺ ATPase inhibitors depends not only on their capacity to bind and inhibit Na+K+ ATPase, but also on their ability to bind to the membrane androgen receptor.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A compound of Formula (I) 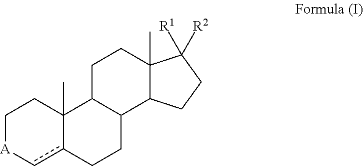
wherein: [symbol]
represents a single bond or double bond in the ring structure of Formula (I);
One of R¹ and R² is H, and the other is —OR³; alkyl optionally substituted with hydroxyl or alkoxy; —C(O)—NH₂; —C(O)—O-alkyl; —NHR⁸; alkynyl; or Wand R² together with the carbon to which they are attached form C═O, C═C(R⁹)₂, or CN—OR¹⁰;
R³ is H; —NO₂; carbonylalkyl; carbonylaryl; or aminoalkyl optionally substituted with one or more alkyl;
A is CH(OR⁴), C═N—OR⁵, CH—CH═N—OR⁶, C═CH—CH═N—OR⁶, CH—CH═CH—R⁶, CH—SR⁷, or CH—S(O)R⁷;
R⁴ is alkyl optionally substituted by a 4- to 6-membered heterocyclic ring, a 5 to 14-membered heteroaryl, amino(C₁₋C₄)alkoxyl, (C₁₋C₄)alkoxyl substituted by a 4- or 6-membered heterocyclic ring, or guanidinyl; aminoalkyl optionally substituted by one or more alkyl; or aminoacyl optionally substituted by alkyl; wherein said heterocyclic or heteroaryl ring is further optionally substituted by one or more alkyl
R⁵ is aminoalkyl optionally substituted by one or more alkyl or acetyl; alkyl optionally substituted by guanidinyl, heteroaryl that is further optionally substituted by one or more alkyl or aminoalkyl optionally substituted by one or more alkyl, or a 4- to 6-membered heterocyclic ring that is further optionally substituted by one or more alkyl; or a 4- to 6-membered heterocyclic ring optionally substituted by one or more alkyl;
R⁶ and R⁷, each independently, are aminoalkyl optionally substituted by one or more alkyl;
R⁸ is H or formyl;
R⁹, for each occurrence, is the same or different and is H or halogen;
R¹⁰ is H or alkyl; and
tautomers, stereoisomers, Z and E isomers, optical isomers, N-oxides, hydrates, polymorphs, pharmaceutically acceptable esters, salts, prodrugs and/or isotopic derivatives thereof.

2. The compound of claim 1, wherein R¹ and R² together with the carbon atom to which they are attached form C═O.

3. The compound of claim 2, wherein A is C═N—OR⁵.

4-9. (canceled)

10. The compound of claim 3, wherein R⁵ is (C₁-C₄)alkyl that is optionally substituted by guanidinyl, a 5- to 14-membered heteroaryl or a 4- to 6-membered heterocyclic ring, wherein 5- to 14-membered heteroaryl is optionally substituted by one or more (C₁-C₄)alkyl or amino(C₁-C₄)alkyl that is further optionally substituted by one or more (C₁-C₄)alkyl, and said 4- to 6-membered heterocyclic ring is optionally substituted by one or more (C₁-C₄)alkyl.

11-35. (canceled)

36. The compound of claim 10, wherein R⁵ is (C₁-C₄)alkyl that is substituted by a 4- to 6-membered heterocyclic ring, wherein said 4- to 6-membered heterocyclic ring is optionally substituted by one or more (C₁-C₄)alkyl.

37. The compound of claim 36, wherein R⁵ is (C₁-C₄)alkyl substituted by a pyrrolidinyl ring.

38. The compound of claim 37, wherein said compound of Formula (I) is:
13S)-10,13-dimethyl-3-(pyrrolidin-2-ylmethoxyimino)tetradecahydro-1H-cyclopenta[a]phenanthren-17(2H)-one (Compound 53): 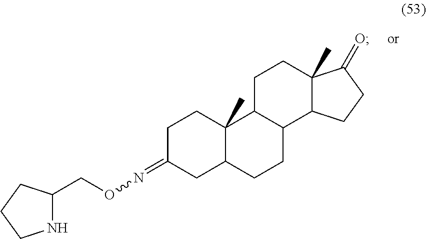
13S)-10,13-dimethyl-3-(pyrrolidin-2-ylmethoxyimino)-2,3,7,8,9,10,11,12,13,14,15,16-dodecahydro-1H-cyclopenta[a]phenanthren-17(6H)-one (Compound 54):

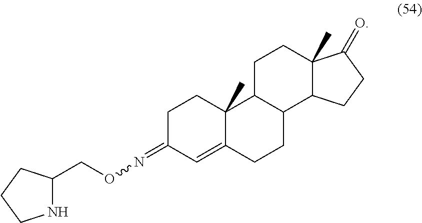

39. The compound of claim 10, wherein R⁵ is a 4- to 6-membered heterocyclic ring that is optionally substituted by one or more (C₁-C₄)alkyl.

40. The compound of claim 39, wherein R⁵ is a pyrrolidinyl, azetidinyl, or piperidinyl ring, which is optionally substituted by one or more (C₁-C₄)alkyl.

41. The compound of claim 40, wherein “ [symbol] ” represents a single bond in the ring structure of Formula (I).

42. The compound of claim 40, wherein said compound of Formula (I) is:
(10S,13S)-3-(azetidin-3-yloxyimino)-10,13-dimethyltetradecahydro-1H-cyclopenta[a]phenanthren-17(2H)-one (Compound 55): 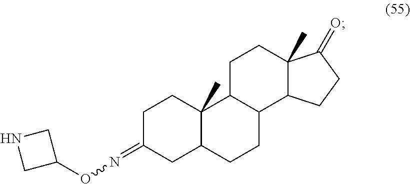
(10S,13S)-10,13-dimethyl-3-(pyrrolidin-3-yloxyimino)tetradecahydro-1H-cyclopenta[a]phenanthren-17(2H)-one (Compound 56): 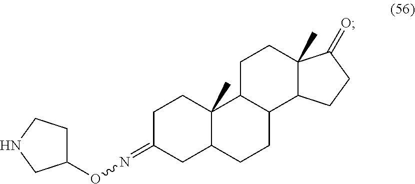
(10S,13S)-10,13-dimethyl-3-((S)-1-methylpyrrolidin-3-yloxyimino)tetradecahydro-1H-cyclopenta[a]phenanthren-17(2H)-one (Compound 57): 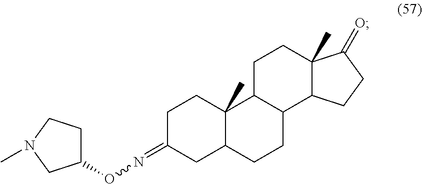
(10S,13S)-10,13-dimethyl-3-(piperidin-3-yloxyimino)tetradecahydro-1H-cyclopenta[a]phenanthren-17(2H)-one (Compound 58): 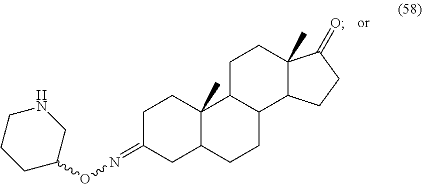
(10S,13S)-10,13-dimethyl-3-(piperidin-4-yloxyimino)tetradecahydro-1H-cyclopenta[a]phenanthren-17(2H)-one (Compound 59):

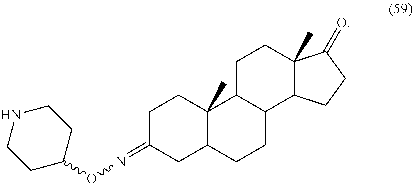

43. The compound of claim 40, wherein “ [symbol] ” represents a double bond in the ring structure of Formula (I).

44. The compound of claim 43, wherein said compound of Formula (I) is:
(10S,13S)-3-(azetidin-3-yloxyimino)-10,13-dimethyl-2,3,7,8,9,10,11,12,13,14,15,16-dodecahydro-1H-cyclopenta[a]phenanthren-17(6H)-one (Compound 60): 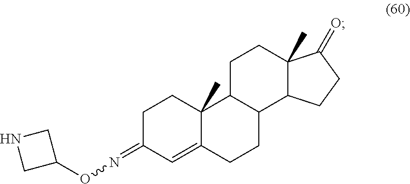
(10S,13S)-10,13-dimethyl-3-(pyrrolidin-3-yloxyimino)-2,3,7,8,9,10,11,12,13,14,15,16-dodecahydro-1H-cyclopenta[a]phenanthren-17(6H)-one (Compound 61): 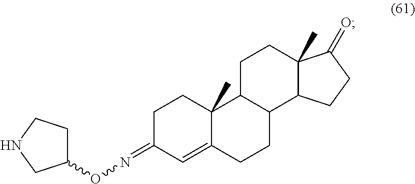
(10S,13S)-10,13-dimethyl-3-((S)-1-methylpyrrolidin-3-yloxyimino)-2,3,7,8,9,10,11,12,13,14,15,16-dodecahydro-1H-cyclopenta[a]phenanthren-17(6H)-one (Compound 62): 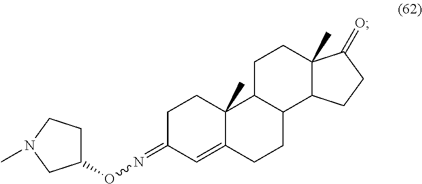
(10S,13S)-10,13-dimethyl-3-(piperidin-3-yloxyimino)-2,3,7,8,9,10,11,12,13,14,15,16-dodecahydro-1H-cyclopenta[a]phenanthren-17(6H)-one (Compound 63): 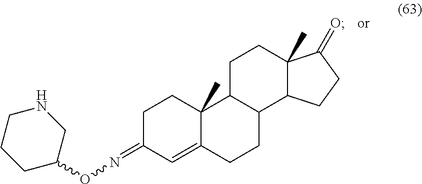
(10S,13S)-10,13-dimethyl-3-(piperidin-4-yloxyimino)-2,3,7,8,9,10,11,12,13,14,15,16-dodecahydro-1H-cyclopenta[a]phenanthren-17(6H)-one (Compound 64):

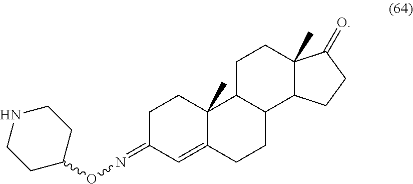

45-165. (canceled)

166. A method for reducing the growth, proliferation or survival of a neoplastic cell, the method comprising contacting the cell with an effective amount of a compound of claim 1, thereby reducing the growth, proliferation or survival of a neoplastic cell.

167-191. (canceled)

192. A composition comprising a therapeutically effective amount of a compound of claim 1 and a pharmaceutically acceptable excipient or carrier thereof.

193. The composition of claim 192, further comprising a therapeutically effective amount of a compound selected from the group consisting of vinca alkaloids, taxanes, epothilones, antifolates, purine analogs, pyrimidine analogs, DNA intercalators, topoisomerase inhibitors, topotecan, alkylating agents, platinum-based agents, receptor antagonists, hormone agents, anthracyclines, epipodophyllotoxins, antibiotics, antimicrotubule drugs, protein synthesis inhibitors, toxic peptides, enzyme inhibitors and anti-mitotics.

194-205. (canceled)

206. A method for treating or preventing prostate cancer in a subject, the method comprising administering to said subject an effective amount of a compound of claim 1, wherein said compound is capable of binding and inhibiting a Na+K+ ATPase, thereby preventing or treating said prostate cancer.

207-210. (canceled)

211. The method of claim 206, wherein the compound is selected from the group consisting of (1S,9aR,11aS)-17-hydroxy-10,13-dimethyl-6,7,8,9,10,11,12,13,14,15,16,17-dodecahydro-1H-cyclopenta[a]phenanthren-3(2H)-one O-2-aminoethyl oxime (Compound 120): 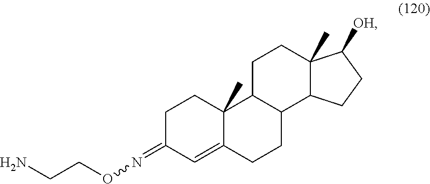
13S)-3-(2-aminoethoxyimino)-10,13-dimethyl-2,3,7,8,9,10,11,12,13,14,15,16-dodecahydro-1H-cyclopenta[a]phenanthren-17(6H)-one (Compound 12): 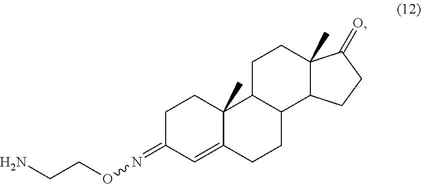
(10S,13S)-10,13-dimethyl-3-(pyrrolidin-3-yloxyimino)-2,3,7,8,9,10,11,12,13,14,15,16-dodecahydro-1H-cyclopenta[a]phenanthren-17(6H)-one (Compound 61): 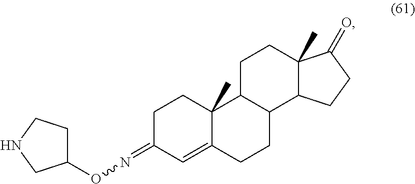
(10S,13S)-3-(2-aminoethoxyimino)-10,13-dimethyltetradecahydro-1H-cyclopenta[a]phenanthren-17(2H)-one (Compound 1): 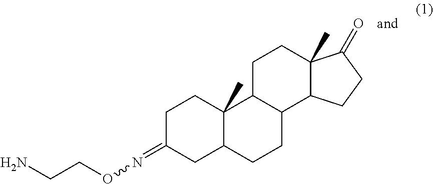
(10S,13S)-10,13-dimethyl-3-(pyrrolidin-3-yloxyimino)tetradecahydro-1H-cyclopenta[a]phenanthren-17(2H)-one (Compound 56):

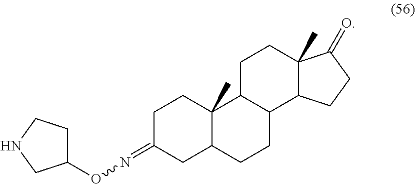

212-218. (canceled)

219. The compound of claim 3, wherein R⁵ is amino(C₁-C₄)alkyl optionally substituted by one or more alkyl; or R⁵ is (C₁-C₄)alkyl substituted by a heteroaryl that is substituted by an amino(C₁-C₄)alkyl

220. The compound of claim 219, wherein said compound of Formula (I) is selected from the group consisting of
(10S,13S)-3-(2-aminoethoxyimino)-10,13-dimethyltetradecahydro-1H-cyclopenta[a]phenanthren-17(2H)-one (Compound 1): 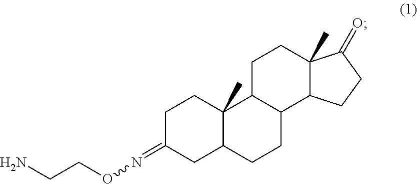
(10S,13S)-3-(2-(4-(aminomethyl)-1H-1,2,3-triazol-1-yl)ethoxyimino)-10,13-dimethyltetradecahydro-1H-cyclopenta[a]phenanthren-17(2H)-one (Compound 31): 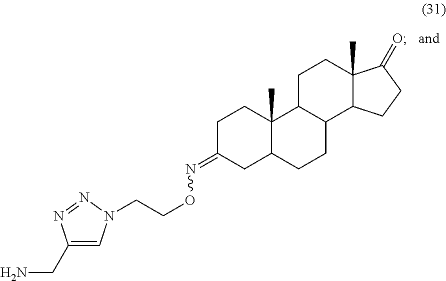
(10S,13S)-3-(2-(4-(aminomethyl)-1H-1,2,3-triazol-1-yl)butoxyimino)-10,13-dimethyltetradecahydro-1H-cyclopenta[a]phenanthren-17(2H)-one (Compound 196): [chemical structure]
